(19) United States
(12) Patent Application Publication
(10) Pub. No.: US 2026/0306628 A1
(43) Pub. Date: Oct. 8, 2026

(54) ALK7 COMBINATION TREATMENTS

(71) Applicant: Novo Nordisk A/S, Bagsvaerd (DK)

(72) Inventors: Johan Fredrik Paulsson, Malmoe (SE); Carlos Michel Castorena, Framingham, MA (US); Fiona McMurray, Frederiksberg (DK)

(21) Appl. No.: 19/630,561

(22) Filed: Mar. 27, 2026

Related U.S. Application Data
(60) Provisional application No. 63/780,661, filed on Mar. 31, 2025.

(30) Foreign Application Priority Data
May 28, 2025 (EP) 25179462.4

(51) Int. Cl. A61K 39/395; A61K 38/22; A61K 38/26; A61K 39/00; A61P 3/04

(52) U.S. Cl. CPC A61K 39/3955; A61K 38/22; A61K 38/26; A61P 3/04; A61K 2039/505

(57) ABSTRACT

The present disclosure provides an anti-ALK7 receptor antibody or antigen binding fragment thereof for use in prevention and/or treatment of overweight, obesity, and/or diabetes in combination with a second biological active agent, such as a GLP-1 receptor agonist, e.g., a GLP-1 receptor-amylin receptor agonist; anti-GIP receptor antibody or antigen binding fragment thereof, and/or an anti-ActRII antibody or antigen binding fragment thereof.

### TECHNICAL FIELD

The present disclosure provides combination therapies for the treatment of diseases or disorders, such as metabolic diseases or disorders, including but not limited to overweight, obesity and/or diabetes.

### CROSS-REFERENCE TO RELATED APPLICATIONS

This application claims priority under 35 U.S.C. § 119 to U.S. Provisional Application No. 63/780,661, filed Mar. 31, 2025, and claims priority to European Patent Application 25179462.4, filed May 28, 2025; all of which are hereby incorporated by reference.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on Mar. 25, 2026, is named 250013US02.xml and is 119,206 bytes in size.

### BACKGROUND

Overweight and obesity are abnormal or excessive accumulation of body fat that present a risk to an individual's overall health. A body mass index (BMI) over 25 is considered overweight, and a BMI of over 30 is considered obese. Obesity is a leading risk factor in a large number of serious conditions, including type 2 diabetes and its associated co-morbidities, and cardiovascular diseases such as heart disease and stroke, which are the leading causes of death worldwide. Obesity is now recognized by the World Health Organization (WHO) as an issue that has grown to epidemic proportion, even in children: in 2016, 1.9 billion adults worldwide were reportedly obese; in 2019, 38.3 million children under the age of 5 worldwide were reportedly obese. According to the WHO, 422 million people worldwide have diabetes and 1.6 million deaths are directly attributed to diabetes each year.

Activin receptor-like kinase 7 (ALK7, also known as ACVR1C) is a single transmembrane type 1 serine/threonine kinase within the TGF-β receptor family, primarily expressed on mature adipocytes in humans and relevant animal models e.g. rodents. The signalling complex is a heterooligomer that consists of a heterotetrameric receptor complex with two type I and two type II transmembrane serine/threonine kinases that is activated by homodimeric ligands of the TGF-β superfamily, including Activin B, Activin C, and Activin E. In the signalling complex, the type II receptors phosphorylate and activate the type I receptors, which phosphorylate and then bind and activate the SMAD transcriptional regulators, SMAD2 and SMAD3. Activation of ALK7 promotes lipid storage in adipose tissue. Inhibition of the ALK7, including inhibition with neutralizing antibodies, has been shown to reduce total body weight. ALK7 antibodies have previously been described and characterized in patent applications such as WO17/1805037, WO19/084249 and WO20/244343.

### SUMMARY

The present disclosure provides additional treatment options for the increasing number of patients that suffer from metabolic disorders and diseases and in particular for patients suffering from type 2 diabetes, obesity, overweight, or combinations thereof.

The disclosure provides combinations, such as combination therapies, comprising an anti-ALK7 antibody or antigen binding fragment thereof and a second biological active agent, as well as uses of such combination therapies for the prevention and/or treatment of overweight, obesity, and/or diabetes, e.g., type 2 diabetes. The present disclosure particularly provides combination therapies comprising an anti-ALK7 antibody or antigen binding fragment thereof and a second biological active agent selected from a GLP-1 receptor agonist, a GLP-1 receptor-amylin receptor agonist, an anti-GIP receptor antibody or antigen binding fragment thereof, an anti-ActRII antibody or antigen binding fragment thereof, or a combination thereof. As provided herein, the inventors have observed synergy for combinations of an anti-ALK7 antibody or antigen binding fragment thereof and a second biological active agent, such as a GLP-1 receptor agonist, a GLP-1 receptor-amylin receptor agonist, an anti-GIP receptor antibody or antigen binding fragment thereof, or an anti-ActRII antibody or antigen binding fragment thereof in mice.

By administering a combination of an anti-ALK7 antibody or antigen binding fragment thereof and a second biological active agent, such as a GLP-1 receptor agonist, a GLP-1 receptor-amylin receptor agonist, an anti-GIP receptors antibody or antigen binding fragment thereof, or an anti-ActRII antibody or antigen binding fragment thereof, increased total weight loss, due to fat mass loss while preserving lean mass was observed, comparable to administration of the anti-ALK7 antibody or antigen binding fragment thereof or second biological active agent alone.

Thus, the present disclosure provides combinations, such as combination therapies, and/or compositions comprising an anti-ALK7 antibody or antigen binding fragment thereof and a second biological active agent useful in methods of reducing weight or inducing weight loss in a subject, such as a human subject, as well as prevention and/or treatment of overweight, obesity, diabetes, metabolic diseases or disorders, and cardiovascular diseases (CVD). The present disclosure provides an anti-ALK7 antibody or antigen binding fragment thereof for use as a medicament in combination with a second biological active agent.

In some embodiments, the present disclosure provides an anti-ALK7 antibody or antigen binding fragment thereof for use in a method of prevention and/or treatment of overweight, obesity, and/or diabetes, wherein the anti-ALK7 antibody or antigen binding fragment thereof is administered to a subject in combination with a second biological active agent.

In some embodiments, the present disclosure provides a second biological active agent for use in a method of prevention and/or treatment of overweight, obesity, and/or diabetes, wherein the second biological active agent is administered to a subject in combination with an anti-ALK7 antibody or antigen binding fragment thereof.

Administration of a GLP-1 receptor agonist (e.g., semaglutide) alone resulted in reduced total body mass resulting from a reduction in lean body mass and reduction in body fat mass. Surprisingly, administration of an anti-ALK7 receptor antibody or antigen binding fragment thereof in combination with a GLP-1 receptor agonist (e.g., semaglutide) resulted in a improved total body weight loss compared to administration of an anti-ALK7 receptor antibody or antigen binding fragment thereof or a GLP-1 receptor agonist alone. The improved total body weight loss was predominantly due to a reduction in body fat mass while lean body mass preserved (see Example 5).

Administration of a GLP-1 receptor-amylin receptor agonist alone resulted in a reduction of total body weight resulting from a reduction in lean body mass and a reduction in fat mass. However, administration of an anti-ALK7 receptor antibody or antigen binding fragment thereof in combination with a GLP-1 receptor-amylin receptor agonist results in improved total body weight loss primarily driven by fat mass loss. The reduction in lean body mass observed when administering a GLP-1 receptor-amylin receptor agonist alone was blunted when an anti-ALK7 receptor antibody or antigen binding fragment thereof was co-administered (see Example 6).

Administration of an anti-GIP receptor antibody or antigen binding fragment thereof alone resulted in total body weight loss, due to a reduction in both fat mass and lean mass. Whereas, administering an anti-ALK7 receptor antibody or antigen binding fragment thereof in combination with an anti-GIP receptor antibody or antigen binding fragment thereof resulted in a greater total body weight loss; greater total fat mass loss, while preserving lean mass (see Example 7).

Administration of an anti-ActRII antibody or antigen binding fragment thereof alone resulted in an altered body composition, with a reduction in fat mass and an increase in lean mass. Administration of an anti-ALK7 receptor antibody or antigen binding fragment thereof in combination with an anti-ActRII antibody or antigen binding fragment thereof resulted in a total body weight loss; due to total fat mass loss, while preserving lean mass (see Example 8).

Combination therapies provided herein provide means for modulating body composition in a specific manner, such as targeting fat mass loss while preventing lean mass loss or even increasing lean body mass. Such combination therapies may be useful when preventing and/or treating particular diseases and/or disorders where a reduction in lean body mass may not be favorable when losing total body weight.

The present disclosure provides an anti-ALK7 receptor antibody or antigen binding fragment thereof for use in prevention and/or treatment of overweight, obesity, and/or diabetes in combination with a second biological active agent, wherein the second biological agent comprises a GLP-1 receptor agonist. In some embodiments, an anti-ALK7 receptor antibody or antigen binding fragment thereof is administered (i) together with the second biological active agent, or (ii) concomitantly or sequentially with the second biological active agent. In some embodiments, a GLP-1 receptor agonist comprises an amino acid sequence having at least 90% sequence identity to the amino acid sequence of SEQ ID NO: 2. In some embodiments, a GLP-1 receptor agonist comprises the following structure (Formula I (SEQ ID NO: 106)):

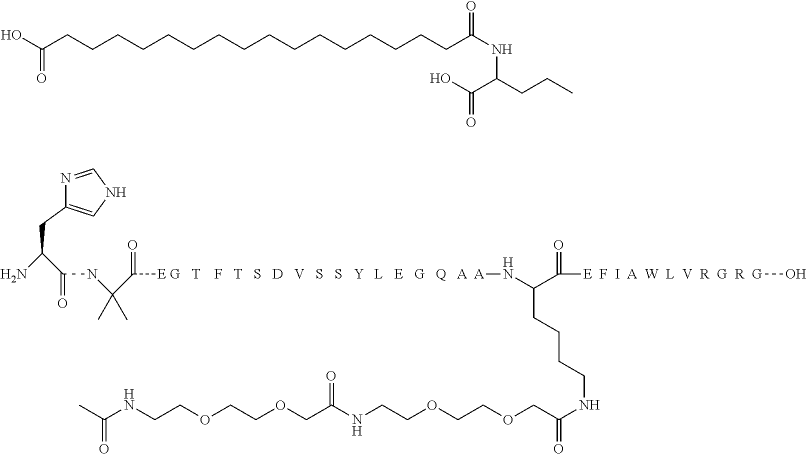

wherein the amino acid sequence is SEQ ID NO: 2, or a pharmaceutically acceptable salt thereof. In some embodiments, a GLP-1 receptor agonist comprises the following structure (Formula II (SEQ ID NO: 107)):

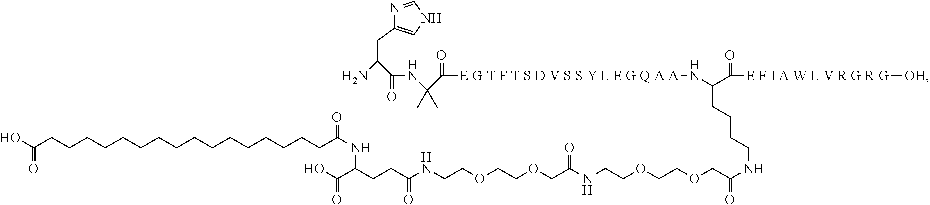

wherein the amino acid sequence is SEQ ID NO: 2, or a pharmaceutically acceptable salt thereof.

In some embodiments, a second biological active agent comprises a GLP-1 receptor agonist and an amylin receptor agonist. In some embodiments, a second biological active agent comprises a GLP-1 receptor-amylin receptor agonist. In some embodiments, a GLP-1 receptor-amylin receptor agonist comprises an amino acid sequence having at least 90% sequence identity to the amino acid sequence of SEQ ID NO: 3. In some embodiments, a GLP-1 receptor-amylin receptor agonist comprises the following structure (Formula IV (SEQ ID NO: 108)): 10

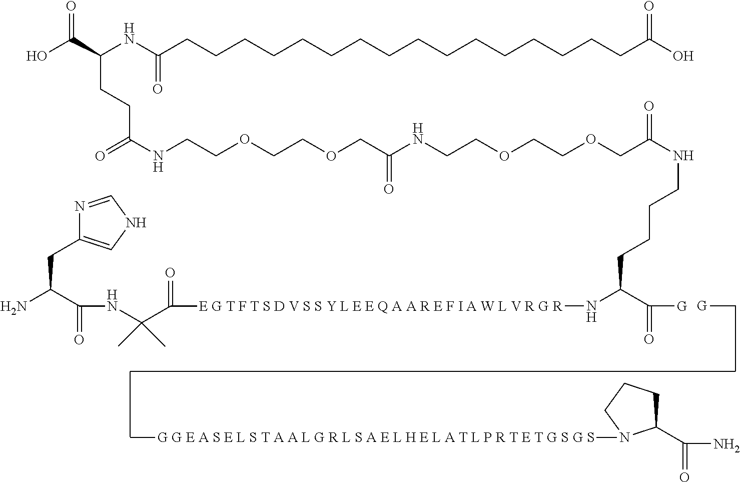

wherein the amino acid sequence is SEQ ID NO: 3, or a pharmaceutically acceptable salt thereof. In some embodiments, a GLP-1 receptor-amylin receptor agonist comprises the following structure (Formula V (SEQ ID NO: 109)):

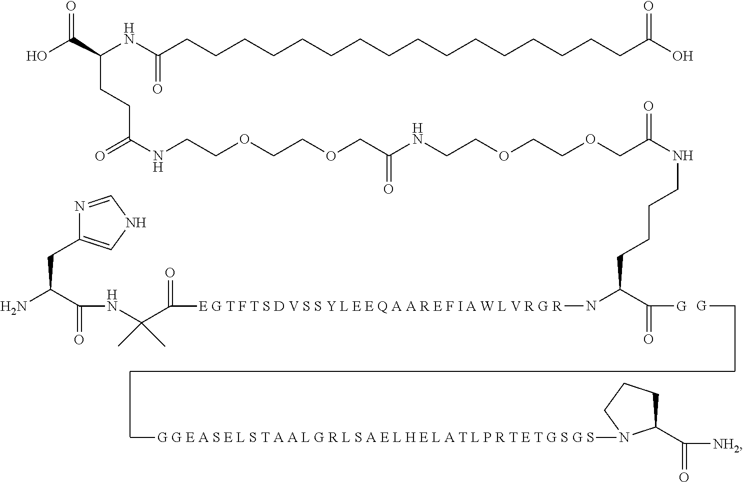

wherein the amino acid sequence is SEQ ID NO: 3, or a pharmaceutically acceptable salt thereof.

In some embodiments, an anti-ALK7 receptor antibody or antigen binding fragment thereof is (i) an antagonist; (ii) characterized in that it blocks binding of activin B to an ALK7 receptor; and/or (iii) characterized in that it blocks formation of a hexameric signaling complex of an ALK7 receptor with an ActRIIA/B Type II receptor. In some embodiments, an anti-ALK7 receptor antibody or antigen binding fragment thereof comprises three heavy chain CDR sequences, hCDR1-3, and three light chain CDR, sequences, ICDR1-3, wherein
a) hCDR1 is SGNYWN (SEQ ID NO: 8) or SGSYWN (SEQ ID NO: 9) with up to 1 amino acid substitutions,
b) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid substitutions,
c) hCDR3 is DYYGSEGFGY (SEQ ID NO: 17) or DYYGSEGFAY (SEQ ID NO: 18) with up to 1 or 2 amino acid substitutions,
d) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
e) ICDR2 is WTSTRES (SEQ ID NO: 22) or WASTRES (SEQ ID NO: 23) with up to 1 amino acid substitutions, and
f) ICDR3 is KQSYNLPYT (SEQ ID NO: 24) or KRSYNLPYT (SEQ ID NO: 25) with up to 1 amino acid substitutions.

In some embodiments, an anti-ALK7 receptor antibody or antigen binding fragment comprises three heavy chain CDR sequences, hCDR1-3, and three light chain CDR, sequences, ICDR1-3, wherein
i. the three heavy chain CDR sequences are selected from the CDR sequences of any one of the heavy chain variable regions (HV) identified by SEQ ID NO: 26-59, and/or
ii. the three light chain CDR sequences are selected from the CDR sequences of any one of the light chain variable regions (LV) identified by SEQ ID NO: 60-84.

In some embodiments, uses provided herein comprises body fat mass reduction. In some embodiments, uses provided herein comprises lean body mass preserving.

### BRIEF DESCRIPTION OF DRAWING

The Drawing included herein, which is composed of the following Figures, is for illustration purposes only and not for limitation.

FIGS. 1A-D: A) Relative change in body weight from baseline (% Delta); B) Daily food intake (g); C) Relative change in fat mass from baseline (% Delta); D) Relative change in fat mass from baseline (% Delta). Open circles represent controls; open triangles pointing up represent administration of an exemplary anti-ALK7 antibody; open triangles pointing down represent administration of an exemplary GLP-1 receptor agonist; open squares represent administration of a combination of an exemplary anti-ALK7 antibody and an exemplary GLP-1 receptor agonist.

FIGS. 2A-C: A) Fasting blood glucose (mg/dL); B) Fasting plasma insulin (ng/mL); C) Liver mass, and fat and lean mass composition (g). Open circles represent controls; open triangles pointing up represent administration of an exemplary anti-ALK7 antibody; open triangles pointing down represent administration of an exemplary GLP-1 receptor agonist; open squares represent administration of a combination of an exemplary anti-ALK7 antibody and an exemplary GLP-1 receptor agonist.

FIGS. 3A-D: A) Relative change in body weight from baseline (% Delta); B) Daily food intake (g); C) Relative change in fat mass from baseline (% Delta); D) Relative change in fat mass from baseline (% Delta). Open circles represent controls; open triangles pointing up represent administration of an exemplary anti-ALK7 antibody; open triangles pointing down represent administration of an exemplary GLP-1 receptor-amylin receptor agonist (1 nmol/kg); closed triangles pointing down represent administration of an exemplary GLP-1 receptor-amylin receptor agonist (3 nmol/kg); open squares represent administration of a combination of an exemplary anti-ALK7 antibody and an exemplary GLP-1 receptor-amylin receptor agonist (1 nmol/kg); closed squares represent administration of a combination of an exemplary anti-ALK7 antibody and an exemplary GLP-1 receptor-amylin receptor agonist (3 nmol/kg).

FIGS. 4A-C: A) Fasting blood glucose (mg/dL); B) Fasting plasma insulin (ng/ml); C) Liver mass, and fat and lean mass composition (g). Open circles represent controls; open triangles pointing up represent administration of an exemplary anti-ALK7 antibody; open triangles pointing down represent administration of an exemplary GLP-1 receptor-amylin receptor agonist (1 nmol/kg); closed triangles pointing down represent administration of an exemplary GLP-1 receptor-amylin receptor agonist (3 nmol/kg); open squares represent administration of a combination of an exemplary anti-ALK7 antibody and an exemplary GLP-1 receptor-amylin receptor agonist (1 nmol/kg); closed squares represent administration of a combination of an exemplary anti-ALK7 antibody and an exemplary GLP-1 receptor-amylin receptor agonist (3 nmol/kg).

FIGS. 5A-D: A) Relative change in body weight from baseline (% Delta); B) Daily food intake (g); C) Relative change in fat mass from baseline (% Delta); D) Relative change in fat mass from baseline (% Delta). Open triangles pointing up represent administration of an exemplary anti-ALK7 antibody; open triangles pointing down represent administration of an exemplary anti-GIP receptor antibody; open squares represent administration of a combination of an exemplary anti-ALK7 antibody and an exemplary anti-GIP receptor antibody.

FIGS. 6A-C: A) Fasting blood glucose (mg/dL); B) Fasting plasma insulin (ng/mL); C) Liver mass, and fat and lean mass composition (g). Open triangles pointing up represent administration of an exemplary anti-ALK7 antibody; open triangles pointing down represent administration of an exemplary anti-GIP receptor antibody; open squares represent administration of a combination of an exemplary anti-ALK7 antibody and an exemplary anti-GIP receptor antibody.

FIGS. 7A-D: A) Relative change in body weight from baseline (% Delta); B) Daily food intake (g); C) Relative change in fat mass from baseline (% Delta); D) Relative change in fat mass from baseline (% Delta). Open circles represent controls; open triangles pointing up represent administration of an exemplary anti-ALK7 antibody; open triangles pointing down represent administration of an exemplary anti-ActRII antibody; open squares represent administration of a combination of an exemplary anti-ALK7 antibody and an exemplary anti-ActRII antibody.

FIGS. 8A-C: A) Fasting blood glucose (mg/dL); B) Fasting plasma insulin (ng/ml); C) Liver mass, and fat and lean mass composition (g). Open circles represent controls; open triangles pointing up represent administration of an exemplary anti-ALK7 antibody; open triangles pointing down represent administration of an exemplary anti-ActRII antibody; open squares represent administration of a combination of an exemplary anti-ALK7 antibody and an exemplary anti-ActRII antibody.

FIGS. 9A-D: A) Relative change in body weight from baseline (% Delta); B) Daily food intake (g); C) Relative change in fat mass from baseline (% Delta); D) Relative change in fat mass from baseline (% Delta). Open circles represent controls; open triangles pointing up represent administration of an exemplary anti-ALK7 antibody; open triangles pointing down represent administration of an exemplary GLP-1 receptor agonist; open square represent administration of the an exemplary of a combination of GLP-1 receptor agonist and anti-ALK7i antibody; open diamonds represent administration of an exemplary GLP-1 receptor-amylin receptor agonist; closed squares represent administration of a combination of an exemplary anti-ALK7 antibody and an exemplary GLP-1 receptor-amylin receptor agonist.

FIGS. 10A-C: A) Fasting blood glucose (mg/dL); B) Fasting plasma insulin (ng/ml); C) Liver mass, and fat and lean mass composition (g). Open circles represent controls; open triangles pointing up represent administration of an exemplary anti-ALK7 antibody; open triangles pointing down represent administration of an exemplary GLP-1 receptor agonist; open square represent administration of the an exemplary of a combination of GLP-1 receptor agonist and anti-ALK7i antibody; open diamonds represent administration of an exemplary GLP-1 receptor-amylin receptor agonist; closed squares represent administration of a combination of an exemplary anti-ALK7 antibody and an exemplary GLP-1 receptor-amylin receptor agonist.

### CERTAIN DEFINITIONS

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological-setting). In some embodiments, affinity is assessed relative to a reference, e.g., a reference that has a known affinity above a particular threshold (a “positive control” reference) or a reference that has a known affinity below a particular threshold (a “negative control” reference”). In some embodiments, affinity may be assessed relative to a contemporaneous reference; in some embodiments, affinity may be assessed relative to a historical reference. Typically, when affinity is assessed relative to a reference, it is assessed under comparable conditions.

Agonist: The term “agonist” may be used herein to refer to a ligand, agent, condition, or event whose presence, level, degree, type, or form correlates with increased level or activity of another agent (i.e., the agonized agent or the target agent). In general, an agonist may be or include an agent of any chemical class including, for example, polypeptides, small molecules, nucleic acids, carbohydrates, lipids, metals, and/or any other entity that shows the relevant activating activity. In some embodiments, an agonist may be direct (in which case it exerts its influence directly upon its target). In some embodiments, an agonist may be indirect (in which case it exerts its influence by other than binding to its target, e.g., by interacting with a regulator of the target, so that level or activity of the target is altered).

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Non-standard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Amylin: The term “amylin” herein refers to a polypeptide having the same amino acid sequence as an endogenous amylin, such as human amylin.

Amylin receptor agonist: An “amylin receptor agonist” as used herein refers to a chemical entity which is characterized in that it binds to an amylin receptor and is capable of activating it. In some embodiments, an “amylin receptor agonist” is characterized in that it binds to and activates at least an amylin receptor 3 (AMYR3) complex. In some embodiments, an amylin receptor agonist is characterized in that it is capable of agonising the calcitonin receptor and AMYR1-2.

Antagonist: Those skilled in the art will appreciate that the term “antagonist”, as used herein, may be used to refer to an agent, condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies; antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′) 2, Fab′, di-sdFv, single domain antibodies, trifunctional antibodies, diabodies, and minibodies, etc. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Antigen binding fragment thereof: As used herein, an “antigen binding fragment thereof” refers to a portion of an antibody or antibody agent as described herein binds an antigen or portion thereof. An antigen binding fragment thereof may be produced by any means. For example, in some embodiments, an antigen binding fragment thereof may be enzymatically or chemically produced by fragmentation of an intact antibody or antibody agent. Alternatively, in some embodiments, an antigen binding fragment thereof may be recombinantly produced (i.e., by expression of an engineered nucleic acid sequence. In some embodiments, an antigen binding fragment thereof may be wholly or partially synthetically produced. In some embodiments, an antigen binding antibody fragment may have a length of at least about 50, 60, 70, 80, 90, 100, 110, 120, 130, 140, 150, 160, 170, 180, 190 amino acids or more, in some embodiments at least about 200 amino acids.

Combination therapy: As used herein, the term “combination therapy” refers to a clinical intervention in which a subject is exposed to, e.g., simultaneously exposed to, two or more therapeutic regimens (e.g. two or more therapeutic agents or modalities). “Combination therapy” is used herein interchangeably with “therapeutic combination”. In some embodiments, the two or more therapeutic regimens may be administered simultaneously. In some embodiments, the two or more therapeutic regimens may be administered sequentially (e.g., a first regimen administered prior to administration of any dose or episode of a second regimen). In some embodiments, two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points.

Complementarity Determining Region or CDR: The term “CDR” means a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

GLP-1: The term “GLP-1” or “native GLP-1” herein refers to human Glucagon-Like Peptide-1 (GLP-1(7-37)), shown in the sequence listing as SEQ ID NO: 7. In the sequence listing, the amino acid residues are consecutively numbered 1-31. Therefore, the first amino acid in wild type human GLP-1(7-37)—that is, the N-terminal histidine—is number “1” in SEQ ID NO: 7.

GLP-1 receptor agonist: The term “GLP-1 receptor agonist” may be used herein as a ligand which is capable of binding to the GLP-1 receptor and producing a biological response similar to that of the endogenous ligand, glucagon-like peptide 1 (GLP-1). A “full” GLP-1 receptor agonist may be defined as a GLP-1 receptor agonist which is capable of eliciting a biological response of the same magnitude as GLP-1.

Pharmaceutical composition: As used herein, the term “pharmaceutical composition” refers to a composition that is suitable for administration to a human or animal subject. In some embodiments, a pharmaceutical composition comprises one or more active agents formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and substantially pyrogen-free.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Treatment: The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic (preventative), palliative, symptomatic and/or curative.

### DETAILED DESCRIPTION

The present disclosure provides combinations, e.g., combination therapies, and/or compositions comprising an anti-ALK7 receptor antibody or antigen binding fragment thereof and a second biological active agent comprising (a) a GLP-1 receptor agonist, (b) an anti-GIP receptor antibody or antigen binding fragment thereof, and/or (c) an anti-Act receptor II antibody or antigen binding fragment thereof. The present disclosure also provides combinations, e.g., combination therapies, and/or compositions provided herein for use in prevention and/or treatment of diseases and/or disorders. In particular, the present disclosure provides an anti-ALK7 receptor antibody or antigen binding fragment thereof for use in prevention and/or treatment of overweight, obesity, and/or diabetes in combination with a second biological active agent.

### Anti-ALK7 Receptor Antibody or Antigen Binding Fragment Thereof

Activin Like Kinase 7 (ALK7) is a type I serine/threonine kinases, which forms a hexameric signaling complex on ligand binding. The signaling complex is a hetero oligomer that consists of a dimeric ligand, two type I and two type II transmembrane serine/threonine kinases. Type II receptors phosphorylate and activate type I receptors which autophosphorylate and then bind and activate the SMAD transcriptional regulators, SMAD2 and SMAD3. ALK7 is a receptor for activin B. ActB signaling via ALK7 in adipose tissue suppresses lipolysis, downregulates adrenergic receptors and facilitates fat accumulation, while inhibition of the ALK7 has been shown to reduce body weight and have other metabolic benefits in pre-clinical animal models.

In some embodiments, an anti-ALK7 receptor antibody or antigen binding fragment thereof specifically binds an ALK7 receptor. In some embodiments, an anti-ALK7 receptor antibody or antigen binding fragment thereof blocks the ALK7 receptor. In some embodiments, an anti-ALK7 receptor antibody or antigen binding fragment thereof is an anti-ALK7 receptor blocking antibody or antigen binding fragment thereof. In some embodiments, an anti-ALK7 receptor antibody or antigen binding fragment thereof is an ALK7 receptor antagonist, i.e., inhibits or blocks the ALK7 receptor. In some embodiments, an anti-ALK7 receptor antibody or antigen binding fragment thereof antagonizes the ALK7 receptor. In some embodiments, an anti-ALK7 receptor antibody or antigen binding fragment thereof inhibits ALK7 signaling. In some embodiments, an anti-ALK7 receptor antibody or antigen binding fragment thereof binds an ALK7 receptor and inhibit its biological activities, such as its ability to suppress lipolysis and/or prevent fat accumulation. In some embodiments, an anti-ALK7 receptor antibody or antigen binding fragment thereof increase lipolysis. In some embodiments, an anti-ALK7 receptor antibody or antigen binding fragment thereof is characterized in that it blocks formation of a hexameric signaling complex of an ALK7 receptor with an ActRIIA/B Type II receptor. In some embodiments, an anti-ALK7 receptor antibody or antigen binding fragment thereof inhibits SMAD2 and/or SMAD3 activation. In some embodiments, an anti-ALK7 receptor antibody or antigen binding fragment thereof is characterized in that it blocks binding of an ALK7 receptor ligand, such as activin B, GDF11, activin A, BMP10, BMP6, BMP5, GDF3, activin C, activin E, activin AC, activin BC, activin AE, nodal, or activin BE. In some embodiments, an anti-ALK7 receptor antibody or antigen binding fragment thereof is characterized in that it blocks binding of activin B to an ALK7 receptor. In some embodiments, an anti-ALK7 receptor antibody or antigen fragment thereof is characterized in that it blocks formation of a hexameric signaling complex of an ALK7 receptor with an ActRIIA/B Type II receptor.

The human ALK7 receptor gene (uniprotkb/Q8NER5) encodes a protein consisting of 493 amino acid residues, including a signal peptide, an extra-cellular domain, a transmembrane region and a cytoplasmic domain. The ALK7 receptor protein sequence is:

(SEQ ID NO: 1)
MTRALCSALRQALLLLAAAAELSPGLKCVCLLCDSSNFTCQTEGACWAS
VMLTNGKEQVIKSCVSLPELNAQVFCHSSNNVTKTECCFTDFCNNITLH
LPTASPNAPKLGPMELAIIITVPVCLLSIAAMLTVWACQGRQCSYRKKK
RPNVEEPLSECNLVNAGKTLKDLIYDVTASGSGSGLPLLVQRTIARTIV
LQEIVGKGRFGEVWHGRWCGEDVAVKIFSSRDERSWFREAEIYQTVMLR
HENILGFIAADNKDNGTWTQLWLVSEYHEQGSLYDYLNRNIVTVAGMIK
LALSIASGLAHLHMEIVGTQGKPAIAHRDIKSKNILVKKCETCAIADLG
LAVKHDSILNTIDIPQNPKVGTKRYMAPEMLDDTMNVNIFESFKRADIY
SVGLVYWEIARRCSVGGIVEEYQLPYYDMVPSDPSIEEMRKVVCDQKFR
PSIPNQWQSCEALRVMGRIMRECWYANGAARLTALRIKKTISQLCVKED
CKA.

In some embodiments, an anti-ALK7 receptor antibody or antigen binding fragment thereof binds the extracellular domain of an ALK7 receptor comprising the amino acid sequence 26-113 of SEQ ID NO: 1 or a portion thereof. In some embodiments, the extracellular domain (ECD) of an ALK7 receptor extends from the signal peptide to the transmembrane region i.e. aa 26-113 of the human ALK7 receptor coding region defined by SEQ ID NO: 1. In some embodiments, an ECD of ALK7 is shortened at the N-terminus including only amino acids 26-113 and is referred to as ALK7-ECD26-113 herein. In some embodiments, an anti-ALK7 receptor antibody or antigen binding fragment thereof binds human ALK7-ECD26-113.

In some embodiments, an anti-ALK7 receptor antibody or antigen binding fragment thereof comprises two variable regions. In some embodiments, an anti-ALK7 receptor antibody or antigen binding fragment thereof comprises an antigen binding domain comprising 6 CDR sequences. In some embodiments, an anti-ALK7 receptor antibody or antigen binding fragment thereof comprises two variable regions each comprising 3 CDR sequences each. In some embodiments, a variable region comprises several framework sequences. In some embodiments, a variable region sequence comprises FR1-CDR1-FR2-CDR2-FR3-CDR3-FR4 defined by e.g., Kabat, Chothia, and/or IMGT.

In some embodiments, CDRs are defined according to Kabat, Chothia, and/or IMGT. In some embodiments, CDRs are defined according to Kabat. In some embodiments, CDRs are defined according to IMGT. In some embodiments, CDRs are defined according to Chothia.

In some embodiments, an anti-ALK7 receptor antibody or antigen binding fragment thereof comprises a heavy chain and a light chain. In some embodiments, an anti-ALK7 receptor antibody or antigen binding fragment thereof comprises a variable heavy chain sequences and a variable light chain sequence. In some embodiments, an anti-ALK7 receptor antibody or antigen binding fragment thereof comprises a heavy and a light chain variable region each sequence comprising three CDRs. In some embodiments, an anti-ALK7 receptor antibody or antigen binding fragment thereof comprises a heavy chain variable sequence comprising three CDRs and a light chain variable sequence comprising three CDRs.

Exemplary anti-ALK7 antibodies amino acid sequences are included in Table A below.

TABLE A
Exemplary anti-ALK7 antibody related amino acid sequences

| SEQ ID NO: | Region | Amino acid sequence |
|---|---|---|
| 8 | HCDR1 | SGNYWN |
| 9 | HCDR1 | SGSYWN |
| 10 | HCDR2 | YISFDGRNNYNPSLKN |
| 11 | HCDR2 | YISFDGRTNYNPSLKN |
| 12 | HCDR2 | YISFDGRTNYNPSLKS |
| 13 | HCDR2 | YISFDGRRNYNPSLKN |
| 14 | HCDR2 | YISFTGRTNYNPSLKN |
| 15 | HCDR2 | YISFSGRNNYNPSLKN |
| 16 | HCDR2 | YISFSGRTNYNPSLKN |
| 17 | HCDR3 | DYYGSEGFGY |
| 18 | HCDR3 | DYYGSEGFAY |
| 19 | LCDR1 | KSSQSLLNSRTRKNYLA |
| 20 | LCDR1 | KSSQSLLVSRTRKNYLA |
| 21 | LCDR1 | KSSQSLLYSRTRKNYLA |
| 22 | LCDR2 | WTSTRES |
| 23 | LCDR2 | WASTRES |
| 24 | LCDR3 | KQSYNLPYT |
| 25 | LCDR3 | KRSYNLPYT |
| 43 | VH | QVQLQESGPGLVKPSESLSLTCTVEGYSITSGSYWNWIRQPPG |
| | | KGLEWIGYISFDGRTNYNPSLKNRVTISRDTSKNQFSLKLSSVT |
| | | AADTAVYYCARDYYGSEGFAYWGQGTLVTVSS |
| 78 | VL | DIVMTQSPDSLAVSLGEDATINCKSSQSLLVSRTRKNYLAWYQ |
| | | QKPGQPPKLLIYWTSTRESGVPDRFSGSGSGTDFTLTISSLQAE |
| | | DVAVYYCKQSYNLPYTFGGGTKLEIK |
| 85 | CH | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKRVEPKSCDKTHTCPPCPAPEAEGAPSVFLFPPKP |
| | | KDTLMISRTPEVTCVVVDVSHEDPEVKFNWYVDGVEVHNAKTK |
| | | PREEQYNSTYRVVSVLTVLHQDWLNGKEYKCKVSNKALPSSIE |
| | | KTISKAKGQPREPQVYTLPPSREEMTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 86 | CL | RTVAAPSVFIFPPSDEQLKSGTASVVCLLNNFYPREAKVQWKV |
| | | DNALQSGNSQESVTEQDSKDSTYSLSSTLTLSKADYEKHKVYA |
| | | CEVTHQGLSSPVTKSFNRGEC |
| 99 | Heavy | QVQLQESGPGLVKPSESLSLTCTVEGYSITSGSYWNWIRQPPG |
| | chain | KGLEWIGYISFDGRTNYNPSLKNRVTISRDTSKNQFSLKLSSVT |
| | | AADTAVYYCARDYYGSEGFAYWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSCDKTHTCPPCPAPEAEGAPSVFLFPPKPKDTLMISRTPE |
| | | VTCVVVDVSHEDPEVKFNWYVDGVEVHNAKTKPREEQYNSTY |
| | | RVVSVLTVLHQDWLNGKEYKCKVSNKALPSSIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMH |
| | | EALHNHYTQKSLSLSPGK |
| 100 | Light | DIVMTQSPDSLAVSLGEDATINCKSSQSLLVSRTRKNYLAWYQ |
| | chain | QKPGQPPKLLIYWTSTRESGVPDRFSGSGSGTDFTLTISSLQAE |
| | | DVAVYYCKQSYNLPYTFGGGTKLEIKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSFNR |
| | | GEC |

In some embodiments, the present disclosure provides an anti-ALK7 receptor antibody or antigen binding fragment thereof binding the extracellular domain of human ALK7 receptor (SEQ ID NO: 1) comprising three heavy chain CDR sequences, hCDR1-3, and three light chain CDR, sequences, ICDR1-3, wherein
a) hCDR1 is SGNYWN (SEQ ID NO: 8) with up to 1 or 2 amino acid substitutions,
b) hCDR2 is YISFDGRNNYNPSLKN (SEQ ID NO: 10) with up to 1, 2 or 3 amino acid substitutions,
c) hCDR3 is DYYGSEGFGY (SEQ ID NO: 17) with up to 1, 2 or 3 amino acid substitutions,
d) ICDR1 is KSSQSLLNSRTRKNYLA (SEQ ID NO: 19) with up to 1, 2 or 3 amino acid substitutions,
e) ICDR2 is WTSTRES (SEQ ID NO: 22) with up to 1 or 2 amino acid substitutions, and
f) ICDR3 is KQSYNLPYT (SEQ ID NO: 24) with up to 1 or 2 amino acid substitutions.

In some embodiments, the present disclosure provides an anti-ALK7 receptor antibody or antigen binding fragment thereof binding the extracellular domain of human ALK7 receptor (SEQ ID NO: 1) comprising three heavy chain CDR sequences, hCDR1-3, and three light chain CDR, sequences, ICDR1-3, wherein
a) hCDR1 is SGSYWN (SEQ ID NO: 9) with up to 1 or 2 amino acid substitutions,
b) hCDR2 is YISFDGRTNYNPSLKN (SEQ ID NO: 11) with up to 1, 2 or 3 amino acid substitutions,
c) hCDR3 is DYYGSEGFAY (SEQ ID NO: 18) with up to 1, 2 or 3 amino acid substitutions,
d) ICDR1 is KSSQSLLVSRTRKNYLA (SEQ ID NO: 20) with up to 1, 2 or 3 amino acid substitutions,
e) ICDR2 is WTSTRES (SEQ ID NO: 22) with up to1 or 2 amino acid substitutions, and
f) ICDR3 is KQSYNLPYT (SEQ ID NO: 24) with up to 1 or 2 amino acid substitutions.

In some embodiments, the present disclosure provides an anti-ALK7 receptor antibody or antigen binding fragment thereof binding the extracellular domain of human ALK7 receptor (SEQ ID NO: 1) comprising three heavy chain CDR sequences, hCDR1-3, and three light chain CDR, sequences, ICDR1-3, wherein

a) hCDR1 is
(SEQ ID NO: 9)
SGSYWN,
b) hCDR2 is
(SEQ ID NO: 11)
YISFDGRTNYNPSLKN,
c) hCDR3 is
(SEQ ID NO: 18)
DYYGSEGFAY,
d) lCDR1 is
(SEQ ID NO: 20)
KSSQSLLVSRTRKNYLA,
e) lCDR2 is
(SEQ ID NO: 22)
WTSTRES,
and
f) lCDR3 is
(SEQ ID NO: 24)
KQSYNLPYT.

In some embodiments, the present disclosure provides an anti-ALK7 receptor antibody or antigen binding fragment thereof binding the extracellular domain of human ALK7 receptor (SEQ ID NO: 1) comprising three heavy chain CDR sequences, hCDR1-3, and three light chain CDR, sequences, ICDR1-3, wherein
a) hCDR1 is SGNYWN (SEQ ID NO: 8) or SGSYWN (SEQ ID NO: 9) with up to 1 amino acid substitutions,
b) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid,
c) hCDR3 is DYYGSEGFGY (SEQ ID NO: 17) or DYYGSEGFAY (SEQ ID NO: 18) with up to 1 or 2 amino acid substitutions,
d) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
e) ICDR2 is WTSTRES (SEQ ID NO: 22) or WASTRES (SEQ ID NO: 23) with up to 1 amino acid substitutions, and
f) ICDR3 is KQSYNLPYT (SEQ ID NO: 24) or KRSYNLPYT (SEQ ID NO: 25) with up to 1 amino acid substitutions.

In some embodiments, an amino acid substitution is a conservative substitution or an alanine substitution. In some embodiments, an amino acid substitution is a conservative substitution. In some embodiments, an amino acid substitution is an alanine substitution.

In some embodiments, the present disclosure provides an anti-ALK7 receptor antibody or antigen binding fragment thereof binding the extracellular domain of human ALK7 receptor (SEQ ID NO: 1) comprising three heavy chain CDR sequences, hCDR1-3, and three light chain CDR, sequences, ICDR1-3, wherein

a) hCDR1 is
(SEQ ID NO: 8)
SGNYWN
or
(SEQ ID NO: 9)
SGSYWN,
b) hCDR2 is
(SEQ ID NO: 10)
YISFDGRNNYNPSLKN,
(SEQ ID NO: 11)
YISFDGRTNYNPSLKN,
(SEQ ID NO: 12)
YISFDGRTNYNPSLKS,
(SEQ ID NO: 13)
YISFDGRRNYNPSLKN,
(SEQ ID NO: 14)
YISFTGRTNYNPSLKN,
(SEQ ID NO: 15)
YISFSGRNNYNPSLKN
or
(SEQ ID NO: 16)
YISFSGRTNYNPSLKN
c) hCDR3 is
(SEQ ID NO: 17)
DYYGSEGFGY
or
(SEQ ID NO: 18)
DYYGSEGFAY,
d) lCDR1 is
(SEQ ID NO: 19)
KSSQSLLNSRTRKNYLA,
(SEQ ID NO: 20)
KSSQSLLVSRTRKNYLA,
or
(SEQ ID NO: 21)
KSSQSLLYSRTRKNYLA,
e) lCDR2 is
(SEQ ID NO: 22)
WTSTRES
or
(SEQ ID NO: 23)
WASTRES,
and
f) lCDR3 is
(SEQ ID NO: 24)
KQSYNLPYT
or
(SEQ ID NO: 25)
KRSYNLPYT.

In some embodiments, the present disclosure provides an anti-ALK7 receptor antibody or antigen binding fragment thereof binding the extracellular domain of human ALK7 receptor (SEQ ID NO: 1) comprising three heavy chain CDR sequences, hCDR1-3, and three light chain CDR, sequences, ICDR1-3, wherein
i. the three heavy chain CDR sequences are selected from the CDR sequences of any one of the heavy chain variable regions (VH) identified by SEQ ID NO: 26-59, and/or
ii. the three light chain CDR sequences are selected from the CDR sequences of any one of the light chain variable regions (VL) identified by SEQ ID NO: 60-84.

In some embodiments, an anti-ALK7 receptor antibody or antigen binding fragment thereof comprises a heavy chain variable region, a light chain variable region, or both. In some embodiments, an anti-ALK7 antibody or antigen binding fragment thereof comprises:
(a) a heavy chain variable region comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 43, and
(b) a light chain variable region comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 78.

In some embodiments, an anti-ALK7 antibody or antigen binding fragment thereof comprises:
(a) a heavy chain variable region comprising the amino acid sequence according to SEQ ID NO: 43, and
(b) a light chain variable region comprising the amino acid sequence according to SEQ ID NO: 78.

In some embodiments, an anti-ALK7 receptor antibody or antigen binding fragment thereof comprises a heavy chain constant region, a light chain constant region, or both. In some embodiments, an anti-ALK7 antibody or antigen binding fragment thereof comprises:
(a) a heavy chain constant region comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 85, and
(b) a light chain constant region comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 86.

In some embodiments, an anti-ALK7 antibody or antigen binding fragment thereof comprises:
(a) a heavy chain constant region comprising an amino acid sequence according to SEQ ID NO: 85, and
(b) a light chain constant region comprising an amino acid sequence according to SEQ ID NO: 86.

In some embodiments, an anti-ALK7 receptor antibody or antigen binding fragment thereof comprises a heavy chain, a light chain, or both. In some embodiments, an anti-ALK7 antibody or antigen binding fragment thereof comprises:
(a) a heavy chain comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 99, and
(b) a light chain comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to SEQ ID NO: 100.

In some embodiments, an anti-ALK7 antibody or antigen binding fragment thereof comprises:
(a) a heavy chain comprising an amino acid sequence according to SEQ ID NO: 99, and
(b) a light chain comprising an amino acid sequence according to SEQ ID NO: 100.

In some embodiments, an anti-ALK7 receptor antibody or antigen binding fragment thereof is or comprises an amino acid sequence having at least 70%, least 80%, at least 85%, at least 90%, at least 95%, at least 96%, at least 97%, at least 98%, at least 99% identity, or 100% identity to the amino acid sequence of QVQLVQSGAEVKKPGSSVKVSCKASGGTFSGQAISWVRQAPGQGLEWMGGIIPSFGTAR YAQKFQGRVTITADESTSTAYMELSSLRSEDTAVYYCARTPYYDSSGYLDVWGQGTMVTV SSASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKRVEPKSCDKTHTCPPCPAPEAE GAPSVFLFPPKPKDTLMISRTPEVTCVVVDVSHEDPEVKFNWYVDGVEVHNAKTKPREEQ YNSTYRVVSVLTVLHQDWLNGKEYKCKVSNKALPSSIEKTISKAKGQPREPQVYTLPPSR DELTKNQVSLTCLVKGFYPSDIAVEWESNGQPENNYKTTPPVLDSDGSFFLYSKLTVDKS RWQQGNVFSCSVMHEALHNHYTQKSLSLSPGKDIQMTQSPSSLSASVGDRVTITCQASHD IDNYLNWYQQKPGKAPKLLIYYASNLKTGVPSRFSGSGSGTDFTFTISSLQPEDIATYYCQQ SRASPPTFGGGTKVEIKRTVAAPSVFIFPPSDEQLKSGTASVVCLLNNFYPREAKVQWKVDN ALQSGNSQESVTEQDSKDSTYSLSSTLTLSKADYEKHKVYACEVTHQGLSSPVTKSFNRGE CQVQLVQSGAEVKKPGSSVKVSCKASGGTFSGQAISWVRQAPGQGLEWMGGIIPSFGTA RYAQKFQGRVTITADESTSTAYMELSSLRSEDTAVYYCARTPYYDSSGYLDVWGQGTMVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKRVEPKSCDKTHTCPPCPAPEA EGAPSVFLFPPKPKDTLMISRTPEVTCVVVDVSHEDPEVKFNWYVDGVEVHNAKTKPREE QYNSTYRVVSVLTVLHQDWLNGKEYKCKVSNKALPSSIEKTISKAKGQPREPQVYTLPPS RDELTKNQVSLTCLVKGFYPSDIAVEWESNGQPENNYKTTPPVLDSDGSFFLYSKLTVDK SRWQQGNVFSCSVMHEALHNHYTQKSLSLSPGKDIQMTQSPSSLSASVGDRVTITCQASH DIDNYLNWYQQKPGKAPKLLIYYASNLKTGVPSRFSGSGSGTDFTFTISSLQPEDIATYYCQ QSRASPPTFGGGTKVEIKRTVAAPSVFIFPPSDEQLKSGTASVVCLLNNFYPREAKVQWKV DNALQSGNSQESVTEQDSKDSTYSLSSTLTLSKADYEKHKVYACEVTHQGLSSPVTKSFNR GEC (SEQ ID NO: 6) or a fragment thereof. Bold font indicates two heavy chains. Italic font indicates two light chains.

In some embodiments, an anti-ALK7 receptor antibody or antigen binding fragment thereof is or comprises a heavy chain. In some embodiments, an anti-ALK7 receptor antibody or antigen binding fragment thereof heavy chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 97)
QVQLVQSGAEVKKPGSSVKVSCKASGGTFSGQAISWVRQAPGQGLEWMG
GIIPSFGTARYAQKFQGRVTITADESTSTAYMELSSLRSEDTAVYYCAR
TPYYDSSGYLDVWGQGTMVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPEAEGAPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPSSIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK.

In some embodiments, an anti-ALK7 receptor antibody or antigen binding fragment thereof heavy chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 99)
QVQLQESGPGLVKPSESLSLTCTVEGYSITSGSYWNWIRQPPGKGLEWI
GYISFDGRTNYNPSLKNRVTISRDTSKNQFSLKLSSVTAADTAVYYCAR
DYYGSEGFAYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPEAEGAPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPSSIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGK.

In some embodiments, an anti-ALK7 receptor antibody or antigen binding fragment thereof is or comprises a light chain. In some embodiments, an anti-ALK7 receptor antibody or antigen binding fragment thereof light chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 98)
DIQMTQSPSSLSASVGDRVTITCQASHDIDNYLNWYQQKPGKAPKLLIY
YASNLKTGVPSRFSGSGSGTDFTFTISSLQPEDIATYYCQQSRASPPTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, an anti-ALK7 receptor antibody or antigen binding fragment thereof is or comprises a light chain. In some embodiments, an anti-ALK7 receptor antibody or antigen binding fragment thereof light chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 100)
DIVMTQSPDSLAVSLGEDATINCKSSQSLLVSRTRKNYLAWYQQKPGQP
PKLLIYWTSTRESGVPDRFSGSGSGTDFTLTISSLQAEDVAVYYCKQSY
NLPYTFGGGTKLEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC.

In some embodiments, an anti-ALK7 receptor antibody or antigen binding fragment thereof is a Fab, a Fab′, a F(ab′) 2, a Fv, a Fd, a dAb, an scFv or a single domain antibody. In some embodiments, an anti-ALK7 receptor antibody or antigen binding fragment thereof is a full-length antibody. In some embodiments, an anti-ALK7 receptor antibody or antigen binding fragment thereof is an IgG antibody, such as an IgG1, IgG2, IgG3 or an IgG4 antibody. In some embodiments, an anti-ALK7 receptor antibody or antigen binding fragment thereof comprises heavy and light chain constant regions of an IgG sub type, such as any of IgG1, IgG2, IgG3 or an IgG4.

In some embodiments, a heavy chain constant region has the sequence of SEQ ID NO: 85 (wherein the C-terminal lysine is optional). In some embodiments, a light chain constant region has the sequence of SEQ ID NO: 86. In some embodiments, an anti-ALK7 receptor antibody or antigen binding fragment thereof comprises heavy and light chain constant regions including one or more amino acid substitutions to optimise Fc function. This may include amino acid substitution affecting cell interaction or effector protein interactions, such as FcR effector function or complement system effector functions, such as mutations equivalent to L117A, L118E, G120A, A213S, and P214S of SEQ ID NO: 85. Using the EU numbering system the mutations are referred to as L234A, L235E, G237A, A330S and/or P331S. In some embodiments, a constant region comprises one or more amino acid substitutions from the group consisting of L234A, L235E, G237A, A330S, P331S, or combinations thereof.

The present disclosure also provides methods of producing an anti-ALK7 antibody or antigen binding fragment thereof as provided herein. The method may entail cloning of an anti-ALK7 antibody in a suitable expression vector and establishing a host cell expressing such anti-ALK7 antibody. In some embodiments, the present disclosure provides a nucleotide sequence or pair of nucleotide sequences encoding an anti-ALK7 antibody or antigen binding fragment thereof as provided herein. In some embodiments, the present disclosure provides a host cell comprising a nucleotide sequence or pair of nucleotide sequences encoding an anti-ALK7 antibody or antigen binding fragment thereof as provided herein. In some embodiments, the present disclosure provides a host cell expressing an anti-ALK7 antibody or antigen binding fragment thereof as provided herein. In some embodiments, the present disclosure provides methods of producing an anti-ALK7 antibody or antigen binding fragment thereof comprising the steps of:
a) culturing a host cell expressing an anti-ALK7 antibody or antigen binding fragment thereof as provided herein; and
b) purifying said anti-ALK7 antibody or antigen binding fragment thereof.

Methods of preparing exemplary anti-ALK7 antibodies or antigen binding fragments thereof are described in Example 2 herein.

The in vitro potency of anti-ALK7 receptor antibodies or antigen binding fragments thereof on the ALK7 receptor may be measured as described in Example 3 herein. The potency of an anti-ALK7 receptor antibody or antigen binding fragment thereof may be described by means of its IC₅₀ values, wherein IC₅₀ is the concentration of an inhibitor (e.g., an anti-ALK7 receptor antibody or antigen binding fragment thereof) where the response is reduced by half. The lower the IC₅₀ value, the more potent the compound. In some embodiments, an anti-ALK7 receptor antibody or antigen binding fragment thereof as provided herein has an IC₅₀ value of less than 5 nM, such as less than 4 nM, such as less than 3 nM, such as less than 2.5 nM, such as less than 2 nM towards the human ALK7 receptor.

The affinity of an anti-ALK7 receptor antibody or antigen binding fragment thereof to its antigen can be determined by various methods, such as Surface Plasmon Resonance (SPR) wherein the kinetics of complex formation and dissociation is measured. In some embodiments, affinity (KD) for an anti-ALK7 receptor antibody or antigen binding fragment thereof is 1-1000 nM, such as 10-750 nM, or such as 10-500 nM. In some embodiments, the affinity (KD) for an anti-ALK7 receptor antibody or antigen binding fragment thereof is below 10 μM, such as below 5 μM, such as below 1 μM. In some embodiments, the affinity (KD) of an anti-ALK7 receptor antibody or antigen binding fragment thereof is below 100 nM, such as below 50 nM, such as below 10 nM.

### Second Biological Active Agent

The present disclosure provides combinations, e.g., combination therapies, and/or compositions comprising an anti-ALK7 receptor antibody or antigen binding fragment thereof and a second biological active agent. The present disclosure provides an anti-ALK7 receptor antibody or antigen binding fragment thereof for use in prevention and/or treatment of overweight, obesity, and/or diabetes in combination with a second biological active agent.

In some embodiments, a second biological active agent is or comprises one or more agonists, such as receptor agonists. In some embodiments, a second biological active agent is or comprises one or more antagonists, such as one or more receptor antagonists. In some embodiments, a second biological active agent is or comprises one or more antibodies or antigen binding fragments thereof. In some embodiments, a second biological active agent is an incretin hormone or a variant thereof.

In some embodiments, a second biological active agent is or comprises a GLP-1 receptor agonist.

In some embodiments, a second biological active agent is or comprises a GLP-1 receptor-amylin receptor agonist.

In some embodiments, a second biological active agent is or comprises a GIP receptor antagonist, e.g., an anti-GIP receptor antibody or antigen binding fragment thereof.

In some embodiments, a second biological active agent is or comprises an ActRII antagonist, e.g., an anti-ActRII antibody or antigen binding fragment thereof.

### GLP-1 Receptor Agonist

Glucagon-like peptide 1 (GLP-1) is a gut enteroendocrine cell-derived hormone and one of two prominent endogenous physiological incretins. GLP-1 improves glycemic control by stimulating glucose-dependent insulin secretion in response to nutrients (glucose), inhibits glucagon secretion from the pancreatic alpha-cells, slows gastric emptying, and induces body weight loss primary by decreasing food consumption.

In some embodiments, a second biological active agent activates a GLP-1 receptor. In some embodiments, a second biological active agent is or comprises a GLP-1 receptor agonist, i.e., a GLP-1 analogue. In some embodiments, combinations, e.g., a combination therapies, and/or compositions comprises an anti-ALK7 receptor antibody or antigen binding fragment thereof and a GLP-1 receptor agonist.

In some embodiments, the present disclosure provides an anti-ALK7 receptor antibody or antigen binding fragment thereof for use in prevention and/or treatment of overweight, obesity, and/or diabetes in combination with a GLP-1 receptor agonist. In some embodiments, a GLP-1 receptor agonist is characterized in that it binds and activates, or “agonises” a GLP-1 receptor. In other words, in some embodiments, a GLP-1 receptor agonist is “potent” on the GLP-1 receptor. The in vitro potency of a GLP-1 receptor agonist on the GLP-1 receptor may be measured as described in WO/2022/129526, Assay 1. The potency of a GLP-1 receptor agonist may be described by means of its EC₅₀ values, wherein EC₅₀ represents the concentration of compound upon which 50% of its maximal effect is observed. The lower the EC₅₀ value, the more potent the compound. In some embodiments, a GLP-1 receptor agonist as provided herein has an EC₅₀ value of less than 300 pM, such as less than 200 pM, such as less than 150 pM, preferably less than 100 pM, such as less than 75 pM, even more preferably less than 50 pM, such as less than 40 pM, such as less than 30 pM, such as less than 20 pM, such as less than 13 pM towards to human GLP-1 receptor.

Examples of exogenous GLP-1 receptor agonists include semaglutide (the active pharmaceutical ingredient in Ozempic®, Rybelsus®, and Wegovy®), liraglutide (the active pharmaceutical ingredient in Victoza® and Saxenda®), tirzepatide (the active pharmaceutical ingredient in Mounjaro® and Zepbound®) and dulaglutide (the active pharmaceutical ingredient in Trulicity®).

Exemplary GLP-1 agonists are provided in WO2006/097537 and WO/2022/129526. WO2006/097537 and WO/2022/129526 are herein incorporated by reference.

In some embodiments, a GLP-1 receptor agonist comprises an unnatural amino acid. In some embodiments, an unnatural amino acid is alpha(α)-aminoisobutyryl, α-aminoisobutyric acid or α-methylalanine herein abbreviated as Aib. In some embodiments, alpha-aminoisobutyryl is located in the N-terminal part of the GLP-1 sequence. In some embodiments, a GLP-1 receptor agonist is or comprises an amino acid sequence having at least 70%, least 80%, at least 85%, at least 90%, at least 95%, at least 96%, at least 97%, at least 98%, at least 99% identity, or 100% identity to the amino acid sequence of
H-Aib-EGTFTSDVSSYLEGQAAKEFIAWLVRGRG (SEQ ID NO: 2). As noted previously, Aib is 2-aminoisobutyric acid.

In some embodiments, a GLP-1 receptor agonist comprises 1-3 protraction moieties. In some embodiments, a protraction moiety is covalently bound to lysine (K) residue at position of SEQ ID NO: 2. In some embodiments, a GLP-1 receptor agonist comprises a fatty acid protractor. In some embodiments, a fatty acid protractor is a C16-C20 diacid, e.g., a C18 diacid.

In some embodiments, a GLP-1 receptor agonist comprises or is according to formula I (SEQ ID NO: 106) below

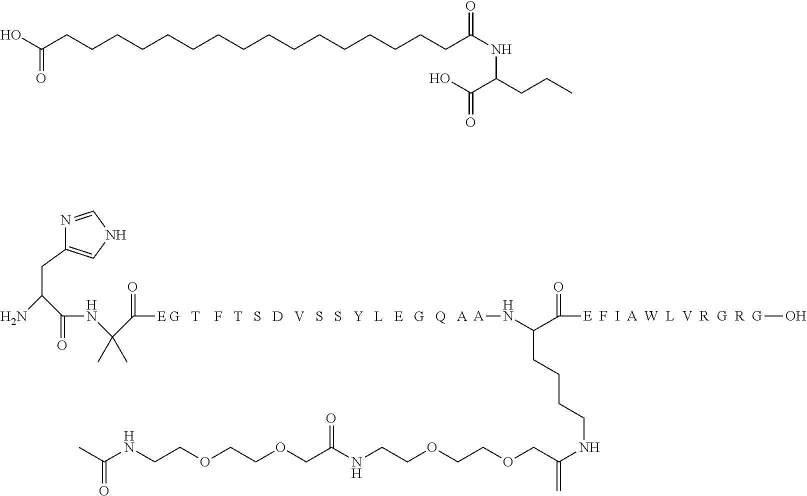

(Formula I, SEQ ID NO: 106) or a pharmaceutically acceptable salt thereof.

In some embodiments, a GLP-1 receptor agonist is or comprises semaglutide (an exemplary GLP-1 receptor agonist). In some embodiments, a GLP-1 receptor agonist is N^{6.26}-{18-[N-(17-carboxyheptadecanoyl)-L-γ-glutamyl]-10-oxo-3,6, 12, 15-tetraoxa-9, 18-diazaoctadecanoyl}-[8-(2-amino-2-propanoic acid), 34-L-arginine]human glucagon-like peptide 1(7-37). Semaglutide is described in WO2006/097537 and in J. Med. Chem. 2015, 58, 18, 7370-7380 and may be manufactured using methods well known to the person skilled in the art, such as that briefly described in WO2006/097537, Example 4.

In some embodiments, a GLP-1 receptor agonist comprises or is according to formula II (SEQ ID NO: 107) below

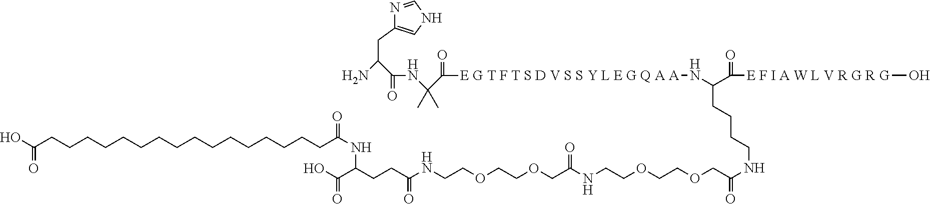

(Formula II, SEQ ID NO: 107) or a pharmaceutically acceptable salt thereof.

In some embodiments, semaglutide is present in its fully or partly ionised form. In some embodiments, one or more carboxylic acid groups (—COOH) of semaglutide may be deprotonated into the carboxylate group (—COO—) and/or one or more amino groups (—NH₂) may be protonated into the —NH₃⁺ group. In some embodiments, semaglutide may be in the form of a salt, preferably a pharmaceutically acceptable salt.

### GLP-1 Receptor-Amylin Receptor Agonist

In some embodiments, a second biological active agent is or comprises an amylin receptor agonist, such as an amylin receptor 3 agonist. In some embodiments, a second biological active agent comprises two or more agonists. In some embodiments, a second biological active agent is or comprises a GLP-1 receptor agonist and an amylin receptor agonist, e.g., an amylin receptor 3 agonist. In some embodiments, a second biological active agent is or comprises a GLP-1 receptor-amylin receptor agonist. In some embodiments, a GLP-1 receptor-amylin receptor agonist is a single molecule.

In some embodiments, an amylin receptor agonist activates and/or agonises a calcitonin receptor (CTR) and/or an amylin receptor (AMYR). Amylin receptors consist of heterodimers of two components: the calcitonin receptor (CTR) and one of three receptor activity-modifying proteins (RAMP1-3), resulting in three possible complexes, AMYR1-3. Unless otherwise specified herein, “amylin receptor” at least refers to amylin receptor 3 (AMYR3). In some embodiments, a second biological active agent comprising an amylin receptor agonist has cross-reactivity to a amylin receptor and a calcitonin receptor. In some embodiments, a second biological active agent comprising an amylin receptor agonist activates and/or agonises an amylin receptor and does not have cross-reactivity to a calcitonin receptor. In some embodiments, second biological active agents provided herein comprise an amylin receptor agonist. Examples of endogenous amylin receptor agonists are human amylin and human calcitonin. Examples of exogenous amylin receptor agonists are pramlintide and cagrilintide as described in disclosed in WO2012/168432, herein incorporated by reference.

In some embodiments, a GLP-1 receptor-amylin receptor agonist is or comprises a peptide. In some embodiments, a GLP-1 receptor-amylin receptor agonist is or comprises a single chain polypeptide backbone comprising one, two or three lysine (Lys, K) residues. In some embodiments, a GLP-1 receptor-amylin receptor agonist is or comprises a single chain polypeptide backbone. In some embodiments, a GLP-1 receptor-amylin receptor agonist may further comprise 1-3 protraction moieties. A protraction moiety may be covalently bound to lysine (Lys, K) residue(s).

In some embodiments, an amide moiety at the C-terminus of the amylin receptor agonist is free in order for the amylin receptor agonist to retain maximal bioactivity. Hence, in some embodiments, the C-terminal residue of a GLP-1 receptor agonist is covalently linked to the N-terminal residue of either an optional peptide linker or the amylin receptor agonist and the N-terminal residue of the amylin receptor agonist is covalently linked to the C-terminal of either an optional peptide linker or the GLP-1 receptor agonist. In some embodiments, a peptide comprises a linker. In some embodiments, a linker is a peptide linker. In some embodiments, a linker comprises 1-30 naturally occurring amino acids, such as 3-6 amino acids, such as 4-5 amino acids, such as 5 amino acids.

In some embodiments, a polypeptide backbone of the GLP-1 receptor-amylin receptor agonist comprises 1, 2 or 3 lysine residues. In some embodiments, a polypeptide backbone of the GLP-1 receptor-amylin receptor agonist comprises 1 or 2 lysine residues. In some embodiments, a polypeptide backbone of a GLP-1 receptor-amylin receptor agonist comprises 1 lysine residue.

In some embodiments, a GLP-1 receptor-amylin receptor agonist comprises a GLP-1 receptor agonist linked to a amylin receptor agonist. In some embodiments, a linker is or comprises one or more glycine residues. In some embodiments, a linker comprises 1-6 glycine residues (SEQ ID NO: 101). In some embodiments, a linker is or comprises four glycine residues (SEQ ID NO: 102). In some embodiments, a linker is a GGGGE linker (SEQ ID NO: 103). In some embodiments, a linker is a γGlu-2xOEG linker.

In some embodiments, a polypeptide backbone of a GLP-1 receptor-amylin receptor agonist provided herein may be referred to as “R1” and described by Formula III: Z1-Z2-Z3, wherein Z1 is a GLP-1 receptor agonist peptide, Z2 is an optional peptide linker and Z3 is an amylin receptor agonist peptide. In some embodiments, Z1 comprises a maximum of 9 amino acid modifications relative to wild type GLP-1(7-37) (SEQ ID NO: 7). In some embodiments, the C-terminus of Z1 is attached either to Z2, when Z2 is present, or to Z3, when Z2 is absent. In some embodiments, Z2 is a peptide linker. In some embodiments, when Z2 is present, its N-terminus is attached to the C-terminus of Z1 and its C-terminus is attached to the N-terminus of Z3. In some embodiments, Z1 is or comprises H-Aib-EGTFTSDVSSYLEEQAAREFIAWLVRGRK (SEQ ID NO: 91). In some embodiments, an amylin receptor agonist comprises a peptide “Z3”. In some embodiments, an amylin receptor agonist comprises a C-terminal amide. Without being bound by a particular theory, the C-terminal amide might be important for amylin receptor agonist bioactivity. In some embodiments, Z3 comprises a maximum of 7 amino acid modifications, relative to SEQ ID NO: 92 (ASELSTAALGRLSAELHELATLPRTETGSGSP). In some embodiments, the C-terminus of Z3 is modified with an amide group. In some embodiments, the N-terminus of Z3 is attached to the C-terminus of Z2, when Z2 is present, or to the C-terminus of Z1, when Z2 is absent. In some embodiments, Z1-Z2-Z3 comprises one, two or three lysine and/or cysteine residues. In some embodiments, each lysine and/or cysteine residue is covalently bound to a protraction moiety, which may be referred to herein as “R2-R3”, wherein “R2” is an optional linker and “R3” is a protractor.

In some embodiments, a polypeptide backbone of a duel GLP-1 receptor-amylin receptor agonist is or comprises an amino acid sequence having at least 70%, least 80%, at least 85%, at least 90%, at least 95%, at least 96%, at least 97%, at least 98%, at least 99% identity, or 100% identity to amino acid the sequence of H-Aib-EGTFTSDVSSYLEEQAAREFIAWLVRGRKGGGGEASELSTAALGRLSAELHELATLPRTETG SGSP (SEQ ID NO: 3). In some embodiments, a GLP-1 receptor-amylin receptor agonist comprises an unnatural amino acid 2-aminoisobutyric acid in the N-terminal part of the GLP-1 sequence. In some embodiments, a GLP-1 receptor-amylin receptor agonist is acylated at a lysine residue in the GLP-1 receptor agonist portion. In some embodiments, a GLP-1 receptor-amylin receptor agonist comprises a C16 diacid attached via a linker. In some embodiments, a GLP-1 receptor-amylin receptor agonist comprises a C18 diacid attached via a linker. In some embodiments, a GLP-1 receptor-amylin receptor agonist comprises a C20 diacid attached via a linker. In some embodiments, a GLP-1 receptor-amylin receptor agonist comprises a C22 diacid attached via a linker. In some embodiments, a linker (R2) is a γGlu-2xOEG linker. In some embodiments, a duel GLP-1 receptor-amylin receptor agonist is or comprises H-Aib-EGTFTSDVSSYLEEQAAREFIAWLVRGR-K([2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl])-GGGGEASELSTAALGRLSAELHELATLPRTETGSGSP-amide (SEQ ID NO: 104).

In some embodiments, a duel GLP-1 receptor-amylin receptor agonist comprises or is according to Formula IV (SEQ ID NO: 108):

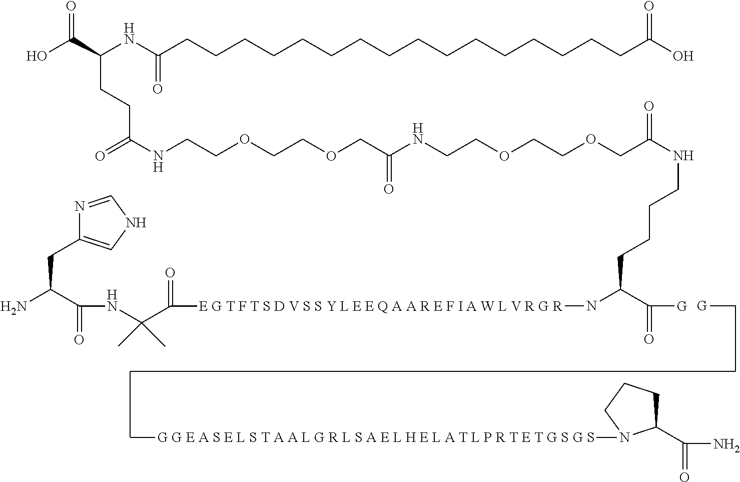

or a pharmaceutically acceptable salt thereof.

In some embodiments, a duel GLP-1 receptor-amylin receptor agonist is or comprises H-Aib-EGTFTSDVSSYLEEQAAREFIAWLVRGR-K([2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl])-GGGGEASELSTAALGRLSAELHELATLPRTETGSGSP-amide (SEQ ID NO: 105).

In some embodiments, a duel GLP-1 receptor-amylin receptor agonist comprises or is according to Formula V (SEQ ID NO: 109):

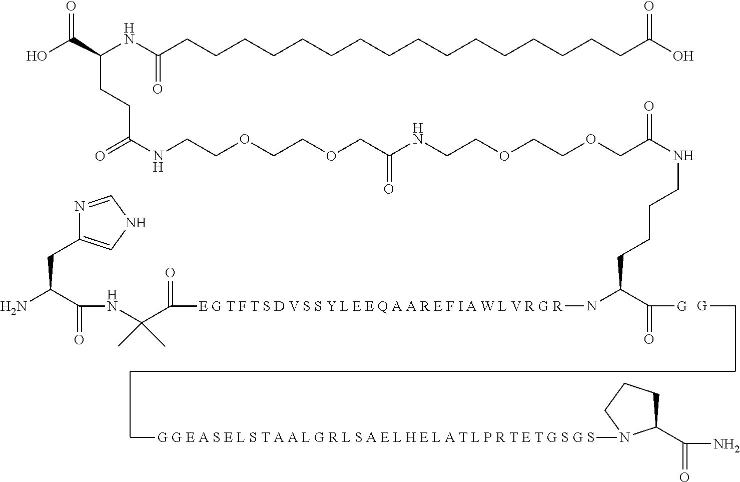

or a pharmaceutically acceptable salt thereof.

In some embodiments, a GLP-1 receptor-amylin receptor agonist exhibits a variety of properties rendering it useful as a medicament, as described herein.

In some embodiments, a GLP-1 receptor-amylin receptor agonist is potent on the GLP-1 receptor and on the amylin receptor, such as amylin receptor 3. In vitro potency of a GLP-1 receptor-amylin receptor agonist on the GLP-1 receptor and on the amylin-3 receptor may be measured as described in Assays 1 (Human GLP-1 receptor in vitro potency assay) and 2 (Human amylin receptor in vitro potency assay) in WO/2022/129526, respectively. WO/2022/129526 is herein incorporated by reference. The potency of the compound may be described by means of its EC₅₀ values. EC₅₀ represents the concentration of compound upon which 50% of its maximal effect is observed. The lower the EC₅₀ value, the more potent the compound. In some embodiments, a GLP-1 receptor-amylin receptor agonist as provided herein has an EC₅₀ value of less than 200 pM, such as less than 150 pM, preferably less than 100 pM, such as less than 75 pM, even more preferably less than 50 pM, such as less than 40 pM, such as less than 30 pM, such as less than 20 pM, such as less than 10 pM, such as less than 6 towards the human GLP-1 receptor. In some embodiments, a GLP-1 receptor-amylin receptor agonist as provided herein has an EC₅₀ value of less than 200 pM, such as less than 150 pM, preferably less than 100 pM, such as less than 75 pM, even more preferably less than 50 pM, such as less than 40 pM, such as less than 30 pM, such as less than 20 pM, such as less than 10 pM, such as less than 5 towards the human amylin receptor 3.

In some embodiments, a GLP-1 receptor-amylin receptor agonist administered in combination with an anti-ALK7 antibody or antigen binding fragment thereof to a subject is useful in total body weight loss, such as fat weight loss while preventing lean mass loss.

### Anti-GIP Receptor Antibody or Antigen Binding Fragment Thereof

Glucose-dependent insulinotropic polypeptide (GIP), another prominent incretin besides GLP-1, improves glycemic control by stimulation of insulin secretion in response to nutrients (fat, glucose). Furthermore, GIP appears to improve plasma lipid profile and to stimulate calcium accumulation in bones. In contrast to GLP-1, the incretin effect of GIP is severely reduced in type 2 diabetes patients, though recent studies suggest that GIP efficiency can be regained in these patients after treatment to improve glucose control. Nonetheless, the role of GIP to regulate systemic metabolism beyond its direct effect on the endocrine pancreas remains controversial, particularly as it relates to GIP action to promote gain in fat mass in animal models.

The present disclosure provides combinations, e.g., combination therapies, and/or compositions comprising an anti-ALK7 receptor antibody or antigen binding fragment thereof and an anti-GIP receptor antibody or antigen binding fragment thereof. The present disclosure also provides an anti-ALK7 receptor antibody or antigen binding fragment thereof for use in prevention and/or treatment of overweight, obesity, and/or diabetes in combination with an anti-GIP receptor antibody or antigen binding fragment thereof.

In some embodiments, a second biological active agent is or comprises an anti-GIP receptor antibody or antigen binding fragment thereof. In some embodiments, an anti-GIP receptor antibody or antigen binding fragment thereof binds a GIP receptor. In some embodiments, an anti-GIP receptor antibody or antigen binding fragment thereof is characterized in that it prevents binding of GIP to the GIP receptor. In other words, an anti-GIP receptor antibody or antigen binding fragment thereof is characterized in that it binds to the GIP receptor without activating the GIP receptor or reducing the activation compared to the native ligand, such as GIP. In some embodiments, an anti-GIP receptor antibody or antigen binding fragment thereof is an antagonistic anti-GIP receptor antibody.

In some embodiments, an anti-GIP receptor antibody or antigen binding fragment thereof is or comprises an amino acid sequence having at least 70%, least 80%, at least 85%, at least 90%, at least 95%, at least 96%, at least 97%, at least 98%, at least 99% identity, or 100% identity to the amino acid sequence of QSLEESGGDLVRPEASLTLTCTASGFSFYTNYFLCWVRQAPGKGLEWIACIYIGAGGDTYY ASWAKGRFTISKTSSTTVTLQMTSLTDADTATYFCTRYVNILDYWFHDLWGQGTLVTVSSA STKGPSVFPLAPCSRSTSESTAALGCLVKDYFPEPVTVSWNSGALTSGVHTFPAVLQSSG LYSLSSVVTVPSSSLGTKTYTCNVDHKPSNTKVDKRVESKYGPPCPPCPAPEFLGGPSVFL FPPKPKDTLMISRTPEVTCVVVDVSQEDPEVQFNWYVDGVEVHNAKTKPREEQFNSTYRV VSVLTVLHQDWLNGKEYKCKVSNKGLPSSIEKTISKAKGQPREPQVYTLPPSQEEMTKNQ VSLTCLVKGFYPSDIAVEWESNGQPENNYKTTPPVLDSDGSFFLYSRLTVDKSRWQEGNV FSCSVMHEALHNHYTQKSLSLSLGKQSLEESGGDLVRPEASLTLTCTASGFSFYTNYFLC WVRQAPGKGLEWIACIYIGAGGDTYYASWAKGRFTISKTSSTTVTLQMTSLTDADTATYFC TRYVNILDYWFHDLWGQGTLVTVSSASTKGPSVFPLAPCSRSTSESTAALGCLVKDYFPE PVTVSWNSGALTSGVHTFPAVLQSSGLYSLSSVVTVPSSSLGTKTYTCNVDHKPSNTKVD KRVESKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLPSSIEKTI SKAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGKDVVMTQTP ASVSESVGGTVTIKCQASQTIGSNLAWYQQKPGQRPKLLIYGASYLASGVPSRFKGSGSGT EYTLTISDLECADAATYYCQCTYTNNNYVGFGGGTEVVVKRTVAAPSVFIFPPSDEQLKSGT ASVVCLLNNFYPREAKVQWKVDNALQSGNSQESVTEQDSKDSTYSLSSTLTLSKADYEKHK VYACEVTHQGLSSPVTKSFNRGECDVVMTQTPASVSESVGGTVTIKCQASQTIGSNLAWYQ QKPGQRPKLLIYGASYLASGVPSRFKGSGSGTEYTLTISDLECADAATYYCQCTYTNNNYVG FGGGTEVVVKRTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSFNRGEC (SEQ ID NO: 4) or a fragment thereof. Bold font indicates two heavy chains. Italic font indicates two light chains.

In some embodiments, an anti-GIP receptor antibody or antigen binding fragment thereof is or comprises a heavy chain. In some embodiments, an anti-GIP receptor antibody or antigen binding fragment thereof heavy chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 93)
QSLEESGGDLVRPEASLTLTCTASGFSFYTNYFLCWVRQAPGKGLEWIA
CIYIGAGGDTYYASWAKGRFTISKTSSTTVTLQMTSLTDADTATYFCTR
YVNILDYWFHDLWGQGTLVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKS
LSLSLGK.

In some embodiments, an anti-GIP receptor antibody or antigen binding fragment thereof is or comprises a light chain. In some embodiments, an anti-GIP receptor antibody or antigen binding fragment thereof light chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 94)
DVVMTQTPASVSESVGGTVTIKCQASQTIGSNLAWYQQKPGQRPKLLIY
GASYLASGVPSRFKGSGSGTEYTLTISDLECADAATYYCQCTYTNNNYV
GFGGGTEVVVKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC.

The in vitro potency of an anti-GIP receptor antibody or antigen binding fragment thereof on the GIP receptor may be measured as described in Example 4. The potency of an anti-GIP receptor antibody or antigen binding fragment thereof may be described by means of its IC₅₀ values, wherein IC₅₀ is the concentration of an inhibitor (e.g., an anti-GIP receptor antibody or antigen binding fragment thereof) where the response is reduced by half. The lower the IC₅₀ value, the more potent the compound. In some embodiments, an anti-GIP receptor antibody or antigen binding fragment thereof as provided herein has an IC₅₀ value of less than 300 nM, such as less than 250 nM, such as less than 200 nM, such as less than 150 nM, such as less than 112 nM towards the human GIP receptor.

### Anti-Activin (Act) Receptor Type II Antibody or Antigen Binding Fragment Thereof

Activin Type II receptors (ActIIR), such as ActRIIA and ActRIIB, play a role in muscle growth and fat regulation. Myostatin and activin A are proteins that negatively regulate muscle growth by promoting protein degradation resulting in muscle atrophy. Therefore, blocking of myostatin and activin A signals promotes protein synthesis resulting in muscle growth and reduces fat mass. Increasing muscle mass is highly beneficial for improving overall metabolic health and overall wellbeing. Skeletal muscle is necessary for movement and is an energetically costly tissue for body to maintain, increase muscle mass results in increased basal metabolic rate with can protect against gaining fat mass. In addition, skeletal muscle is the major site of insulin stimulated glucose disposal, which and as a result is negatively impacted by type 2 diabetes. As a result, it is imperative identify mechanisms to maintain/increase skeletal muscle in order improve overall health.

The present disclosure provides combinations, e.g., combination therapies, and/or compositions comprising an anti-ALK7 receptor antibody or antigen binding fragment thereof and an anti-ActRII antibody or antigen binding fragment thereof. The present disclosure also provides an anti-ALK7 receptor antibody or antigen binding fragment thereof for use in prevention and/or treatment of overweight, obesity, and/or diabetes in combination with an anti-ActRII antibody or antigen binding fragment thereof.

In some embodiments, a second biological active agent is or comprises an anti-ActRII antibody or antigen binding fragment thereof. In some embodiments, an anti-ActRII antibody or antigen binding fragment thereof binds an ActR, e.g., an ActRII. In some embodiments, an anti-ActRII antibody or antigen binding fragment thereof is characterized in that it prevents binding of myostatin and/or activin A to an ActRII. In other words, an anti-ActRII antibody or antigen binding fragment thereof is characterized in that it binds to the ActRII without activating the ActRII or reducing the activation compared to the native ligand, such as myostatin and/or activin A. In some embodiments, an anti-ActRII antibody or antigen binding fragment thereof is an antagonistic anti-ActRII antibody.

In some embodiments, an anti-ActRII antibody or antigen binding fragment thereof is or comprises an amino acid sequence having at least 70%, least 80%, at least 85%, at least 90%, at least 95%, at least 96%, at least 97%, at least 98%, at least 99% identity, or 100% identity to the amino acid sequence of QSALTQPASVSGSPGQSITISCTGTSSDVGSYNYVNWYQQHPGKAPKLMIYGVSKRPSGVS NRFSGSKSGNTASLTISGLQAEDEADYYCGTFAGGSYYGVFGGGTKLTVLGQPKAAPSVTL FPPSSEELQANKATLVCLISDFYPGAVTVAWKADSSPVKAGVETTTPSKQSNNKYAASSYLS LTPEQWKSHRSYSCQVTHEGSTVEKTVAPTECSQSALTQPASVSGSPGQSITISCTGTSSD VGSYNYVNWYQQHPGKAPKLMIYGVSKRPSGVSNRFSGSKSGNTASLTISGLQAEDEADY YCGTFAGGSYYGVFGGGTKLTVLGQPKAAPSVTLFPPSSEELQANKATLVCLISDFYPGAVT VAWKADSSPVKAGVETTTPSKQSNNKYAASSYLSLTPEQWKSHRSYSCQVTHEGSTVEKT VAPTECSQVQLVQSGAEVKKPGASVKVSCKASGYTFTSSYINWVRQAPGQGLEWMGTIN PVSGSTSYAQKFQGRVTMTRDTSISTAYMELSRLRSDDTAVYYCARGGWFDYWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKRVEPKSCDKTHTCPPCPAPEA AGGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLPPS REEMTKNQVSLTCLVKGFYPSDIAVEWESNGQPENNYKTTPPVLDSDGSFFLYSKLTVDK SRWQQGNVFSCSVMHEALHNHYTQKSLSLSPGKQVQLVQSGAEVKKPGASVKVSCKAS GYTFTSSYINWVRQAPGQGLEWMGTINPVSGSTSYAQKFQGRVTMTRDTSISTAYMELSR LRSDDTAVYYCARGGWFDYWGQGTLVTVSSASTKGPSVFPLAPSSKSTSGGTAALGCLV KDYFPEPVTVSWNSGALTSGVHTFPAVLQSSGLYSLSSVVTVPSSSLGTQTYICNVNHKPS NTKVDKRVEPKSCDKTHTCPPCPAPEAAGGPSVFLFPPKPKDTLMISRTPEVTCVVVDVS HEDPEVKFNWYVDGVEVHNAKTKPREEQYNSTYRVVSVLTVLHQDWLNGKEYKCKVSNK ALPAPIEKTISKAKGQPREPQVYTLPPSREEMTKNQVSLTCLVKGFYPSDIAVEWESNGQP ENNYKTTPPVLDSDGSFFLYSKLTVDKSRWQQGNVFSCSVMHEALHNHYTQKSLSLSPG K (SEQ ID NO: 5) or a fragment thereof. Bold font indicates two heavy chains. Italic font indicates two light chains.

In some embodiments, an anti-ActRII antibody or antigen binding fragment thereof is or comprises a heavy chain. In some embodiments, an anti-ActRII antibody or antigen binding fragment thereof heavy chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 95)
QVQLVQSGAEVKKPGASVKVSCKASGYTFTSSYINWVRQAPGQGLEWMG
TINPVSGSTSYAQKFQGRVTMTRDTSISTAYMELSRLRSDDTAVYYCAR
GGWFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPGK.

In some embodiments, an anti-ActRII antibody or antigen binding fragment thereof is or comprises a light chain. In some embodiments, an anti-ActRII antibody or antigen binding fragment thereof light chain is or comprises an amino acid sequence having at least 70%, least 80%, at least 85%, at least 90%, at least 95%, at least 96%, at least 97%, at least 98%, at least 99% identity, or 100% identity to the amino acid sequence of

(SEQ ID NO: 96)
QSALTQPASVSGSPGQSITISCTGTSSDVGSYNYVNWYQQHPGKAPKLM
IYGVSKRPSGVSNRFSGSKSGNTASLTISGLQAEDEADYYCGTFAGGSY
YGVFGGGTKLTVLGQPKAAPSVTLFPPSSEELQANKATLVCLISDFYPG
AVTVAWKADSSPVKAGVETTTPSKQSNNKYAASSYLSLTPEQWKSHRSY
SCQVTHEGSTVEKTVAPTECS.

### Uses and Methods

The present disclosure provides anti-ALK7 receptor antibodies or antigen binding fragments thereof for use as a medicament in combination with a second biological active agent, such as, but not limited to, a GLP-1 receptor agonist; GLP-1 receptor-amylin receptor agonist; an anti-GIP receptor antibody or antigen binding fragment thereof; and/or anti-ActRII antibody or antigen binding fragment thereof.

In some embodiments, an anti-ALK7 receptor antibody or antigen binding fragment thereof is administered together with a second biological active agent. In some embodiments, a combination comprising an anti-ALK7 receptor antibody or antigen binding fragment thereof and a second biological active agent is administered in a co-formulation. In some embodiments, an anti-ALK7 receptor antibody or antigen binding fragment thereof is administered concomitantly or sequentially with the second biological active agent. In some embodiments, a combination comprising an anti-ALK7 receptor antibody or antigen binding fragment thereof and a second biological active agent is administered in a loose combination.

In some embodiments, the present disclosure provides combination therapies. In some embodiments, a combination therapy comprises administration of anti-ALK7 receptor antibodies or antigen binding fragments thereof in combination with a second biological active agent to a subject in need thereof. In some embodiments, the present disclosure provides an anti-ALK7 antibody or antigen binding fragment thereof in combination with a second biological active agent for use as a medicament.

In some embodiments, combinations, e.g., combination therapies, compositions, and/or pharmaceutical compositions disclosed herein are for medical use. Combination therapies described herein can be provided in various forms, e.g., in the form of a loose combination or a co-formulation. Provided herein are methods of treating or preventing of overweight, obesity, and/or diabetes. In some embodiments, diabetes is type 2 diabetes.

In some embodiments, the present disclosure provides (i) an anti-ALK7 receptor antibody or antigen binding fragment thereof and (ii) a second biological active agent as provided herein for use in prevention and/or treatment of overweight, obesity, and/or diabetes.

In some embodiments, a method of preventing and/or treating overweight, obesity, and/or diabetes comprises:
(i) administering an effective amount of anti-ALK7 receptor antibody or antigen binding fragment thereof, and
(ii) administering an effective amount of a second biological active agent as provided herein.

Uses according to the present disclosure may comprise administration of a therapeutically effective dose of an anti-ALK7 receptor antibody or antigen binding fragment thereof and a second biological active agent. Combinations and/or compositions are administered to a patient in an amount sufficient to substantially exert their biological function, e.g., inhibit the ALK7 receptor, activate the GLP-1 receptor, and/or amylin receptor, as described above. An amount adequate to accomplish this is defined as a “therapeutically effective dose”, which may provide for a weight loss, prevention, and/or treatment of obesity and/or diabetes. Single or multiple administrations of the combinations and/or compositions may be administered depending on the dosage and frequency as needed and tolerated by the subject, e.g., patient. The particular dose used for a treatment will depend upon the medical condition and history of the subject, as well as other factors such as age, weight, gender, administration route, efficiency, etc.

In some embodiments, compounds used in the methods of the disclosure may be administered by any suitable route, including oral and intravenous routes. The compounds may be provided in pharmaceutical compositions comprising the compound and one or more pharmaceutically acceptable excipients. Formulation for oral administration may be in the form of a tablet or a capsule comprising a powder or liquid. Administration is preferably in a “therapeutically effective amount” or an “effective amount” (used interchangeably), this being sufficient to show benefit to the subject. The actual amount administered, and rate and time-course of administration, will depend on the nature and severity of the disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

In some embodiments, administration of a combination and/or a composition comprising (i) anti-ALK7 receptor antibody or antigen binding fragment thereof and (ii) second biological active agent provides increased weight loss in a subject comparable to the weight loss in a subject where an anti-ALK7 receptor antibody or antigen binding fragment thereof or an second biological active agent is administered alone.

In some embodiments, administration of a combination and/or a composition comprising (i) anti-ALK7 receptor antibody or antigen binding fragment thereof and (ii) second biological active agent results in increased fat mass loss while preserving lean mass (preventing lean mass loss) in a subject comparable to fat weight loss in in a subject where an anti-ALK7 receptor antibody or antigen binding fragment thereof is administered alone, or a second biological active agent is administered alone.

Provided herein are combinations, e.g., combination therapies, for use in prevention and/or treatment of overweight and/or obesity and methods for prevention and/or treatment of overweight and/or obesity. Generally, all subjects suffering from obesity are also includes those considered to be suffering from overweight. In some embodiments, those suffering from overweight does not include those suffering from obesity. In some embodiments, a subject suffering from overweight is human, such as an adult human or a paediatric human (including infants, children, and adolescents). In some embodiments a human subject suffering from overweight is classified by having a BMI of 25 kg/m² or more, such as a BMI of 27 kg/m² or more. In some embodiments, a human subject suffering from overweight has a BMI in the range of 25 to <30 or in the range of 27 to <30. In some embodiments, a subject suffering from obesity is human, such as an adult human or a paediatric human (including infants, children, and adolescents). Body mass index (BMI) is a measure of indicating an individual increased risk of having parameters associated this metabolic syndrome based on height and weight. The formula for calculation is BMI=weight in kilograms/height in meters². A human subject suffering from obesity may have a BMI of 30 kg/m² or more; this subject may also be referred to as person with obesity. In some embodiments, a human subject suffering from obesity may have a BMI of ≥35 or a BMI in the range of ≥30 to <40. In some embodiments, obesity is severe obesity or morbid obesity. In some embodiments, severe obesity or morbid obesity is when a human subject has a BMI of ≥40.

Provided herein are combinations for use in prevention and/or treatment of overweight, optionally in the presence of at least one weight-related comorbidity and methods for prevention and/or treatment of overweight, optionally in the presence of at least one weight-related comorbidity. A raised BMI increases the risk of a subject developing any one of a wide range of diseases or co-morbidities. In some embodiments, weight-related comorbidity is selected from the group consisting of insulin resistance, hypertension, diabetes (such as type 2 diabetes), dyslipidaemia, high cholesterol and obstructive sleep apnoea.

Provided herein are combinations for use in reducing body weight and methods for reducing body weight. In some embodiments, a human to be subjected to reduction of body weight has a BMI of 25 kg/m² or more, such as a BMI of 27 kg/m² or more (overweight) or a BMI of 30 kg/m² or more (obese). In some embodiments, a human to be subjected to reduction of body weight may have a BMI of 35 kg/m² or more or a BMI of 40 kg/m² or more. The term “reduction of body weight” may include treatment or prevention of obesity and/or overweight. In some embodiments, technologies (e.g., combinations, compositions, pharmaceutical compositions, methods and/or uses) according to the present disclosure is for use in reducing body fat mass. In some embodiments, technologies (e.g., combinations, compositions, pharmaceutical compositions, methods and/or uses) according to the present disclosure is for use in reducing body fat mass while maintaining lean body mass.

In some embodiments, compounds provided herein may be provided as a pharmaceutically acceptable salt, hydrate or solvate.

### Compositions

The present disclosure provides compositions comprising (i) an anti-ALK7 receptor antibody or antigen binding fragment thereof, and/or (ii) a second biological active agent as described herein. In some embodiments, a second biological active agent is or comprises a second biological active agent. In some embodiments, a composition comprises (i) an anti-ALK7 receptor antibody or antigen binding fragment thereof and (ii) a second biological active agent. In some embodiments, a composition comprises (i) an anti-ALK7 receptor antibody or antigen binding fragment thereof, and (ii) a second biological active agent comprising: (a) a GLP-1 receptor agonist, such as a GLP-1 receptor-amylin receptor agonist; (b) an anti-GIP receptor antibody or antigen binding fragment thereof, and/or (c) an anti-ActRII antibody or antigen binding fragment thereof.

In some embodiments, a composition as provided herein is for use as a medicament. In some embodiments, a composition as provided herein is for use in the treatment and/or prevention of overweight, obesity, and/or diabetes. In some embodiments, a composition as provided herein is for use in reducing body fast mass while maintaining lean body mass.

### Pharmaceutical Compositions

The present disclosure provides pharmaceutical compositions comprising an anti-ALK7 antibody or antigen binding fragment thereof as provided herein, a second biological active agent as provided herein, or a combination thereof.

In some embodiments, a pharmaceutical composition additionally comprises a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, a pharmaceutical composition comprising an anti-ALK7 antibody or antigen binding fragment thereof as provided herein, a second biological active agent as provided herein, or a combination thereof may be administered to a patient in need thereof at several sites, e.g. at topical sites such as skin or mucosal sites; at sites which bypass absorption such as in an artery, in a vein, or in the heart; and at sites which involve absorption, such as in the skin, under the skin, in a muscle, orally, or in the abdomen.

In some embodiments, a pharmaceutical composition as provided herein is for use as a medicament. In some embodiments, a pharmaceutical composition as provided herein is for use in the treatment and/or prevention of overweight, obesity, and/or diabetes. In some embodiments, a pharmaceutical composition as provided herein is for use in reducing body fast mass while maintaining lean body mass.

### Combinations

The present disclosure provides combinations, such as combination therapies. In some embodiments, combinations comprise (i) an anti-ALK7 receptor antibody or antigen binding fragment thereof, and (ii) a second biological active agent as provided herein. In some embodiments, a second biological active agent is or comprises a second biological active agent. In some embodiments, a combination comprises (i) an anti-ALK7 antibody or antigen binding fragment thereof and (ii) a second biological active agent. In some embodiments, a combination comprises (i) an anti-ALK7 receptor antibody or antigen binding fragment thereof, and (ii) a second biological active agent comprising: (a) a GLP-1 receptor agonist, such as a GLP-1 receptor-amylin receptor agonist; (b) an anti-GIP receptor antibody or antigen binding fragment thereof, and/or (c) an anti-ActRII antibody or antigen binding fragment thereof. In some embodiments, a combination is not formulated together. In some embodiments, a combination is formulated together. In some embodiments, a combination is not provided in the same composition. In some embodiments, a combination is provided in the same composition.

In some embodiments, the present disclosure provides a combination comprising: (i) an anti-ALK7 receptor antibody or antigen binding fragment thereof, and (ii) a second biological active agent comprising a GLP-1 receptor agonist.

In some embodiments, the present disclosure provides a combination comprising: (i) an anti-ALK7 receptor antibody or antigen binding fragment thereof, and (ii) a second biological active agent comprising a GLP-1 receptor-amylin receptor agonist.

In some embodiments, the present disclosure provides a combination comprising: (i) an anti-ALK7 receptor antibody or antigen binding fragment thereof, and (ii) a second biological active agent comprising an anti-GIP receptor antibody or antigen binding fragment thereof.

In some embodiments, the present disclosure provides a combination comprising: (i) an anti-ALK7 receptor antibody or antigen binding fragment thereof, and (ii) a second biological active agent comprising an anti-ActRII antibody or antigen binding fragment thereof.

In some embodiments, a combination is used in a combination therapy. In some embodiments, a combination as provided herein is for use as a medicament. In some embodiments, a combination as provided herein is for use in the treatment and/or prevention of overweight, obesity, and/or diabetes. In some embodiments, a combination as provided herein is for use in reducing body fast mass while maintaining lean body mass.

In some embodiments, the administration of two or more agents may be concomitantly or sequentially, i.e., not simultaneously. In some embodiments, the administration of two or more agents may be simultaneously, e.g., together in a single composition, such as a co-formulation. In some embodiments, combination therapy does not necessarily require that individual agents be administered together in a single composition (or even necessarily at the same time).

### Embodiments

1. An anti-ALK7 receptor antibody or antigen binding fragment thereof for use in prevention and/or treatment of overweight, obesity, and/or diabetes in combination with a second biological active agent.
2. The use of embodiment 1, wherein the anti-ALK7 receptor antibody or antigen binding fragment thereof is administered together with the second biological active agent.
3. The use of embodiment 1, wherein the anti-ALK7 receptor antibody or antigen binding fragment thereof is administered concomitantly or sequentially with the second biological active agent.
4. The use of any one of the preceding embodiments, wherein the second biological active agent comprises:
   (a) a GLP-1 receptor agonist;
   (b) an anti-GIP receptor antibody or antigen binding fragment thereof; and/or
   (c) anti-Act receptor II antibody or antigen binding fragment thereof.
5. The use of embodiment 4, wherein the GLP-1 receptor agonist comprises an amino acid sequence having at least 90% sequence identity to the amino acid sequence of SEQ ID NO: 2.
6. The use of embodiment 5, wherein the GLP-1 receptor agonist comprises an amino acid sequence of SEQ ID NO: 2.
7. The use of any one of embodiments 4-6, wherein the GLP-1 receptor agonist comprises the following structure (Formula I (SEQ ID NO: 106)):

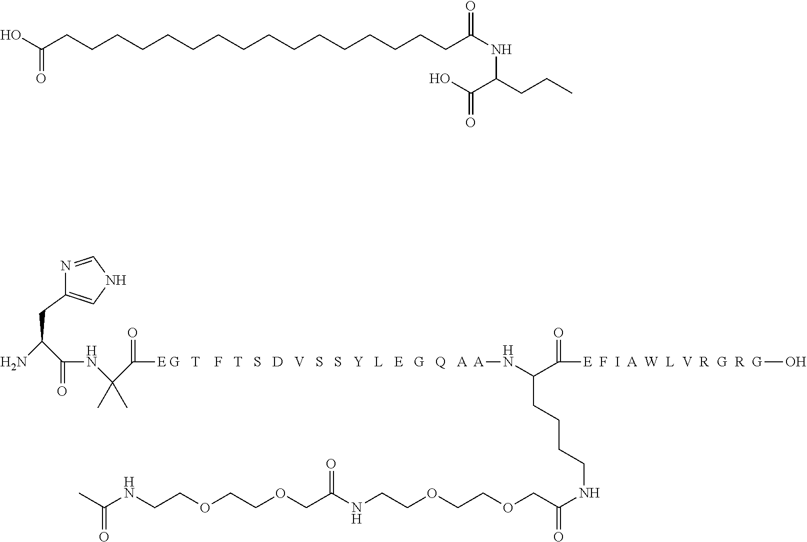

wherein the amino acid sequence is SEQ ID NO: 2, or a pharmaceutically acceptable salt thereof.
8. The use of any one of embodiments 4-6, wherein the GLP-1 receptor agonist comprises the following structure (Formula II (SEQ ID NO: 107)):

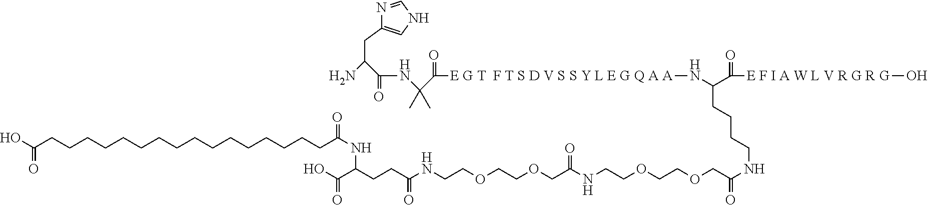

wherein the amino acid sequence is SEQ ID NO: 2, or a pharmaceutically acceptable salt thereof.
9. The use of any one of the preceding embodiments, wherein the second biological active agent comprises a GLP-1 receptor agonist and an amylin receptor agonist.
10. The use of any one of the preceding embodiments, wherein the second biological active agent comprises a GLP-1 receptor-amylin receptor agonist.
11. The use of embodiment 10, wherein the GLP-1 receptor-amylin receptor agonist comprises an amino acid sequence having at least 90% sequence identity to the amino acid sequence of SEQ ID NO: 3.
12. The use of embodiment 11, wherein the GLP-1 receptor-amylin receptor agonist comprises the following structure (Formula V (SEQ ID NO: 109)):

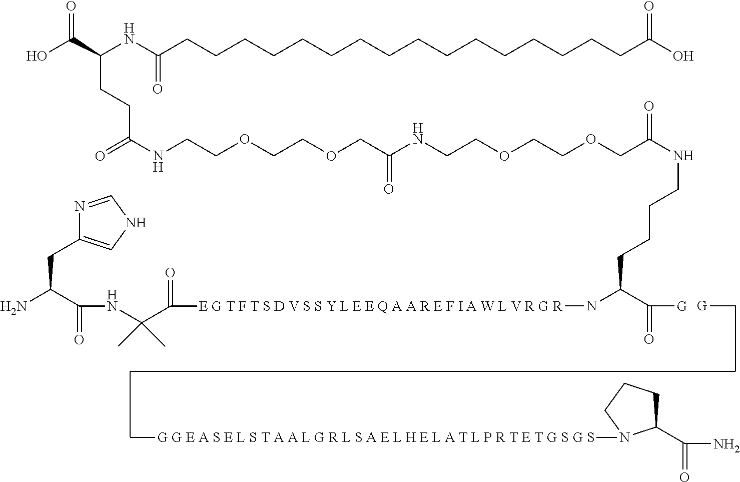

wherein the amino acid sequence is SEQ ID NO: 3, or a pharmaceutically acceptable salt thereof.
13. The use of embodiment 11, wherein the GLP-1 receptor-amylin receptor agonist comprises the following structure (Formula IV (SEQ ID NO: 108)):

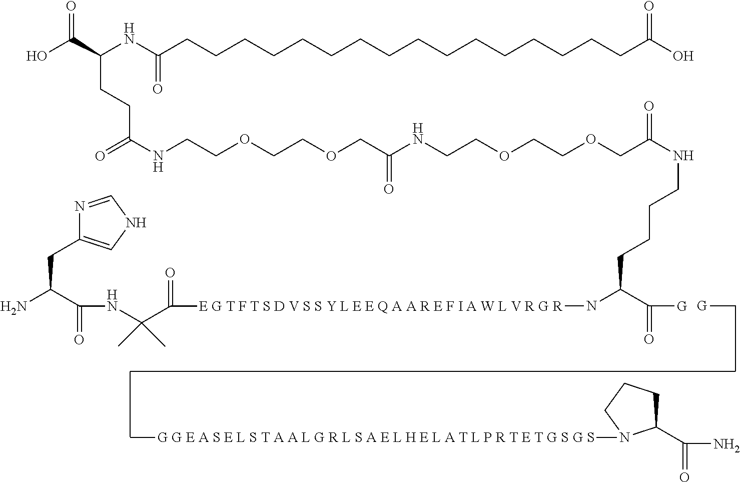

wherein the amino acid sequence is SEQ ID NO: 3, or a pharmaceutically acceptable salt thereof.
14. The use of any one of embodiments 4-13, wherein the anti-GIP receptor antibody or antigen binding fragment thereof comprises:
   (i) a heavy chain comprising an amino acid sequence having at least 90% sequence identity to the amino acid sequence of SEQ ID NO: 93; and/or
   (ii) a light chain comprising an amino acid sequence having at least 90% sequence identity to the amino acid sequence of SEQ ID NO: 94.
15. The use of any one of the preceding embodiments, wherein the anti-ALK7 receptor antibody or antigen binding fragment thereof is:
   (i) an antagonist;
   (ii) characterized in that it blocks binding of activin B to an ALK7 receptor; and/or
   (iii) characterized in that it blocks formation of a hexameric signaling complex of an ALK7 receptor with an ActRIIA/B Type II receptor.
16. The use of any one of the preceding embodiments, wherein the anti-ALK7 receptor antibody or antigen binding fragment thereof comprises three heavy chain CDR sequences, hCDR1-3, and three light chain CDR, sequences, ICDR1-3, wherein
   a) hCDR1 is SGNYWN (SEQ ID NO: 8) or SGSYWN (SEQ ID NO: 9) with up to 1 amino acid substitutions,
   b) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid substitutions,
   c) hCDR3 is DYYGSEGFGY (SEQ ID NO: 17) or DYYGSEGFAY (SEQ ID NO: 18) with up to 1 or 2 amino acid substitutions,
   d) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
   e) ICDR2 is WTSTRES (SEQ ID NO: 22) or WASTRES (SEQ ID NO: 23) with up to 1 amino acid substitutions, and
   f) ICDR3 is KQSYNLPYT (SEQ ID NO: 24) or KRSYNLPYT (SEQ ID NO: 25) with up to 1 amino acid substitutions.
17. The use of any one of the preceding embodiments, wherein the anti-ALK7 receptor antibody or antigen binding fragment comprises:
   i. a heavy chain variable region (HV) comprising an amino acid sequence according to any one of SEQ ID NO: 26-59, and/or
   ii. a light chain variable region (VL) comprising an amino acid sequence according to any one of SEQ ID NO: 60-84.
18. The use of any one of the preceding embodiments, wherein the anti-ALK7 receptor antibody or antigen binding fragment comprises:
   (i) a heavy chain comprising an amino acid sequence having at least 90% sequence identity to the amino acid sequence of SEQ ID NO: 99; and/or
   (ii) a light chain comprising an amino acid sequence having at least 90% sequence identity to the amino acid sequence of SEQ ID NO: 100.
19. The use of any one of the preceding embodiments, wherein the diabetes is type 1 diabetes or type 2 diabetes.
20. A method of treating a human subject being overweight, and/or having diabetes and/or obesity comprising:
   (i) administering an effective amount of anti-ALK7 receptor antibody or antigen binding fragment thereof, and
   (ii) administering an effective amount of second biological active agent.
21. The method of embodiment 20, wherein the anti-ALK7 receptor antibody or antigen binding fragment thereof is administered together with the second biological active agent.
22. The method of embodiment 20, wherein the anti-ALK7 receptor antibody or antigen binding fragment thereof is administered concomitantly or sequentially with the second biological active agent. 23. The method of any one of embodiments 20-22, wherein the second biological active agent comprises:
   (a) a GLP-1 receptor agonist;
   (b) an anti-GIP receptor antibody or antigen binding fragment thereof; and/or;
   (c) anti-Act receptor II antibody or antigen binding fragment thereof.
24. The method of embodiment 23, wherein the GLP-1 receptor agonist comprises an amino acid sequence having at least 90% sequence identity to the amino acid sequence of SEQ ID NO: 2.
25. The method of embodiment 24, wherein the GLP-1 receptor agonist comprises an amino acid sequence of SEQ ID NO: 2.
26. The method of embodiment 25, wherein the GLP-1 receptor agonist comprises the following structure (Formula I (SEQ ID NO: 106)):

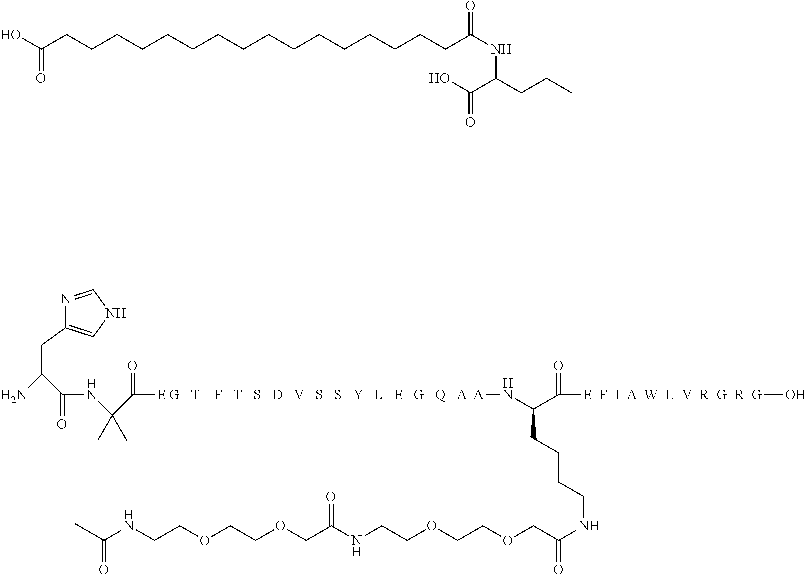

wherein the amino acid sequence is SEQ ID NO: 2, or a pharmaceutically acceptable salt thereof.
27. The method of embodiment 25, wherein the GLP-1 receptor agonist comprises the following structure (Formula II (SEQ ID NO: 107)):

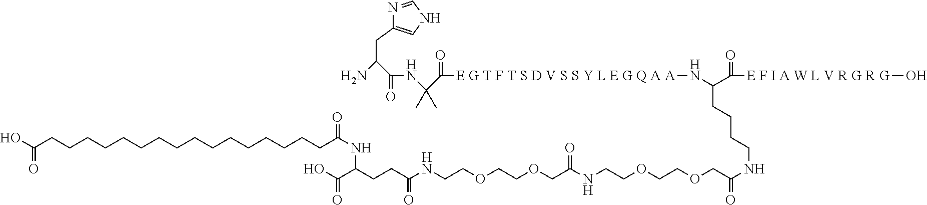

wherein the amino acid sequence is SEQ ID NO: 2, or a pharmaceutically acceptable salt thereof.
28. The method of any one of embodiments 20-27, wherein the second biological active agent comprises a GLP-1 receptor agonist and an amylin receptor agonist.
29. The method of any one of embodiments 20-27, wherein the second biological active agent comprises a GLP-1 receptor-amylin receptor agonist.
30. The method of embodiment 29, wherein the GLP-1 receptor-amylin receptor agonist comprises an amino acid sequence having at least 90% sequence identity to the amino acid sequence of SEQ ID NO: 3.
31. The method of embodiment 30, wherein the GLP-1 receptor-amylin receptor agonist comprises the following structure (Formula V (SEQ ID NO: 109)):

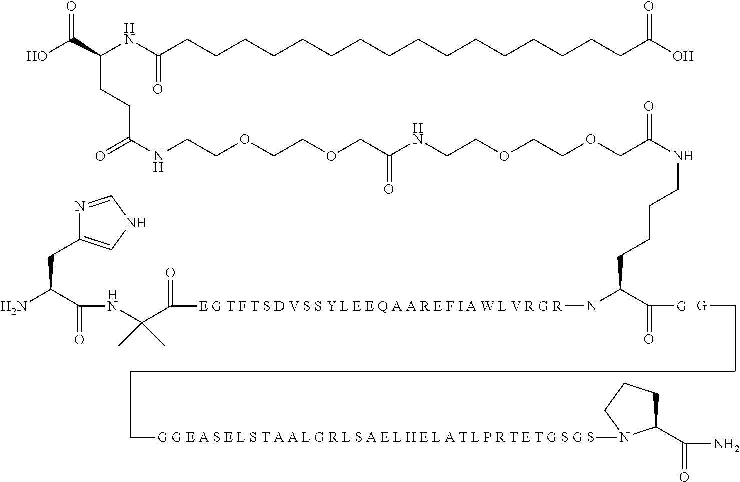

wherein the amino acid sequence is SEQ ID NO: 3, or a pharmaceutically acceptable salt thereof.
32. The method of embodiment 31, wherein the GLP-1 receptor-amylin receptor agonist comprises the following structure (Formula IV (SEQ ID NO: 108)):

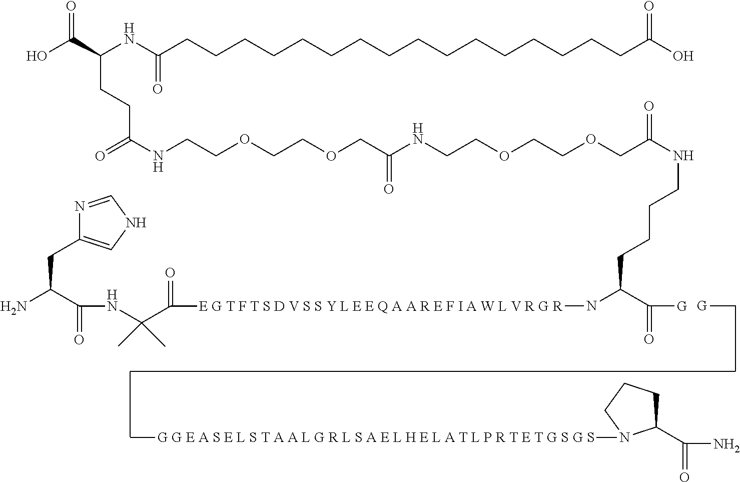

wherein the amino acid sequence is SEQ ID NO: 3, or a pharmaceutically acceptable salt thereof.
33. The method of any one of embodiments 24-32, wherein the anti-GIP receptor antibody or antigen binding fragment thereof comprises:
   (i) a heavy chain comprising an amino acid sequence having at least 90% sequence identity to the amino acid sequence of SEQ ID NO: 93; and/or
   (ii) a light chain comprising an amino acid sequence having at least 90% sequence identity to the amino acid sequence of SEQ ID NO 94.
34. The method of any one of embodiments 20-33, wherein the anti-ALK7 receptor antibody or antigen binding fragment thereof is:
   (i) an antagonist;
   (ii) characterized in that it blocks binding of activin B to an ALK7 receptor; and/or
   (iii) characterized in that it blocks formation of a hexameric signaling complex of an ALK7 receptor with an ActRIIA/B Type II receptor.
35. The method of any one of embodiments 20-34, wherein the anti-ALK7 receptor antibody or antigen binding fragment thereof comprises three heavy chain CDR sequences, hCDR1-3, and three light chain CDR, sequences, ICDR1-3, wherein
   a) hCDR1 is SGNYWN (SEQ ID NO: 8) or SGSYWN (SEQ ID NO: 9) with up to 1 amino acid substitutions,
   b) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid substitutions,
   c) hCDR3 is DYYGSEGFGY (SEQ ID NO: 17) or DYYGSEGFAY (SEQ ID NO: 18) with up to 1 or 2 amino acid substitutions,
   d) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
   e) ICDR2 is WTSTRES (SEQ ID NO: 22) or WASTRES (SEQ ID NO: 23) with up to 1 amino acid substitutions, and
   f) ICDR3 is KQSYNLPYT (SEQ ID NO: 24) or KRSYNLPYT (SEQ ID NO: 25) with up to 1 amino acid substitutions.
36. The method of any one of embodiments 20-35, wherein the anti-ALK7 receptor antibody or antigen binding fragment comprises:
   i. a heavy chain variable region (VH) comprising an amino acid sequence according to any one of SEQ ID NO: 26-59, and/or
   ii. a light chain variable region (VL) comprising an amino acid sequence according to any one of SEQ ID NO: 60-84.
37. The method of any one of embodiments 20-36, wherein the anti-ALK7 receptor antibody or antigen binding fragment comprises:
   (i) a heavy chain comprising an amino acid sequence having at least 90% sequence identity to the amino acid sequence of SEQ ID NO: 99; and/or
   (ii) a light chain comprising an amino acid sequence having at least 90% sequence identity to the amino acid sequence of SEQ ID NO 100.
38. The method of any one of embodiments 20-37, wherein the diabetes is type 1 diabetes or type 2 diabetes.
39. A combination comprising:
   (i) an anti-ALK7 receptor antibody or antigen binding fragment thereof, and
   (ii) a second biological active agent comprising:
      (a) a GLP-1 receptor agonist;
      (b) an anti-GIP receptor antibody or antigen binding fragment thereof; and/or
      (c) anti-Act receptor II antibody or antigen binding fragment thereof.
40. The combination of embodiments 39, wherein the GLP-1 receptor agonist comprises an amino acid sequence having at least 90% sequence identity to the amino acid sequence of SEQ ID NO: 2.
41. The combination of embodiments 39-40, wherein the GLP-1 receptor agonist comprises an amino acid sequence of SEQ ID NO: 2.
42. The combination of any one of embodiments 39-41, wherein the GLP-1 receptor agonist comprises the following structure (Formula I (SEQ ID NO: 106))

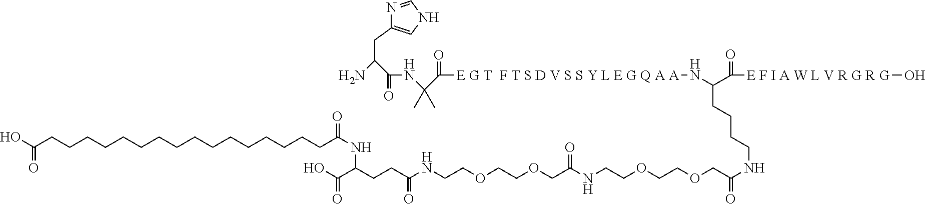

wherein the amino acid sequence is SEQ ID NO: 2, or a pharmaceutically acceptable salt thereof.
43. The combination of any one of embodiments 39-41, wherein the GLP-1 receptor agonist comprises the following structure (Formula II (SEQ ID NO: 107))

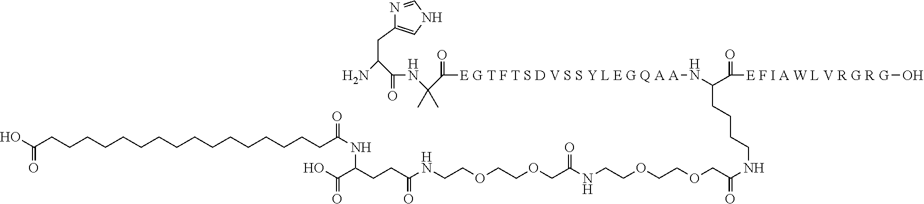

wherein the amino acid sequence is SEQ ID NO: 2, or a pharmaceutically acceptable salt thereof.
44. The combination of any one of embodiments 39-43, wherein the second biological active agent comprises a GLP-1 receptor agonist and an amylin receptor agonist.
45. The combination of any one of embodiments 39-44, wherein the second biological active agent comprises a GLP-1 receptor-amylin receptor agonist.
46. The combination of embodiment 45, wherein the GLP-1 receptor-amylin receptor agonist comprises an amino acid sequence having at least 90% sequence identity to the amino acid sequence of SEQ ID NO: 3.
47. The combination of embodiment 46, wherein the GLP-1 receptor-amylin receptor agonist comprises the following structure (Formula IV (SEQ ID NO: 108)):

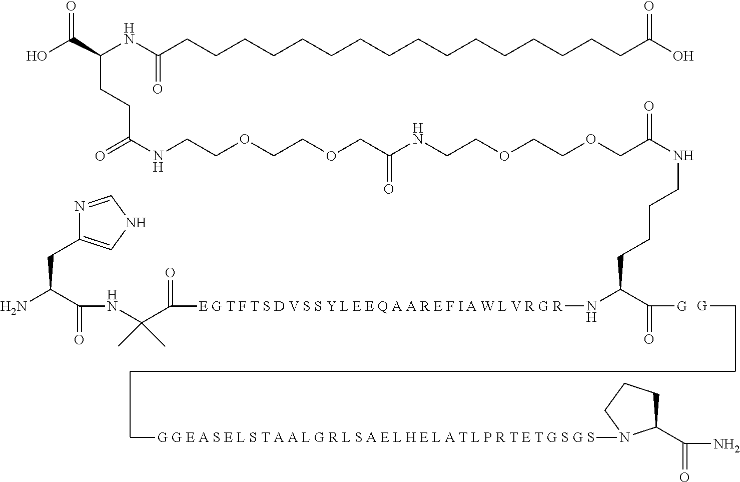

wherein the amino acid sequence is SEQ ID NO: 3, or a pharmaceutically acceptable salt thereof.
48. The combination of embodiment 46, wherein the −GLP-1 receptor-amylin receptor agonist comprises the following structure (Formula V (SEQ ID NO: 109)):

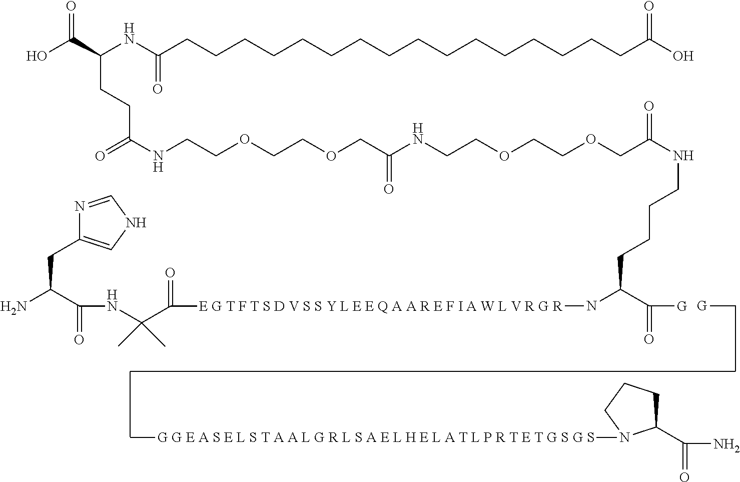

wherein the amino acid sequence is SEQ ID NO: 3, or a pharmaceutically acceptable salt thereof.
49. The combination of any one of embodiments 39-48, wherein the anti-GIP receptor antibody or antigen binding fragment thereof comprises:
   (i) a heavy chain comprising an amino acid sequence having at least 90% sequence identity to the amino acid sequence of SEQ ID NO: 93; and/or
   (ii) a light chain comprising an amino acid sequence having at least 90% sequence identity to the amino acid sequence of SEQ ID NO 94.
50. The combination of any one of embodiments 39-49, wherein the anti-ALK7 receptor antibody or antigen binding fragment thereof is:
   (i) an antagonist;
   (ii) characterized in that it blocks binding of activin B to an ALK7 receptor; and/or (iii) characterized in that it blocks formation of a hexameric signaling complex of an ALK7 receptor with an ActRIIA/B Type II receptor.
51. The combination of any one of embodiments 39-50, wherein the anti-ALK7 receptor antibody or antigen binding fragment thereof comprises three heavy chain CDR sequences, hCDR1-3, and three light chain CDR, sequences, ICDR1-3, wherein
   a) hCDR1 is SGNYWN (SEQ ID NO: 8) or SGSYWN (SEQ ID NO: 9) with up to 1 amino acid substitutions,
   b) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid substitutions,
   c) hCDR3 is DYYGSEGFGY (SEQ ID NO: 17) or DYYGSEGFAY (SEQ ID NO: 18) with up to 1 or 2 amino acid substitutions,
   d) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
   e) ICDR2 is WTSTRES (SEQ ID NO: 22) or WASTRES (SEQ ID NO: 23) with up to 1 amino acid substitutions and
   f) ICDR3 is KQSYNLPYT (SEQ ID NO: 24) or KRSYNLPYT (SEQ ID NO: 25) with up to 1 amino acid substitutions.
52. The combination of any one of embodiments 39-51, wherein the anti-ALK7 receptor antibody or antigen binding fragment comprises:
   i. a heavy chain variable region (VH) comprising an amino acid sequence according to any one of SEQ ID NO: 26-59, and/or
   ii. a light chain variable regions (VL) comprising an amino acid sequence according to any one of SEQ ID NO: 60-84.
53. The combination of any one of embodiments 39-52, wherein the anti-ALK7 receptor antibody or antigen binding fragment comprises:
   (i) a heavy chain comprising an amino acid sequence having at least 90% sequence identity to the amino acid sequence of SEQ ID NO: 99; and/or
   (ii) a light chain comprising an amino acid sequence having at least 90% sequence identity to the amino acid sequence of SEQ ID NO 100.
54. A combination comprising:
   (i) an anti-ALK7 receptor antibody or antigen binding fragment thereof, and
   (ii) a GLP-1 receptor agonist.
55. A combination comprising:
   (i) an anti-ALK7 receptor antibody or antigen binding fragment thereof, and
   (ii) a GLP-1 receptor-amylin receptor agonist.
56. A combination comprising:
   (i) an anti-ALK7 receptor antibody or antigen binding fragment thereof, and
   (ii) an anti-GIP receptor antibody or antigen binding fragment thereof.
57. A combination comprising:
   (i) an anti-ALK7 receptor antibody or antigen binding fragment thereof, and
   (ii) an anti-ActRII antibody or antigen binding fragment thereof.
58. A pharmaceutical combination comprising the combination of any one of embodiments 39-57 and at least one pharmaceutically acceptable excipient.
59. The combination of any one of embodiments 39-57 and the pharmaceutical combination of embodiment 58 for use as a medicament.
60. The combination of any one of embodiments 39-57 and the pharmaceutical combination of embodiment 58 for use in the prevention or treatment of obesity.
61. The use of any one of embodiments 1-19, the method of any one of embodiments 20-38, the combination of any one of embodiments 39-57 and the pharmaceutical combination of embodiment 58 for use in reducing body fat mass.
62. The use of any one of embodiments 1-19, the method of any one of embodiments 20-38, the combination of any one of embodiments 39-57 and the pharmaceutical combination of embodiment 58 for use in maintaining lean body mass.
63. Use of a combination of any one of embodiments 39-57 and the pharmaceutical combination of embodiment 58 in medicine.
64. Use of any one of embodiments 1-19 and 63 and the pharmaceutical combination of embodiment 58 for prevention or treatment of obesity.
65. Use of any one of embodiments 1-19 and 63 and the pharmaceutical combination of embodiment 58 for reducing body fat mass.
66. The use according to embodiment 65, wherein the lean body mass is preserved.

### EXAMPLES

List of Abbreviations

| | |
|---|---|
| Boc: | t-butyloxycarbonyl |
| CAD: | Charged Aerosol Detector |
| CLND: | Chemiluminescent Nitrogen Detection |
| DCM: | Dichloromethane |
| DIC: | Diisopropylcarbodiimide |
| DMF: | Dimethylformamide |
| DMSO: | Dimethylsulfoxide |
| DTT: | Dithiothreitol |
| Et2O: | Diethyl ether |
| Fmoc: | 9-fluorenylmethyloxycarbonyl |
| HPLC: | High Performance Liquid Chromatography |
| LC: | Liquid Chromatography |
| LCMS: | Liquid Chromatography Mass Spectroscopy |
| MALDI-MS: | Matrix-Assisted Laser Desorption Ionization |
| | Mass Spectrometry |
| MeCN | Acetonitrile |
| MQ: | Milli-Q |
| MS: | Mass Spectroscopy |
| NMP: | 1-Methyl-pyrrolidin-2-one |
| ADO | 8-amino-3,6-dioxaoctanoic acid |
| OtBu: | tert-butoxy |
| Oxyma Pure ®: | Cyano-hydroxyimino-acetic acid ethyl ester |
| Pbf: | 2,2,4,6,7-pentamethyldihydrobenzofuran-5- |
| | sulfonyl |
| RP: | Reverse Phase |
| RP-HPLC: | Reverse Phase High Performance Liquid |
| | Chromatography |
| RT: | Room Temperature |
| SEC: | Size-Exclusion Chromatography |
| SPPS: | Solid Phase Peptide Synthesis |
| tBu: | tert-butyl |
| TFA: | trifluoroacetic acid |
| TIPS: | triisopropylsilane |
| Trt: | triphenylmethyl (trityl) |
| UPLC: | Ultra Performance Liquid Chromatography |
| UV: | Ultraviolet |

### Example 1: Methods for Preparing Peptides

This section provides exemplary methods for solid phase peptide synthesis (SPPS), including methods for de-protection of amino acids, methods for cleaving a peptide from a resin, and for its purification, as well as methods for detecting and characterising the resulting peptide (LCMS methods).

### Fatty Acid Building Blocks

For synthesis of octadecanedioic acid mono-tert-butyl ester, see WO2010/102886, pages 27-28, herein incorporated by reference. The corresponding mono-tert-butyl esters of C20 diacid can be prepared accordingly.

### General Method for Peptide Synthesis

C-terminal peptide amides were prepared using an amine-based resin (e.g. H-Rink Amide-MBHA resin, with loading of e.g. 0.5 mmol/g). Peptides with C-terminal carboxylic acid were prepared using a hydroxyl-derived resin (e.g. Wang resin), optionally preloaded with the first amino acid (e.g. Fmoc-Gly-Wang resin with loading of 0.4 mmol/g). The Fmoc-protected amino acid derivatives used, unless specifically stated otherwise, were the standard recommended: Fmoc-Ala-OH, Fmoc-Arg (Pbf)-OH, Fmoc-Asn (Trt)-OH, Fmoc-Asp (OtBu)-OH, Fmoc-Cys (Trt)-OH, Fmoc-Gln (Trt)-OH, Fmoc-Glu (OtBu)-OH, Fmoc-Gly-OH, Fmoc-His (Trt)-OH, Fmoc-IIe-OH, Fmoc-Leu-OH, Fmoc-Lys (Boc)-OH, Boc-Lys (Fmoc)-OH, Fmoc-Met-OH, Fmoc-Phe-OH, Fmoc-Pro-OH, Fmoc-Ser (tBu)-OH, Fmoc-Thr (tBu)-OH, Fmoc-Trp (Boc)-OH, Fmoc-Tyr (tBu)-OH, Fmoc-Val-OH, Fmoc-D-Tyr-(tBu)-OH, etc. supplied from e.g. ChemImpex, Combi-Blocks, Gyros Protein Technologies, Iris Biotech, or Novabiochem. Where nothing else is specified, the natural L-form of the amino acids are used.

In case of modular albumin binding moiety attachment using SPPS, the following suitably protected building blocks such as but not limited to Boc-Lys (Fmoc)-OH, Fmoc-8-amino-3,6-dioxaoctanoic acid (Fmoc-Ado-OH), Fmoc-Glu-OtBu, octadecanedioic acid mono-tert-butyl ester, and eicosanedioic acid mono-tert-butyl ester were used. All operations stated below were performed within a 0.1-0.2 mmol synthesis scale range.

Unless otherwise specified, SPPS was typically performed using Fmoc based chemistry on a Gyros Protein Technologies SymphonyX solid phase peptide synthesizer, using the manufacturer supplied protocols with minor modifications. Mixing was accomplished by occasional bubbling with nitrogen. The step-wise assembly was performed using the following steps: 1) pre-swelling of resin in DMF; 2) Fmoc-deprotection by the use of 20% (v/v) piperidine in DMF for two treatments of 10 min each; 3) washes with DMF to remove piperidine; 4) coupling of Fmoc-amino acid by the addition of Fmoc-amino acid (5 equiv) and Oxyma Pure® (5 equiv) as a 0.5 M solution each in DMF, followed by addition of collidine (10 equiv) as a 1 M solution in DMF, followed by addition of DIC (10 equiv) as a 0.5 M solution in DMF, then mixing for 0.5-4 h; 5) draining liquid reagents followed by capping of unreacted peptidyl resin by addition of acetic anhydride (10 equiv) as a 1 M solution in DMF followed by addition of DMF to reach a final acetic anhydride concentration of 0.25 M; 6) washes with DMF to remove excess reagents; 7) final sequential washes with DCM, MeOH, and Et₂O at the completion of the assembly. Some amino acids such as (but not limited to) Arg or those following a sterically hindered amino acid such as Thr were coupled with an extended reaction time (e.g. 4 h) to ensure reaction completion. Boc-Lys (Fmoc)-OH, Fmoc-Ado-OH, Fmoc-Glu-OtBu, octadecanedioic acid mono-tert-butyl ester, and eicosanedioic acid mono-tert-butyl ester were coupled using the above procedures but with 10 equiv of each amino acid or carboxylic acid-containing building block as well as 10 equiv of Oxyma Pure®, and coupling times were extended to 4 h. Reagents such as eicosanedioic acid mono-tert-butyl ester were prepared as a 0.25 M solution in DMF to accommodate the lower solubility in DMF.

### General Cleavage Method

The peptides were cleaved from the polystyrene resin with TFA/TIPS/H₂O/Thioanisole/DTT (90:2.5:2.5:2.5:2.5 vol %) for 2 hours, after which the solution was drained into cold diethyl ether and centrifuged. The ether was decanted off, and the peptide was washed thusly with diethyl ether two more times.

### General Method for Purification and Quantification of the Derivative

The crude peptide was dissolved in water with occasional addition of minimal amounts of MeCN and/or DMSO and/or TFA to facilitate dissolution in a minimum volume, then this solution was filtered through e.g. a 0.2 μm filter. The crude peptides were then purified by reversed-phase preparative HPLC (e.g. Waters Prep 150 LC) on a column comprising C18-silica gel. Elution was performed with an increasing gradient of MeCN in MQ water containing 0.1% TFA. Relevant fractions were analysed with MALDI-MS and UPLC. Fractions containing the pure target peptide were pooled. The resulting solution was analysed (UPLC, LCMS) and the peptide derivative was quantified using a CLND specific HPLC detector (Ultimate-3000 Thermo-Fischer HPLC connected to an Antek 8060 CLND). The product was dispensed into glass vials. The vials were capped with Millipore glass fibre prefilters. Freeze-drying afforded the trifluoroacetate salt of the derivative as a white solid.

Exemplary peptides, used in the following examples, are shown in Table 1.

TABLE 1
Biological activity and modality type.

| Primary Activity | Modality | SEQ ID NO | Production |
|---|---|---|---|
| GLP-1 receptor agonist A | Fatty acid protracted peptide | 2 (Formula I) | SPPS |
| GLP-1 receptor agonist B | Fatty acid protracted peptide | 2 (Formula II) | SPPS |
| GLP-1 receptor-amylin | Fatty acid protracted peptide | 3 (Formula IV) | SPPS |
| receptor agonist A | | | |
| GLP-1 receptor-amylin | Fatty acid protracted peptide | 3 (Formula V) | SPPS |
| receptor agonist B | | | |

### Example 2: Methods for Preparing Antibodies

For each monoclonal antibody heavy and light chain, DNA encoding the human CD33 signal peptide followed in frame by the mature antibody chain was synthesized and inserted between the Hind III and Eco RI sites of the mammalian expression vector pTT5 (Durocher Y, Perret S, Kamen A. (2002) High-level and high-throughput recombinant protein production by transient transfection of suspension-growing human 293-EBNA1 cells. Nucleic Acids Res 30: E9). In the resulting plasmids, the antibody chains are under the transcriptional control of the cytomegalo virus promoter.

1000-mL cultures of suspension adapted human embryonal kidney (Expi293F) cells (ThermoFisher Scientific) in Expi293 Expression Medium (ThermoFisher Scientific) were grown to a density of approximately 3×10⁶ cells/mL in 3000-mL Erlenmeyer shaker flasks. For each 1000-mL culture, 500 μg antibody heavy chain coding plasmid and 500 μg antibody light chain coding plasmid was diluted in 50 mL Opti-MEM I Reduced Serum Medium (ThermoFisher Scientific). ExpiFectamine 293 Reagent (ThermoFisher Scientific) was diluted to 5.1% in Opti-MEM I Reduced Serum Medium, incubated for 5 minutes at room temperature, and then added to the diluted plasmids in portions of 52.7 mL. The plasmid DNA complexation reactions were gently mixed, incubated for 10-20 minutes at room temperature, and then gently transferred to the Expi293F cultures. Subsequently, the Expi293F cultures were incubated in a CO₂ shaker (85 rpm orbital shaking, 36.5° C., and 8% CO₂). One day after transfection, each culture flask was added 5 mL ExpiFectamine 293 Transfection Enhancer 1 (ThermoFisher Scientific) and 50 mL ExpiFectamine 293 Transfection Enhancer 2 (ThermoFisher Scientific). Five days after transfection, the cells were pelleted by centrifugation and discarded. The supernatants were further cleared by filtration through a 0.22 μm polyethersulfone filter using a Corning filter system (Corning Inc.) and utilized for purification of monoclonal antibodies.

Monoclonal antibodies were purified by affinity chromatography on MabSelect Sure followed by size exclusion chromatography (SEC) on Superdex 200. Briefly, 0.5-2 L harvest was loaded on 5 mL HiTrap MabSelect SuRe (Cytiva cat.no. 11.0034-95) equilibrated with 20 mM HEPES, 150 mM NaCl pH 7.4 at a flow rate of 16 column volumes (CVs) per h. Following the wash step with 8 CVs of the equilibration buffer, antibodies were eluted with 50 mM formic acid, 150 mM NaCl pH 3.5. Eluted proteins (elution volume 15 mL) were purified directly without holding time on Superdex 200 26/600 column, 320 ml (Cytiva cat.no. 28-9893-36). SEC was performed at a flow rate of 0.5 CV/h using 20 mM HEPES, 150 mM NaCl pH 7.4 as a running buffer. Fractions containing monomeric mAb were pooled and analysed by SE-UPLC and LC-MS. Exemplary antibodies are provided in Table 2.

TABLE 2
Biological activity and modality type.

| Primary Activity | Modality | SEQ ID NO | Production |
|---|---|---|---|
| Anti-GIP receptor | Antagonist | 4 | Mammalian |
| antibody | | 93 (HC) | cell process |
| | | 94 (LC) | |
| Anti-Activin receptor | Antagonist | 5 | Mammalian |
| type II antibody | | 95 (HC) | cell process |
| | | 96 (LC) | |
| Anti-ALK7 receptor | Antagonist | 6 | Mammalian |
| antibody A | | 97 (HC) | cell process |
| | | 98 (LC) | |
| Anti-ALK7 receptor | Antagonist | 99 (HC) | Mammalian |
| antibody B | | 100 (LC) | cell process |

### Example 3: In Vitro Potency of an Anti-ALK7 Receptor Blocking Antibody

### Receptor Potency Using RGA Assay

A reporter gene assay (RGA) was established to determine the ability of anti-ALK7 receptor blocking antibodies to inhibit ALK7 signaling. The luciferase gene was used, under control of Smad binding element (SBE) to measure ALK7 signaling, since stimulated ALK7 is known to phosphorylate Smad3, which binds SBE. Thus, stimulation of ALK7 in cells containing SBE-luciferase results in increased luciferase expression.

HEK293 cells (ATCC CRL-1573) were stably transfected with the SBE-luciferase reporter plasmid pGL4.48 [luc2P/SBE/Hygro] from Promega and a single cell clone was isolated. This single cell clone was stably transfected with a human ALK7-S270T expression plasmid based on pcDNA3.1(+) and a single cell clone, named clone 6, was isolated. The S270T mutation renders ALK7 insensitive to the SB431542 inhibitor, which was subsequently used to avoid stimulation of endogenous ALK receptors.

For measuring inhibition of ALK7 signaling, the HEK293/hALK7-S270T/SBE-luciferase (clone 6) cells were seeded in white, opaque 384-well plates at a density of 20000 cells/well. The following day medium was removed from the assay plates and compounds diluted in medium containing 1.36 μM SB431542 was added. After 30 min incubation at 37° C., 0.5 nM human Activin B (R&D Systems) diluted in medium containing 1.36 UM SB431542 was added followed by a 4 h incubation at 37° C. Cells were lysed and luciferase substrate added using the Steady-GLO kit (Promega). Luminescence was detected using the EnVision 2104 plate reader with ultrasensitive luminescence detection (PerkinElmer). DMEM supplemented with 10% fetal bovine serum and 1% penicillin/streptomycin was used for seeding cells and dilution of compounds and agonist. For determining IC₅₀ values, antibody concentration-response curves were fitted to the four-parameter logistic equation.

### Primary Human Subcutaneous Preadipocytes

Primary Human Subcutaneous Preadipocytes were isolated from human subcutaneous adipose tissue by enzymatic digestion and selective culturing techniques and purchased frozen as unpassaged preadipocytes in single use cryotubes. Once thawed, cells were suspended in Preadipocyte Growth Medium-2 supplemented with Fetal bovine serum, L-glutamine, and GA-1000 SingleQuots™, and 10000 cells/well were dispensed in 96 well plates and incubated at 37° C., 5% CO₂ and 90% humidity for 24 hours. The subsequent day, the preadipocytes were differentiated by adding Growth Medium-2 containing above supplements with addition of insulin, dexamethasone, indomethacin and isobutyl-methylxanthine and differentiated for 10 days. After differentiation cells are starved for 24 hours in subcutaneous basal medium containing bovine serum albumin and assayed the next day.

p-SMAD3 Assay

To detect phospho-SMAD3 (phosphorylated on Ser423/Ser425) in cellular lysates, the AlphaLISA® SureFire® Ultra™ p-SMAD3 (Ser423/Ser425) assay which is a sandwich immunoassay was used, using Alpha Technology. Starving media was removed from the cells and recombinant human Activin B (an ALK7 receptor ligand), at a constant concentration, and ALK7 blocking antibodies, serial diluted, in subcutaneous basal medium containing tween were added to the cells and incubated for 40 min. Media was then removed and lysis buffer was added to the cells. Plates were flash frozen to ensure proper cell lysis. 10 μl of cell lysate was transferred to untreated AlphaPlate-384 and 5 μl of acceptor bead mix was added to wells and incubated for 1 hour followed by addition of 5 μl of donor bead mix and incubated over night at room temperature protected from light. Plates were read on an EnVision™ plate reader (excitation 680 nm, emission 615 nm) and IC₅₀ values were calculated in Graph Pad Prism by non-linear regression curve fitting. Anti-ALK7 receptor antibody (SEQ ID NO: 6, comprising SEQ ID NO: 97 and SEQ ID NO: 98) IC₅₀ value is shown in Table 3 herein.

TABLE 3
In vitro potency of an exemplary anti-ALK7 antibody.

| | | | Primary Human | |
|---|---|---|---|---|
| | Receptor potency | | Subcutaneous | |
| | RGA assay | | Preadipocytes | |
| | Inhibition of | | Inhibition of | |
| | Activin B induced | | Activin B induced | |
| | p-SMAD-3 | | p-SMAD-3 | |
| | | Numbers of | | Number of |
| | IC₅₀ | experiments | IC₅₀ | experimetns |
| | nanoMolar | (n) | nanoMolar | (n) |
| Anti-ALK7 | 1.7 | 90 | 1.9 | 36 |
| receptor | | | | |
| antibody A | | | | |

### Example 4: In Vitro Potency of an Anti-GIP Receptor Blocking Antibody

### Receptor Potency Using RGA Assay

A reporter gene assay (RGA) was used to determine the ability of the anti-GIPR antibodies to inhibit GIP signalling. The assay uses luciferase under control of CAMP response element (CRE) to measure mouse GIPR signalling, since GIPR is known to signal through the heterotrimeric G protein Gs, resulting in stimulation of cAMP production by adenylyl cyclase. Stimulation of mouse GIPR in cells containing CRE-luciferase results in increased luciferase expression.

BHK-21 cells (ATCC CCL-10) were stably transfected with a mouse GIPR expression plasmid and a CRE-luciferase reporter plasmid. The mouse GIPR expression plasmid is based on pcDNA3.1(+). The reporter plasmid is pGL4.29 [luc2P/CRE/Hygro] from Promega.

For measuring inhibition of mouse GIPR signalling, the BHK21/mGIPR/CRE-luciferase cells were seeded in white, opaque 384-well plates at a density of 5000 cells/well. The following day the medium was removed from the assay plates and antibodies diluted to the desired concentrations were added. After 30 min incubation at 37° C., mGIP was added followed by 4 h incubation at 37° C. Cells were lysed and luciferase substrate added using the Steady-GLO kit (Promega). Luminescence was detected using the EnVision 2104 plate reader with ultrasensitive luminescence detection (PerkinElmer).

For determining IC₅₀ values for antibodies, a range of antibody concentrations and a single concentration of mGIP approximately corresponding to its EC₈₀ (25 pM) were added to the cells. The antibody concentration-response curves were normalized to the minimum response in presence of a competitive antibody control (0%) and the response in absence of antibody (100%) and fitted to the four-parameter logistic equation. Anti-GIP receptor antibody IC₅₀ value is shown in Table 4 herein.

TABLE 4
In vitro potency of an exemplary anti-GIPR antibody

| | Mouse GIPR antagonist assay |
|---|---|
| | IC₅₀ nanoMolar |
| GIPR receptor blocking mAb | 4.352 |

### Example 5: Administration of Anti-ALK7 Receptor Antibody a and GLP-1 Receptor Agonist in Mice

The purpose of this Example is to determine the effect of a s.c. injection of an exemplary anti-ALK7 receptor antibody in diet induced obese (DIO) mice on total body weight, fat mass, and lean mass with and without co-administration with a GLP-1 receptor agonist.

### Study

A four-week study in DIO mice (n=8) investigating the body weight lowering effect of anti-ALK7 inhibiting antibody (ALK7i Ab), GLP-1 receptor agonist (GLP-1RA), alone or in combination. Male, C57BI/6J mice began high fat diet (HFD) feeding at 10 weeks-old (45% HFD; Research Diets) and throughout the study; study start at 53 weeks of age. Mice were single housed and food intake was measured daily. Male, DIO mice were dosed with an anti-ALK7 antibody (10 mg/kg, s.c., twice weekly), an GLP-1 receptor agonist (1 nmol/kg, s.c. daily), or a both (Combo group). Control mice were dosed with both an isotype control antibody (10 mg/kg in PBS, s.c., twice weekly) and Vehicle (s.c. daily). Body composition was measured at the end of the study via EchoMRI. 22-24 hours prior to sacrifice, mice were given 1.5 g of high fat diet (~6 hour before the dark cycle) at the time of last dose resulting in a “semi-fast”. Immediately prior to sacrifice mice, blood glucose was measured from the tail. Mice were then sacrificed 22-24 h following the last dose. Following sacrifice plasma was collected for insulin measurements via ELISA and the liver was dissected out and fat and lean mass content was determined via EchoMRI. The study design was as shown in Table 5.

TABLE 5
Study design.

| | | | Dose | |
|---|---|---|---|---|
| | | | (mg/kg; | Dose |
| | | | twice | (nmol/kg; |
| Group | Tx | Route | weekly) | QD) |
| 1 | Controls (isotype) | SC | 10 | — |
| 2 | Anti-ALK7 antibody A | SC | 10 | — |
| | (ALK7i Ab) | | | |
| 3 | GLP-1 receptor agonist A | SC | — | 1 |
| | (GLP-1RA of Formula I) | | | |
| 4 | Anti-ALK7 antibody A + | SC | 10 | 1 |
| | GLP-1RA of Formula I | | | |
| | (Combo) | | | |

QD: Daily Dosing

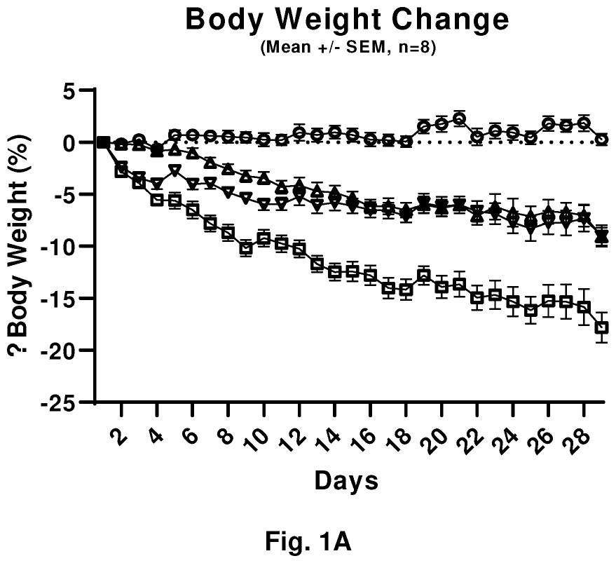

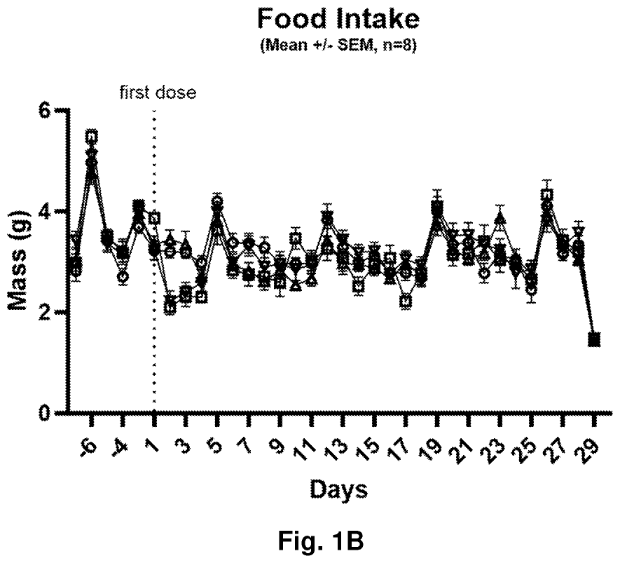

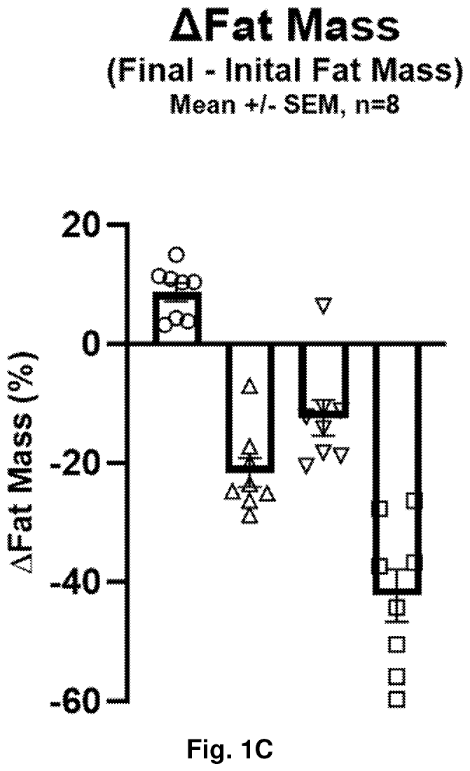

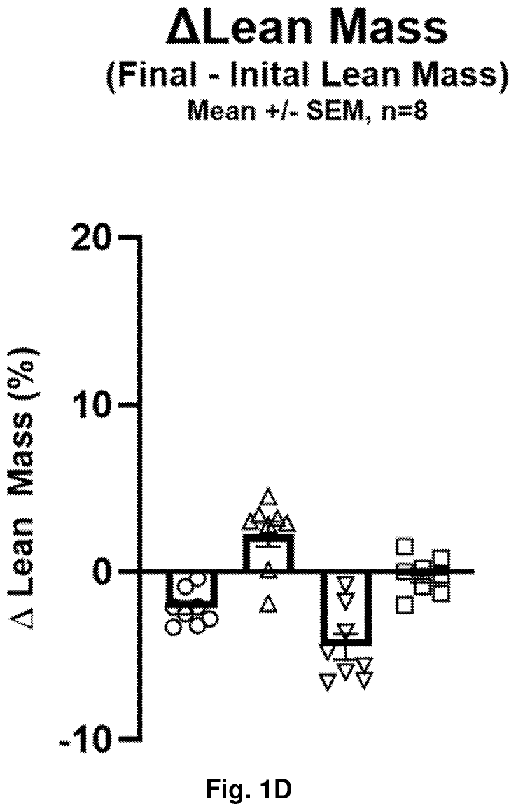

The control group maintained their body weight during the study. The groups receiving either anti-ALK7 inhibiting antibody (ALK7i Ab) or GLP-1 receptor agonist (GLP-1 RA) alone showed a comparable weight loss of approximately 9% at day 29, whereas the Combo group receiving both ALK7i Ab and GLP-1 RA showed a synergistic weight loss of approximately 18% at day 29 (FIG. 1A).

Weight loss with ALK7i Ab mono treatment alone was not associated with reduced food intake. GLP-1RA mono treatment induced weight loss was associated with a transient reduction in food intake. Despite increased weight loss in the Combo group versus the mono treatment groups, this was not associated with further reductions in food intake compared with GLP-1RA treatment alone (FIG. 1B). Mice in the control group gained fat mass, whereas they lost lean mass. ALK7i Ab induced total weight loss was due to fat mass loss with no loss in lean mass; i.e., prevention of loss of lean mass during wight loss. GLP-1RA induced total weight loss was due to both fat mass and lean mass loss. Combination treatment of ALK7i Ab and GLP-1RA induced total weight loss was entirely due to greater fat mass loss with no loss in lean mass (FIG. 1C and FIG. 1D).

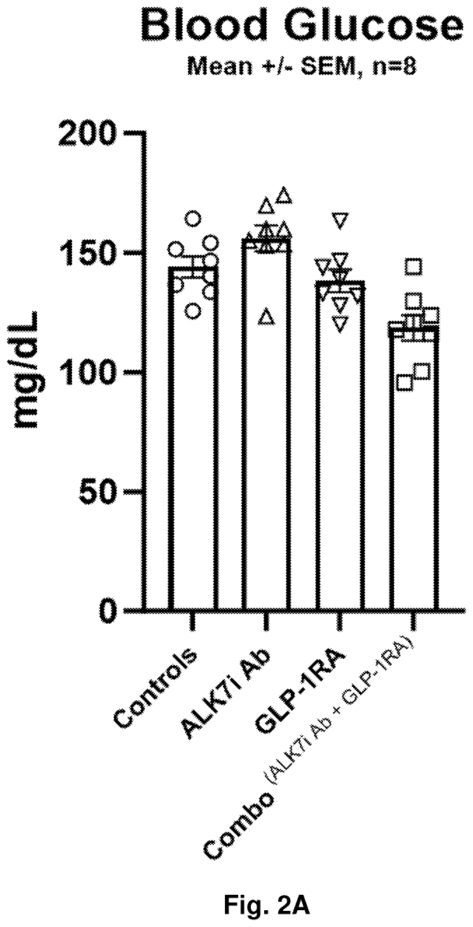

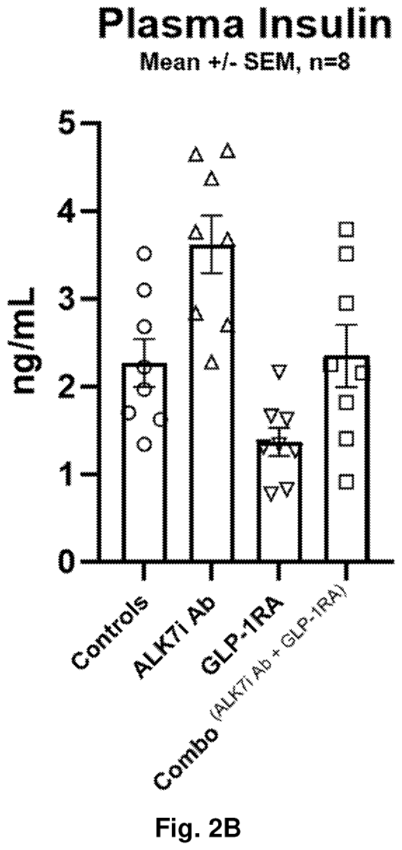

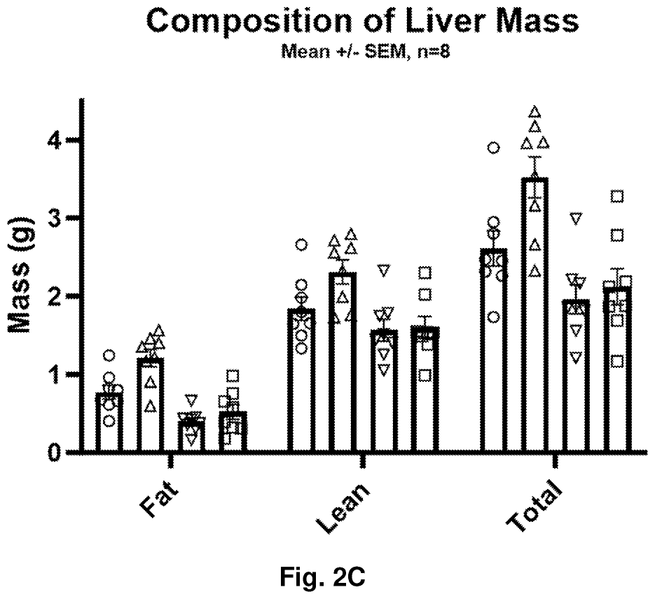

Blood glucose was comparable among control, ALK7i Ab, and GLP-1RA mono treatment groups. Blood glucose tended to be lower in the Combo group (FIG. 2A).

ALK7i Ab mono treatment increased plasma insulin relative to Control group. Plasma insulin was reduced by GLP-1RA mono treatment relative to Control group. Combination treatment of ALK7i Ab and GLP-1RA resulted in comparable fasting insulins relative to Control group (FIG. 2B).

Total Liver mass, liver fat mass, and liver lean mass was increased by ALK7i Ab mono treatment alone relative to Control group. Total Liver mass, liver fat mass, and liver lean mass was reduced by GLP-1RA and Combination ALK7i Ab and GLP-1RA treatment groups relative to Control group (FIG. 2C).

These data indicate that combination therapy of ALK7i Ab and GLP-1RA can result in greater quality of weight loss; greater total fat mass loss, while preserving lean mass, and reducing liver fat mass.

### Example 6: Administration of Anti-ALK7 Receptor Antibody a and GLP-1 Receptor-Amylin Agonist in Mice

The purpose of this Example is to determine the effect of a s.c. injection of an exemplary anti-ALK7 receptor antibody in diet induced obese (DIO) mice on total body weight, fat mass, and lean mass with and without co-administration with a GLP-1 receptor-amylin receptor agonist.

### Study

A four-week study in DIO mice (n=7-8) investigating the body weight lowering effect of human anti-ALK7 inhibiting antibody (ALK7i Ab) alone or in combination with a unimolecular GLP-1 receptor-amylin receptor agonist (GLP1RA-AmyRA). Male, C57BI/6J mice, began high fat diet (HFD) feeding at 10 weeks-old (45% HFD; Research Diets) and throughout the study; study start at 46 weeks of age. Mice were single housed and food intake was measured daily. DIO mice were dosed with an ALK7i Ab (10 mg/kg, s.c., twice weekly), a GLP1RA-AmyRA (1 nmol/kg, s.c. daily), or a combination of the two (Combo group). Control mice were dosed with both vehicle for ALK7i Ab (PBS; s.c. twice weekly) and vehicle for GLP1RA-AmyRA (s.c. daily). Body composition was measured at the end of the study via EchoMRI. On the final study day, mice received final doses of their respective compounds or vehicle at the time fasting began, 4-6 h prior to sacrifice. Immediately prior to sacrifice mice, blood glucose was measured from the tail. Mice were then euthanized and plasma was collected for insulin measurements via ELISA and the liver was dissected out and fat and lean mass content was determined via EchoMRI. The study design was as shown in Table 6.

TABLE 6
Study design.

| | | | Dose | |
|---|---|---|---|---|
| | | | (mg/kg; | Dose |
| | | | twice | (nmol/kg; |
| Group | Tx | Route | weekly) | QD) |
| 1 | Controls | SC | 10 | — |
| 2 | GLP-1-Amylin receptor | SC | — | 1 |
| | agonist of Formula | | | |
| | IV (GLP1RA-AmyRA^{1 nmol/kg}) | | | |
| 3 | Anti-ALK7 antibody A | SC | 10 | — |
| | (ALK7i Ab) | | | |
| 4 | GLP1RA-AmyRA^{3 nmol/kg} of | SC | 10 | 3 |
| | Formula IV | | | |
| 5 | ALK7i Ab A + GLP1RA- | SC | 10 | 1 |
| | AmyRA of Formula IV | | | |
| | (Combo 1) | | | |
| 6 | ALK7i Ab A + GLP1RA- | SC | 10 | 3 |
| | AmyRA of Formula IV | | | |
| | (Combo 2) | | | |

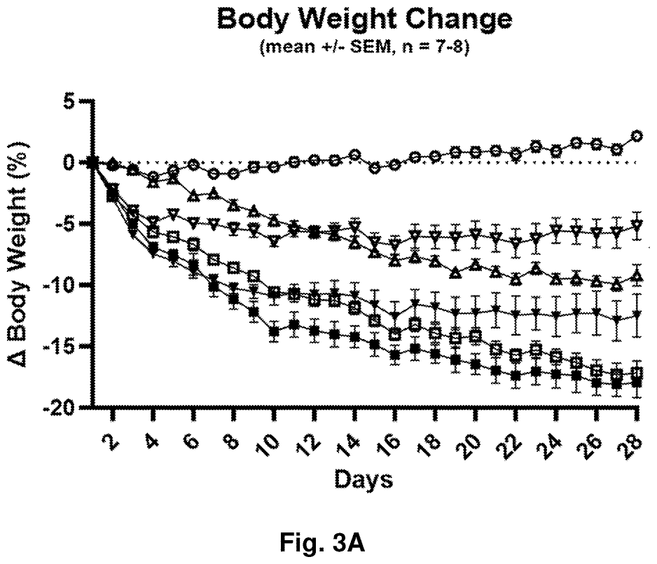

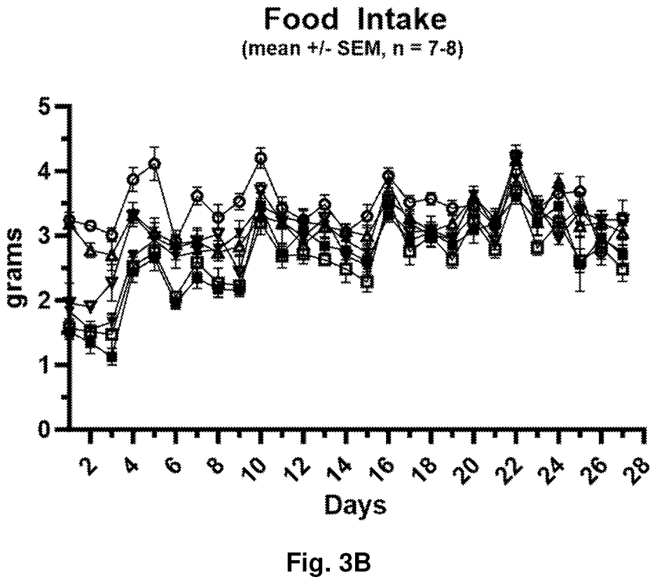

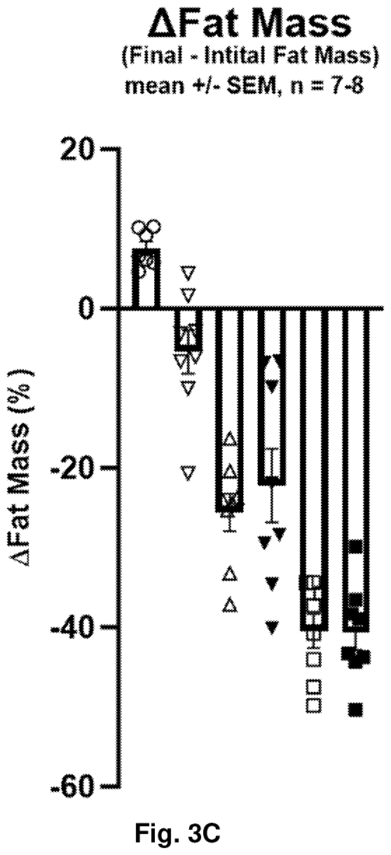

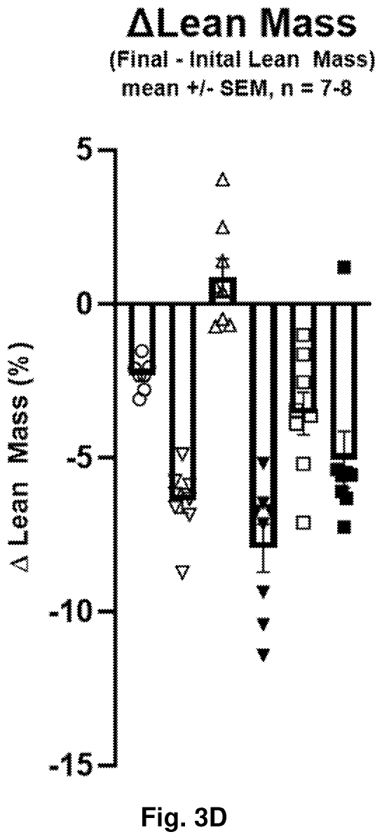

The control group maintained their body weight during the study. All monotherapy treatment groups resulted in body weight loss with: GLP1RA-AmyRA^{1 nmol/kg} approximately 5%, ALK7i Ab lost approximately 9%, and GLP1RA-AmyRA^{3 nmol/kg} approximately 13%. Combination resulted in superior weight loss with Combo 1 losing approximately 17% body weight and Combo 2 group losing approximately 19% body weight (FIG. 3A).

Weight loss with ALK7i Ab mono treatment alone was not associated with reduced food intake. GLP1RA-AmyRA treatment induced weight loss was associated with a transient reduction in food intake and both dose levels, with 3 nmol/kg inducing greater reduction in food intake versus 1 nmol/kg. There was no difference in food intake between GLP1RA-AmyRA^{1 nmol/kg} and Combo 1, despite Combo 1 having greater body weight loss versus GLP1RA-AmyRA^{1 nmol/kg}. Similarly, there was no difference in food intake between GLP1RA-AmyRA^{3 mmol/kg} and Combo 2, despite Combo 2 having greater body weight loss versus GLP1RA-AmyRA^{3 nmol/kg} (FIG. 3B). These data indicate that the synergistic increase in weight loss in the combination groups is due to non-food intake mechanism of action induced by ALK7 inhibition combined with the known food intake reduction effects of GLP1RA and AmyRA.

Mice in the control group gained fat mass and showed a negligible change in lean mass. ALK7i Ab mono treatment induced total weight loss, and this was the result of fat mass loss with no lean mass loss. GLP1RA-AmyRA mono treatment induced total weight loss was due to both fat mass and lean mass loss, with GLP1RA-AmyR^{3 nmol/kg} mono treatment inducing greater loss in both fat mass and lean mass versus GLP1RA-AmyR^{1 nmol/kg} mono treatment. Combo 1 and Combo 2 groups had the greatest total weight loss resulting from greater amount fat mass loss and had blunted lean mass loss relative to their respective GLP1RA-AmyR mono treatment dosed groups. This is consistent with ALK7i preserving lean mass during weight loss (FIG. 3C and FIG. 3D).

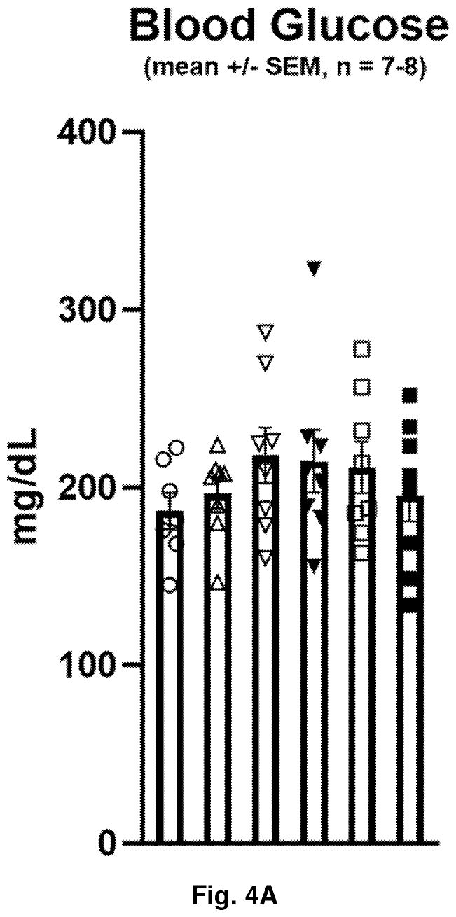

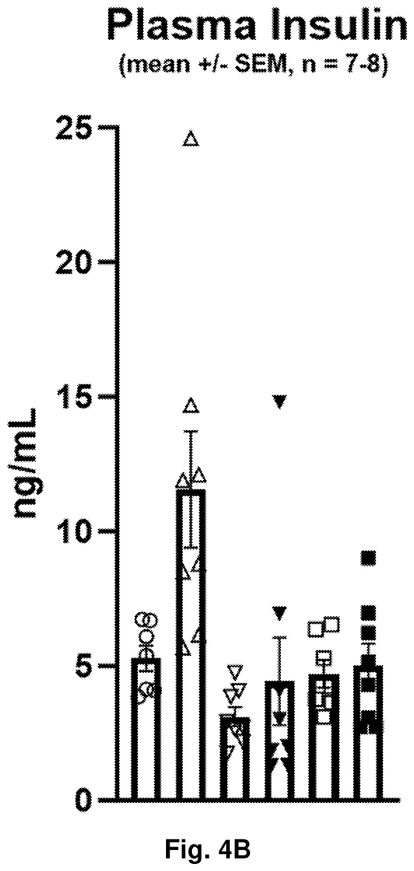

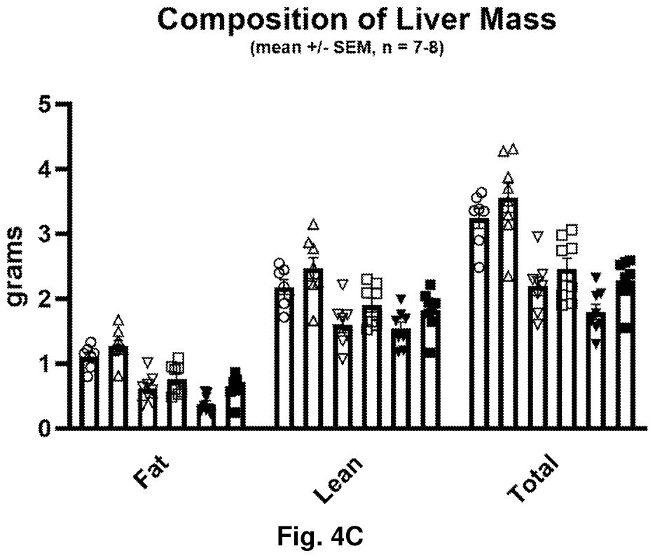

Blood glucose was comparable among control, ALK7i Ab mono treatment, and GLP-1RA-AmyRA (both 1 and 3 nmol/kg) mono treatment groups. Blood glucose tended to be lower in the two Combo groups compared to the two GLP1RA-AmyRA mono treatment groups (FIG. 4A).

ALK7i Ab mono treatment increased plasma insulin relative to the Control group. Plasma insulin was reduced by GLP1RA-AmyRA mono treatments. Combo 1 and 2 eliminated the increased plasma insulin levels resulting from ALK7i Ab mono treatment alone, resulting in comparable fasting insulins relative to the Control group (FIG. 4B).

Total Liver mass, liver fat mass, and liver lean mass was increased by ALK7i Ab mono treatment alone relative to the Control group. Total Liver mass, liver fat mass, and liver lean mass was reduced by GLP1RA-AmyR mono treatments and both Combination ALK7i Ab and GLP1RA-AmyRA treatment groups relative to the Control group (FIG. 4C).

These data indicate that combination therapy of ALK7i Ab and GLP1RA-AmyRA can result in greater quality of weight loss; greater total fat mass loss, while preserving lean mass, and reducing liver fat mass.

### Example 7: Administration of Anti-ALK7 Receptor Antibody a and Anti-GIPR Antibody in Mice

The purpose of this Example is to determine the effect of a s.c. injection of an exemplary anti-ALK7 receptor antibody in diet induced obese (DIO) mice on total body weight, fat mass and lean mass with and without co-administration with an anti-GIP receptor antibody.

### Study

A four-week study in DIO mice (n=8) investigating the body weight lowering effect of human anti-ALK7 receptor inhibiting antibody (ALK7i Ab) in combination with a mouse anti-GIP receptor inhibiting antibody (GIPRi Ab). Male, C57BI/6J mice, began high fat diet (HFD) feeding at 10 weeks-old (45% HFD; Research Diets) and throughout the study; study start at 62 weeks of age. Mice were single housed and food intake was measured daily. Male, DIO mice were dosed with an exemplary ALK7i Ab (10 mg/kg, s.c., twice weekly), an exemplary GIPRi Ab (30 mg/kg, s.c. weekly), or the two in combination (Combo group). Control mice were dosed with both vehicle for ALK7i Ab (PBS; s.c. twice weekly) and vehicle for GIPRi Ab (s.c. weekly). Body composition was measured at the end of the study via EchoMRI. On the final study day, mice received final doses of their respective compounds or vehicle at the time of fast, 4-6 h prior to sacrifice. Immediately prior to sacrifice mice, fasting blood glucose was measured from the tail. Mice were then euthanized and plasma was collected for insulin measurements via ELISA and the liver was dissected out, and liver fat and lean mass content was determined via EchoMRI. The study design was as shown in Table 7.

TABLE 7
Study design.

| | | | Dose: | |
|---|---|---|---|---|
| | | | (mg/kg; | Dose: |
| | | | twice | (mg/kg; |
| Group | Tx | Route | weekly) | QW) |
| 1 | Anti-GLP inhibiting | SC | — | 30 |
| | antibody (GIPRi Ab) | | | |
| 2 | Anti-ALK7 inhibiting | SC | 10 | 30 |
| | antibody A (ALK7i Ab) | | | |
| 3 | ALK7i ab A + GIPR Ab | SC | 10 | 30 |
| | (Combo) | | | |

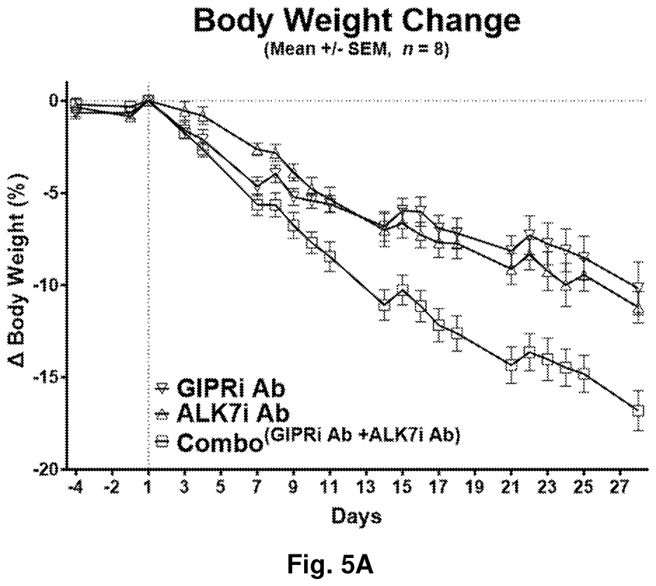

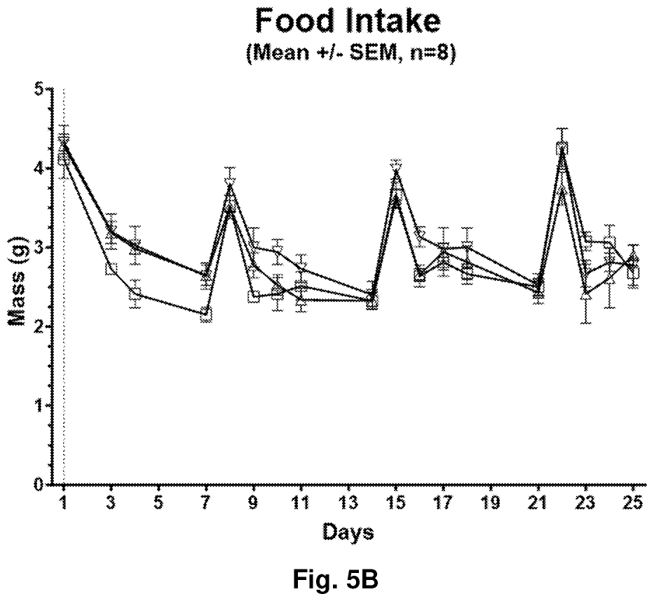

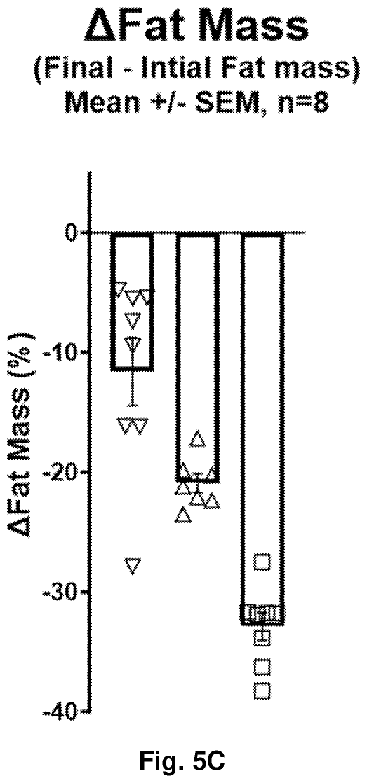

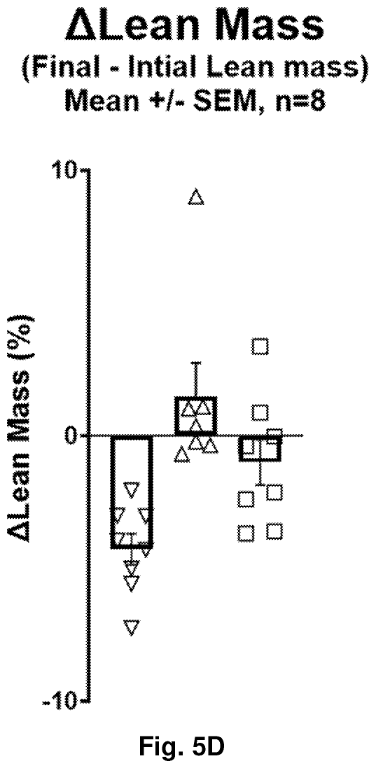

DIO mice treated with GIPRi Ab mono treatment had approximately 10% body weight loss, those treated with ALK7i Ab mono treatment lost approximately 11%, and the Combo group lost approximately 17% body weight (FIG. 5A).

Both GIPRi Ab and ALK7i Ab mono treatment groups had similar food intake. Combo group had modest reduction in food intake relative to mono treatment groups (FIG. 5B).

GIPRi Ab mono treatment induced total weight loss was due to both fat mass and lean mass loss, while ALK7i Ab mono treatment induced total weight loss was only due to fat mass loss and no loss of lean mass. The Combo group had the greatest reduction in total body weight due to increased fat mass loss and prevented loss of lean mass relative to the GIPRi Ab group (FIG. 5C and FIG. 5D).

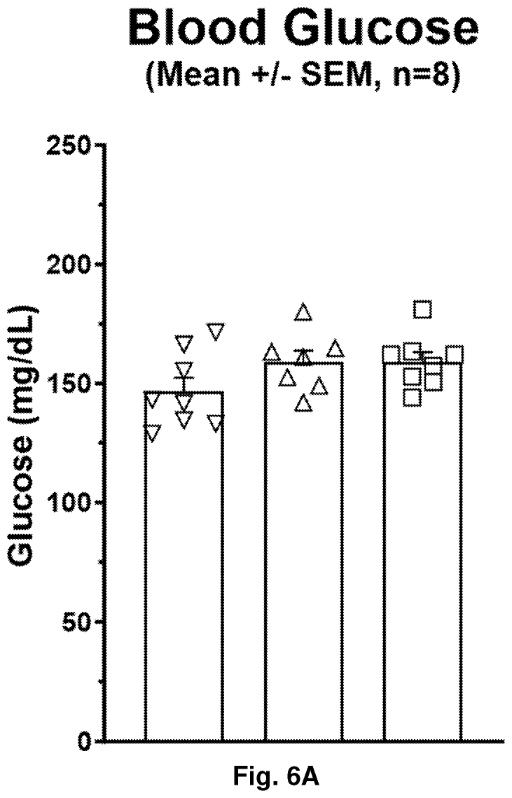

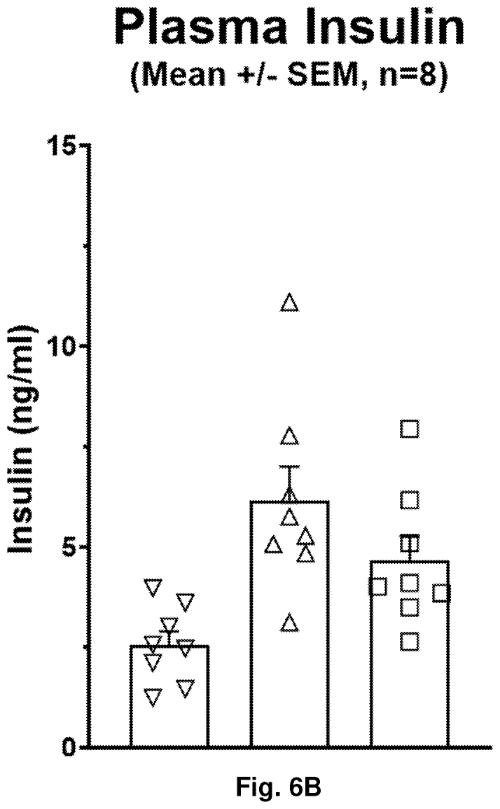

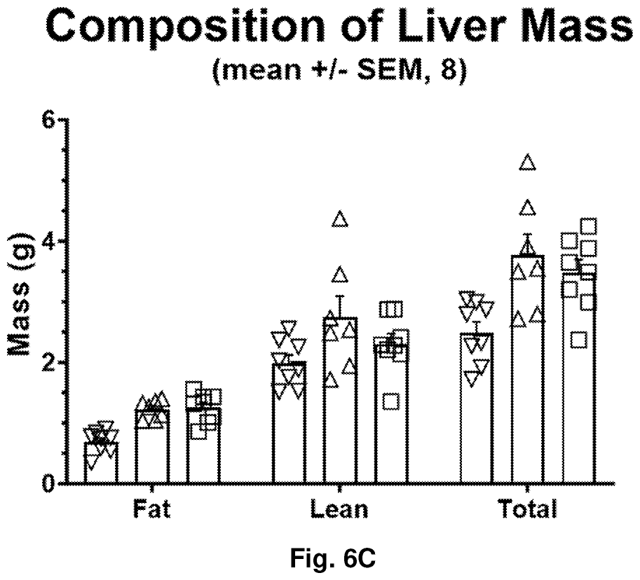

Blood glucose was comparable among GIPRi Ab mono treatment, ALK7i Ab mono treatment, and Combo groups (FIG. 6A).

ALK7i Ab mono treatment increased plasma insulin relative to the GIPRi Ab mono treatment group. Plasma insulin was reduced in Combo relative ALK7i Ab alone, but was elevated relative to the GIPRi Ab group (FIG. 6B).

Total Liver mass, liver fat mass, and liver lean mass was increased by both ALK7i Ab mono treatment and Combo treatment relative to GIPRi Ab mono treatment group (FIG. 6C).

These data indicate that combination therapy of ALK7i Ab and GIPRi Ab can result in greater quality of weight loss; greater total fat mass loss, while preserving lean mass.

### Example 8: Administration of Anti-ALK7 Receptor Antibody a and Anti-Activin Receptor Type II Antibody in Mice

The purpose of this Example is to determine the effect of a s.c. injection of an exemplary anti-ALK7 receptor antibody in diet induced obese (DIO) mice on total body weight, fat mass, and lean mass with and without co-administration with an anti-activin receptor type II antibody. Additionally, the anti-activin receptor type II antibody does not distinguish among various types of activin receptor type IIs. Therefore, a secondary purpose was to determine the contribution of specific anti-ALK7 receptor inhibition (a specific activin receptor subtype 1) versus ubiquitous activin type II receptor inhibition with respect to weight loss and body composition.

### Study

A four-week study in DIO mice (n=6-8) investigating the body weight lowering effect of human anti-ALK7 receptor inhibiting antibody (ALK7i Ab) alone or in combination with a anti-Activin II receptor inhibiting antibody (ActRIIi Ab). Male, C57BI/6J mice, began high fat diet (HFD) feeding at 10 weeks-old (45% HFD; Research Diets) and throughout the study; study start at 63 weeks of age. Mice were single housed and food intake was measured daily. DIO mice were dosed with a vehicle (Controls), an exemplary ALK7i Ab (10 mg/kg, s.c., twice weekly), an exemplary ActRIIi Ab (20 mg/kg, s.c. weekly), or the two in combination (Combo). Control mice were dosed with both vehicle for ALK7i Ab (s.c. twice weekly) and vehicle for ActRlli Ab (s.c. twice weekly). Body composition was measured at the end of the study via EchoMRI. On the final study day, mice received final doses of their respective compounds or vehicle at the time of fast, 4-6 h prior to sacrifice. Immediately prior to sacrifice mice, fasting blood glucose was measured from the tail. Mice were then euthanized and plasma was collected for insulin measurements via ELISA and the liver was dissected out and fat and lean mass content was determined via EchoMRI. The study design was as shown in Table 8.

TABLE 8
Study design.

| | | | Dose: | Dose: |
|---|---|---|---|---|
| | | | (mg/kg; | (mg/kg; |
| | | | twice | twice |
| Group | Tx | Route | weekly) | weekly) |
| 1 | Control | SC | — | — |
| 2 | Anti-ActRII inhibiting | SC | — | 20 |
| | antibody (ActRIIi Ab) | | | |
| 3 | Anti-ALK7 inhibiting | SC | 10 | |
| | antibody A (ALK7i Ab) | | | |
| 4 | ActRi Ab + GIPR Ab | SC | 10 | 20 |
| | (Combo) | | | |

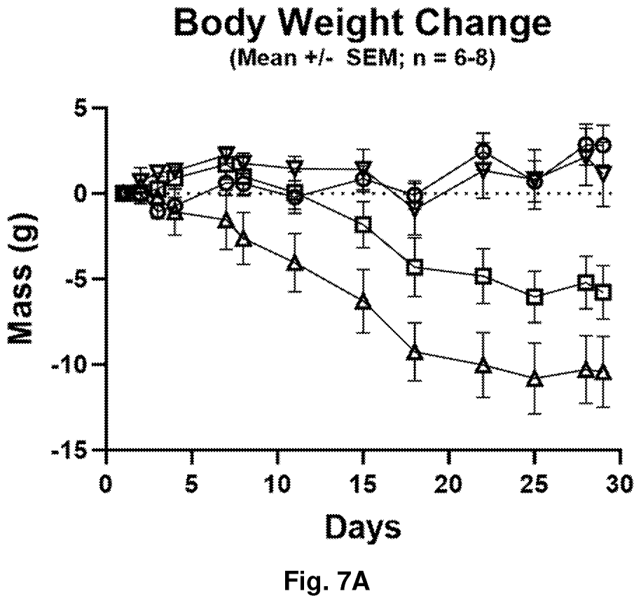

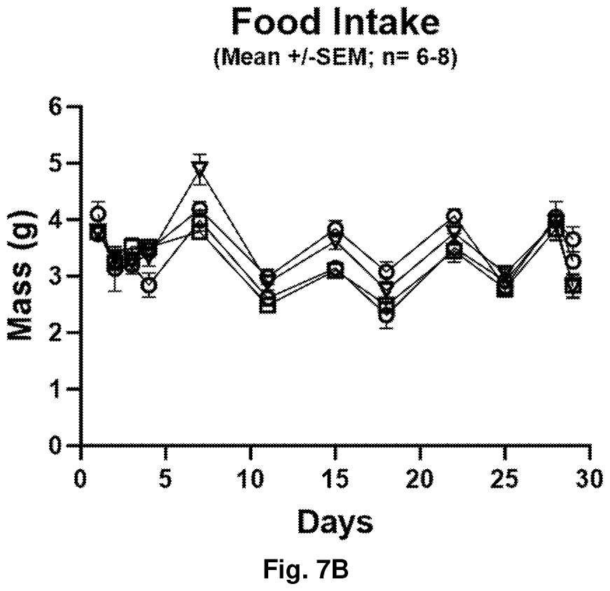

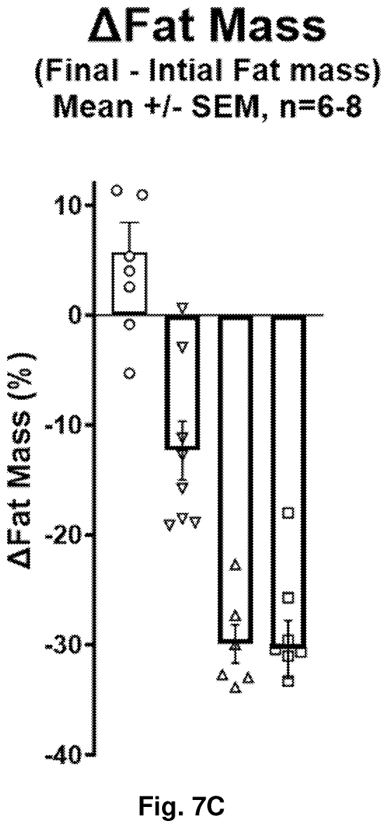

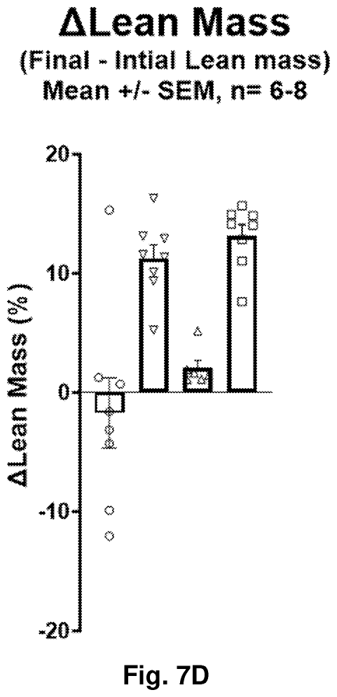

No weight loss was observed in Control or ActRlli groups. DIO mice treated with ALK7i Ab only had approximately 10% body weight loss, and the Combo group lost approximately 6% body weight (FIG. 7A).

Control, ALK7i Ab mono treatment and Combo groups all had similar food intake. However, the ActRIIi Ab had a transient increase in food intake (FIG. 7B). Mice in the control group gained fat mass, and showed an negligible change in lean mass. ActRIIi Ab mono treatment resulted in reduced fat mass with a reciprocal increase in lean mass accounting for the lack of an effect on total body weight. ALK7i Ab mono treatment induced weight loss was due to fat mass loss with no loss of lean mass. The Combo group had similar level of fat mass loss as the ALK7i Ab alone group with a similar increase of lean mass as occurred in the ActRIIi Ab mono treatment group (FIG. 7C and FIG. 7D). Suggesting the anti-activin receptor type II antibody was cable of partially inhibiting ALK7 receptors, as treatment with the specific ALK7 receptor antibody further increased fat mass loss. Concomitantly, the fat mass loss observed in the Combo group was equivalent to the ALK7i Ab group, suggesting the fat mass loss component may be acting thought the ALK7 receptor which was fully inhibited by the ALK7i Ab (i.e., no additive fat mass in Combo group).

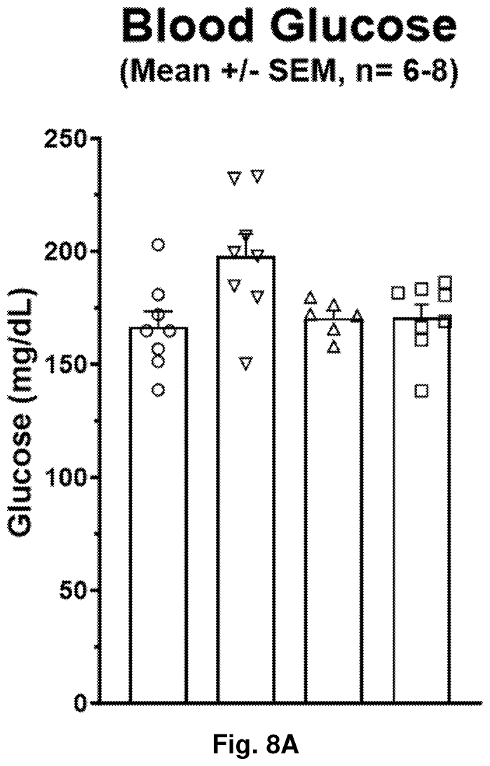

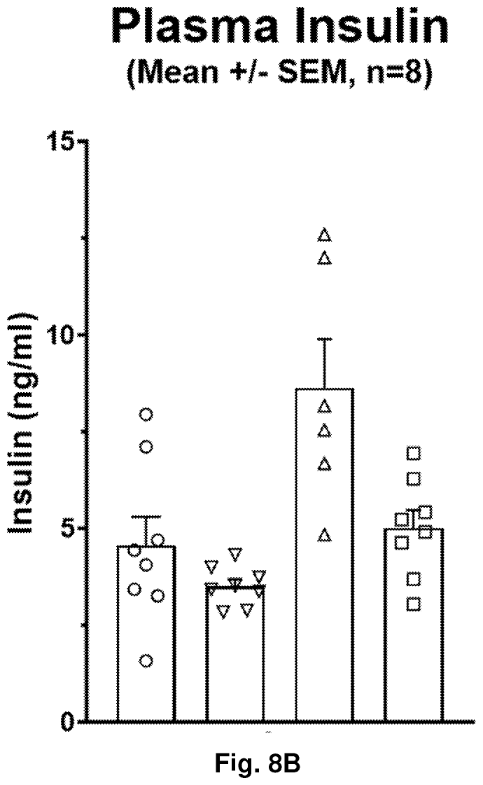

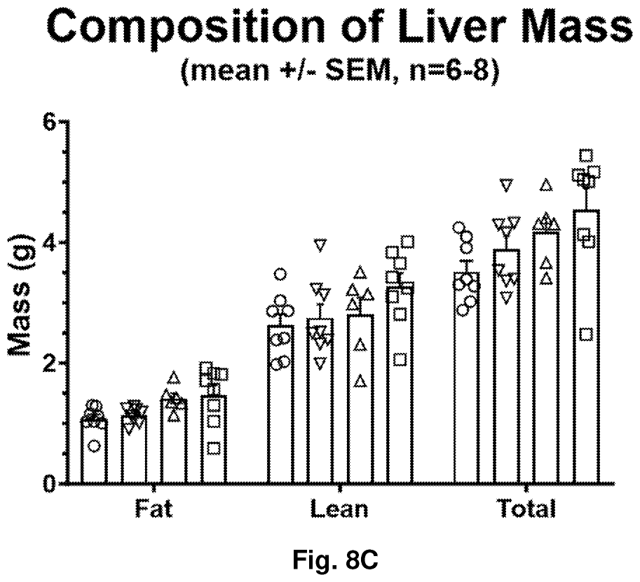

Blood glucose was comparable among Control, ALK7i Ab mono treatment, and Combo groups; however, it was elevated by ActRIIi mono treatment (FIG. 8A).

ALK7i Ab mono treatment increased plasma insulin relative to all other groups. Plasma insulin was reduced in Combo relative ALK7i Ab alone (FIG. 8B).

Total Liver mass, liver fat mass, and liver lean mass was increased by both ALK7i Ab and Combo treatment relative to Control and ActRIIi Ab groups (FIG. 8C).

These data indicate that combination therapy of ALK7i Ab and an anti-activin receptor type II antibody improved body composition; inducing fat mass loss, while increasing lean mass.

### Example 9: Administration of Anti-ALK7 Receptor Antibody B and Anti-Activin Receptor Type II Antibody in Mice

The purpose of this Example is to determine the effect of a s.c. injection of an exemplary anti-ALK7 receptor antibody B in diet induced obese (DIO) mice on total body weight, fat mass, and lean mass with and without co-administration with a GLP-1 RA (of Formula II), or a GLP1RA-AmyRA agonist (of Formula V).

### Study

A four-week study in DIO mice (n=7-8) investigating the body weight lowering effect of human anti-ALK7 inhibiting antibody B (ALK7i Ab) alone or in combination with either a GLP-1 receptor agonist (GLP-1RA) or a unimolecular GLP-1 receptor-amylin receptor agonist (GLP1RA-AmyRA). Male, C57BI/6J mice, began high fat diet (HFD) feeding at 10 weeks-old (45% HFD; Research Diets) and throughout the study; study start at 41 weeks of age. Mice were single housed and food intake was measured daily. DIO mice were dosed with an ALK7i Ab (5 mg/kg, s.c., once weekly), a GLP-1RA (1 nmol/kg, s.c. daily), a GLP1RA-AmyRA (1 nmol/kg, s.c. daily), a combination of ALK7i Ab and GLP-1RA (Combo 1), or a combination of ALK7i Ab and GLP1RA-AmyRA. Control mice were dosed with both vehicle for ALK7i Ab (s.c. once weekly) and vehicle for GLP-1RA or GLP1RA-AmyRA (s.c. daily). Body composition was measured at the end of the study via EchoMRI. Mice received the final dose approximately 24 hours prior to sacrifice. On the final study day, the mice were fasted 4-6 h prior to sacrifice. Immediately prior to sacrifice mice, blood glucose was measured from the tail. Mice were then euthanized and plasma was collected for insulin measurements via ELISA and the liver was dissected out and fat and lean mass content was determined via EchoMRI. The study design was as shown in Table 9.

TABLE 9
Study design.

| | | | Dose: | Dose: |
|---|---|---|---|---|
| | | | (mg/kg; | (nmol/kg; |
| Group | Tx | Route | QW) | QD) |
| 1 | Control | SC | — | — |
| 2 | Anti-ALK7 inhibiting | SC | 5 | — |
| | antibody B (ALK7i Ab) | | | |
| 3 | GLP-1 receptor agonist | SC | — | 1 |
| | B (GLP-1RA B of | | | |
| | Formula II) | | | |
| 4 | ALK7i Ab B + GLP-1RA | SC | 5 | 1 |
| | B of Formula II (Combo 1) | | | |
| 5 | GLP-1-Amylin receptor | SC | — | 1 |
| | agonist B (GLP1RA- | | | |
| | AmyRA of Formula V) | | | |
| 6 | ALK7i Ab B + GLP1RA- | SC | 5 | 1 |
| | AmyRA of Formula V | | | |
| | (Combo 2) | | | |

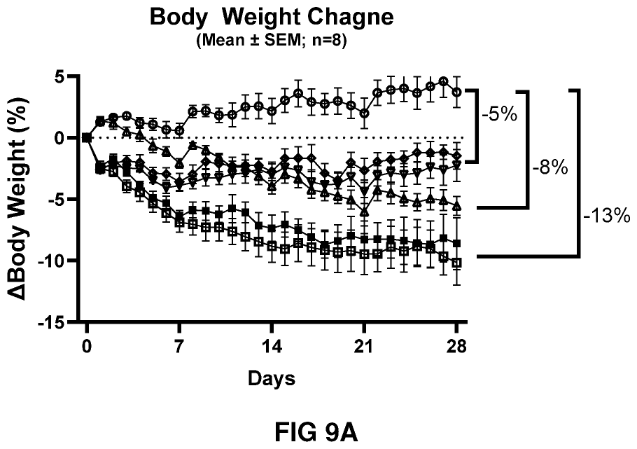

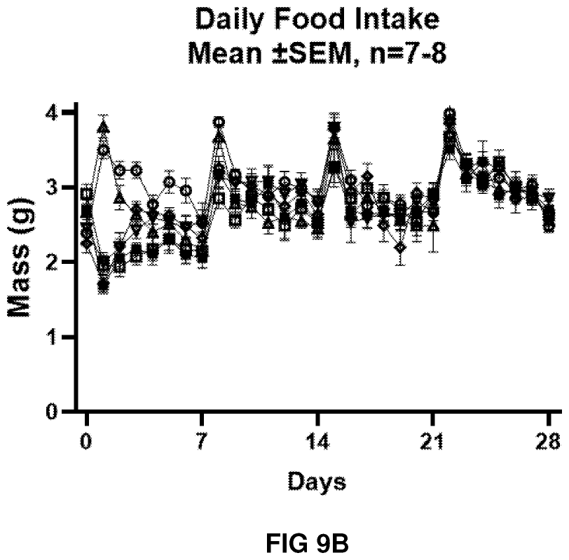

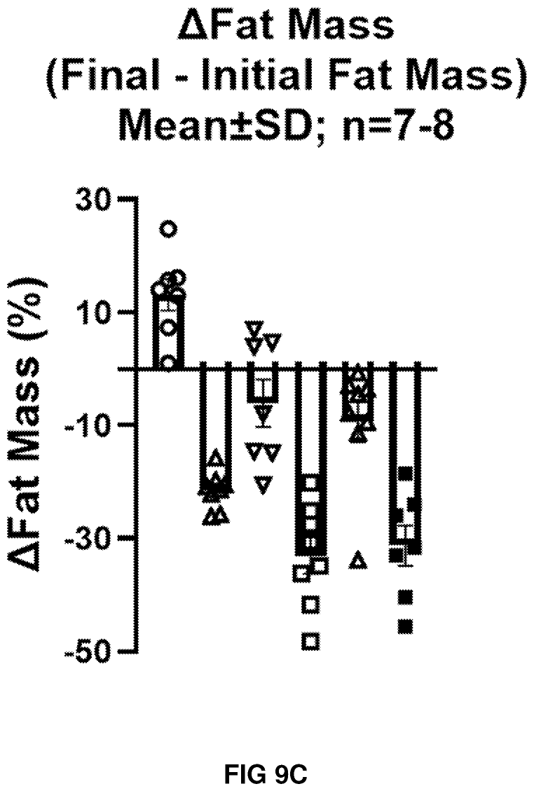

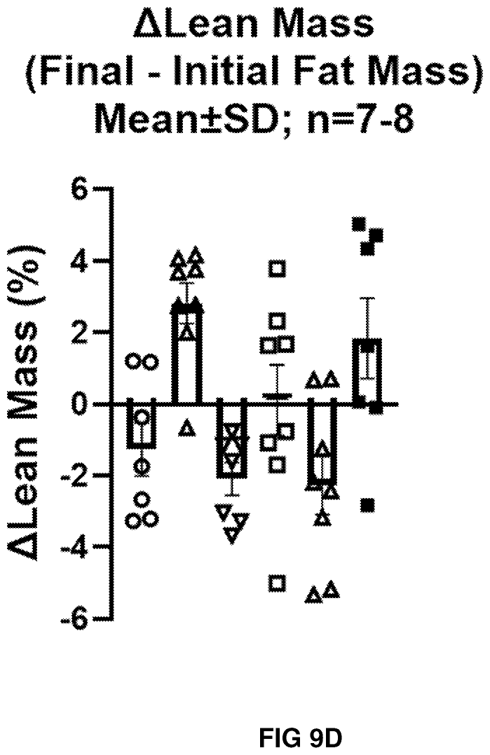

Control mice had a 3% increase in body weight. Mice treated with either GLP-1RA or GLP1RA-AmyRA alone had approximately 5% lower body weight versus control mice. Mice treated with ALK7i Ab only had approximately 8% lower body weight versus control mice, and Combo groups 1 and 2 both had approximately 13% lower body weight versus control mice (FIG. 9A).

Control, ALK7i Ab mono treatment and Combo groups all had similar food intake. All of the groups that received either GLP-1RA or GLP1RA-AmyRA had a transient reduction food intake. However, there was no difference in food intake between GLP-1RA and Combo 1, despite Combo 1 having greater body weight loss versus GLP-1RA. Similarly, there was no difference in food intake between GLP1RA-AmyRA and Combo 2, despite Combo 2 having greater body weight loss versus GLP1RA-AmyRA (FIG. 9B). These data indicate that the synergistic increase in weight loss in the combination groups is due to non-food intake mechanism of action induced by ALK7 inhibition combined with the known food intake reduction effects of GLP1RA and AmyRA.

Mice in the control group gained fat mass and showed a modest reduction in lean mass. ALK7i Ab mono treatment induced total weight loss, and this was the result of fat mass loss with no lean mass loss. GLP-1RA or GLP1RA-AmyR mono treatments both induced total weight loss was due to both fat mass and lean mass loss. Combo 1 and Combo 2 groups had the greatest total weight loss resulting from greater amount fat mass loss and had no lean mass loss. This is consistent with ALK7i preserving lean mass during weight loss (FIG. 9C and FIG. 9D).

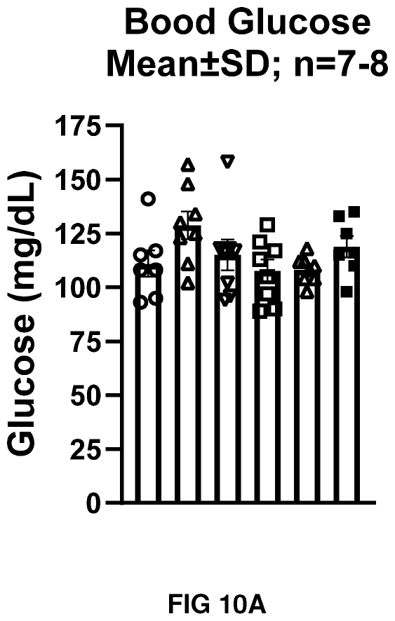

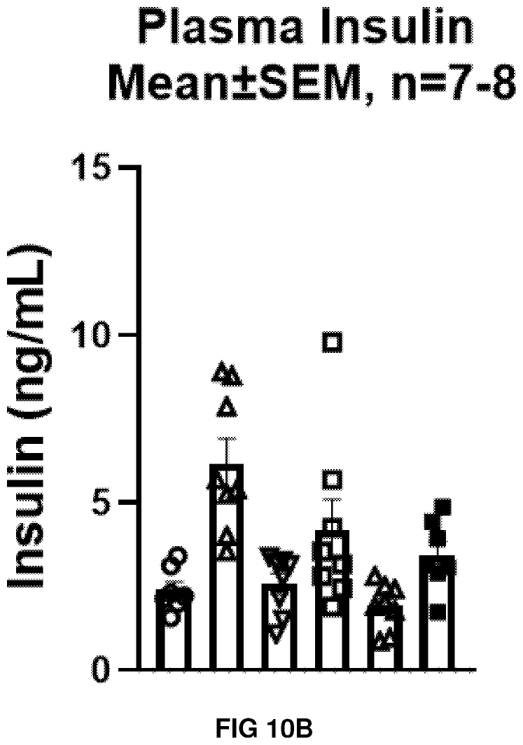

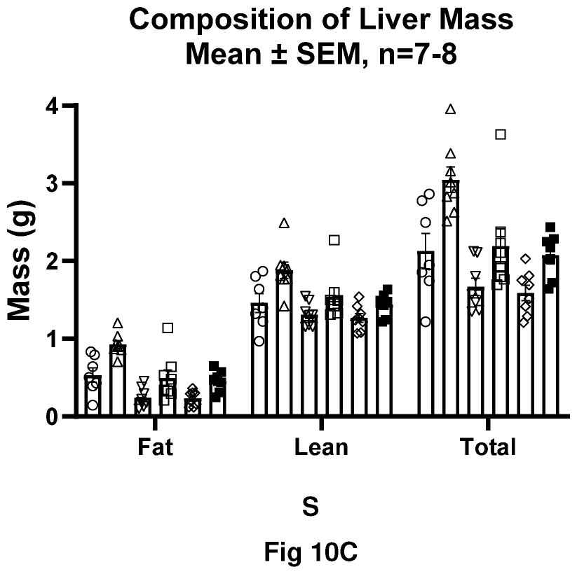

Blood glucose was comparable among control, GLP-1RA and GLP1RA-AmyRA mono treatment groups, Combo 1 and Combo 2 group. ALK7i Ab mono treatment resulted in a modest increase in blood glucose (FIG. 10A).

ALK7i Ab mono treatment increased plasma insulin relative to all groups. Combination treatment of ALK7i Ab with GLP-1RA and GLP1RA-AmyRA both ameliorated the increased insulin levels induced by ALK7iAb alone (FIG. 10B).

Total Liver mass, liver fat mass, and liver lean mass was increased by ALK7i Ab mono treatment alone relative to Control group. Total Liver mass, liver fat mass, and liver lean mass was reduced by GLP-1RA and GLP1RA-AmyRA alone. Combination of ALK7i Ab with either GLP-1RA or GLP1RA-AmyRA ameliorated the increase in ALK7i Ab mono treatment (FIG. 2C).

These data indicate that combination therapy of ALK7i Ab and GLP-1RA or ALK7i Ab and GLP1RA-AmyRA can result in greater quality of weight loss; greater total fat mass loss while preserving lean mass.

While certain features of the invention have been illustrated and described herein, many modifications, substitutions, changes, and equivalents will now occur to those of ordinary skill in the art. It is, therefore, to be understood that the appended embodiments are intended to cover all such modifications and changes as fall within the true spirit of the invention.

1. A method of treating overweight, obesity, and/or diabetes, comprising administering an anti-ALK7 receptor antibody or antigen binding fragment thereof in combination with a second biological active agent, wherein the second biological agent comprises a GLP-1 receptor agonist.

2. The method according to claim 1, wherein the anti-ALK7 receptor antibody or antigen binding fragment thereof is administered:
(i) together with the second biological active agent, or
(ii) concomitantly or sequentially with the second biological active agent.

3. The method of claim 1, wherein the GLP-1 receptor agonist comprises an amino acid sequence having at least 90% sequence identity to the amino acid sequence of SEQ ID NO: 2.

4. The method of claim 1, wherein the GLP-1 receptor agonist comprises the following structure (Formula II): 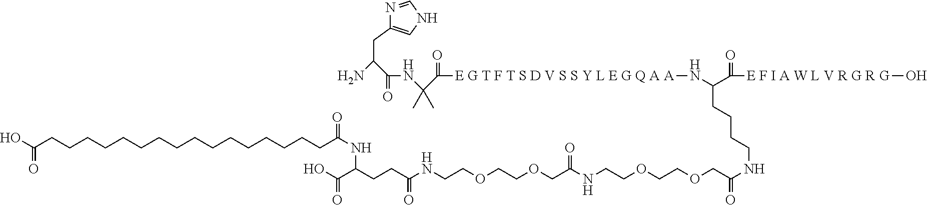
wherein the amino acid sequence is SEQ ID NO: 2, or a pharmaceutically acceptable salt thereof; or
the following structure (Formula I): 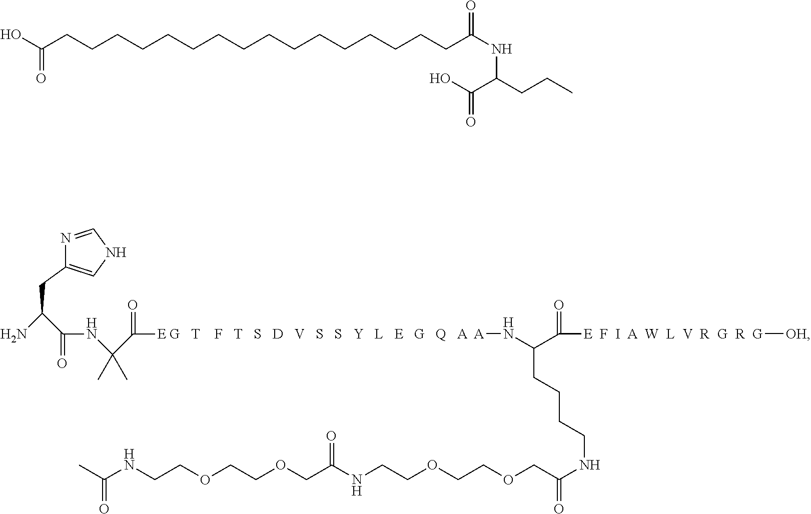
wherein the amino acid sequence is SEQ ID NO: 2, or a pharmaceutically acceptable salt thereof.

5. The method of claim 1, wherein the second biological active agent comprises a GLP-1 receptor agonist and an amylin receptor agonist.

6. The method of claim 1, wherein the second biological active agent comprises a GLP-1 receptor-amylin receptor agonist.

7. The method of claim 6, wherein the GLP-1 receptor-amylin receptor agonist comprises an amino acid sequence having at least 90% sequence identity to the amino acid sequence of SEQ ID NO: 3 or a fragment thereof.

8. The method of claim 6, wherein the GLP-1 receptor-amylin receptor agonist comprises the following structure (formula V): 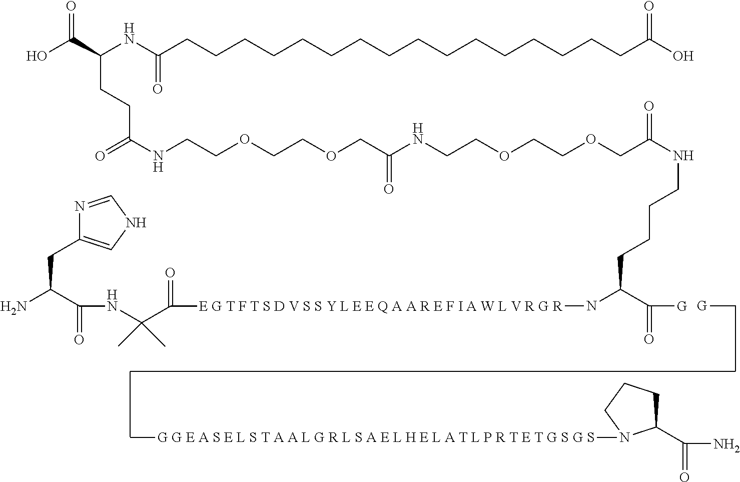
wherein the amino acid sequence is SEQ ID NO: 3, or a pharmaceutically acceptable salt thereof; or comprises the following structure (Formula IV): 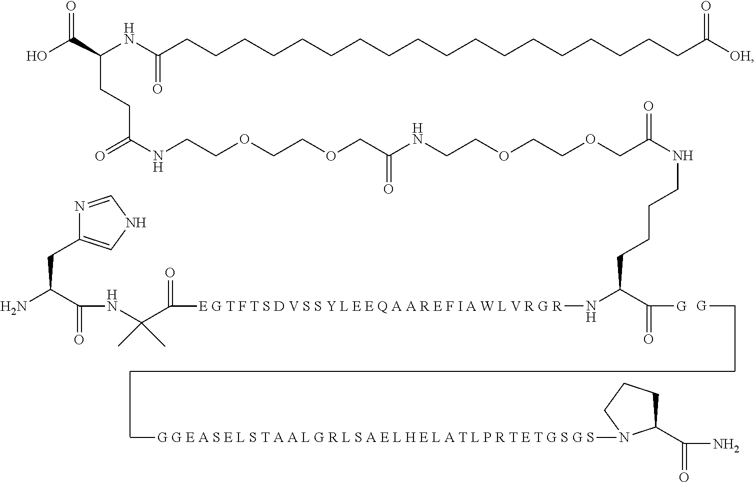
wherein the amino acid sequence is SEQ ID NO: 3, or a pharmaceutically acceptable salt thereof.

9. The method of claim 1, wherein the anti-ALK7 receptor antibody or antigen binding fragment thereof is:
(i) an antagonist;
(ii) characterized in that it blocks binding of activin B to an ALK7 receptor; and/or
(iii) characterized in that it blocks formation of a hexameric signaling complex of an ALK7 receptor with an ActRIIA/B Type II receptor.

10. The method of claim 7, wherein the anti-ALK7 receptor antibody or antigen binding fragment thereof is:
(i) an antagonist;
(ii) characterized in that it blocks binding of activin B to an ALK7 receptor; and/or
(iii) characterized in that it blocks formation of a hexameric signaling complex of an ALK7 receptor with an ActRIIA/B Type II receptor.

11. The method of claim 1, wherein the anti-ALK7 receptor antibody or antigen binding fragment thereof comprises three heavy chain CDR sequences, hCDR1-3, and three light chain CDR, sequences, ICDR1-3, wherein
a) hCDR1 is SGNYWN (SEQ ID NO: 8) or SGSYWN (SEQ ID NO: 9) with up to 1 amino acid substitutions,
b) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid substitutions,
c) hCDR3 is DYYGSEGFGY (SEQ ID NO: 17) or DYYGSEGFAY (SEQ ID NO: 18) with up to 1 or 2 amino acid substitutions,
d) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
e) ICDR2 is WTSTRES (SEQ ID NO: 22) or WASTRES (SEQ ID NO: 23) with up to 1 amino acid substitutions, and
f) ICDR3 is KQSYNLPYT (SEQ ID NO: 24) or KRSYNLPYT (SEQ ID NO: 25) with up to 1 amino acid substitutions.

12. The method of claim 8, wherein the anti-ALK7 receptor antibody or antigen binding fragment thereof comprises three heavy chain CDR sequences, hCDR1-3, and three light chain CDR, sequences, ICDR1-3, wherein
g) hCDR1 is SGNYWN (SEQ ID NO: 8) or SGSYWN (SEQ ID NO: 9) with up to 1 amino acid substitutions,
h) hCDR2 is YISFDGRNNYNPSLKN (SEQ ID NO: 10), YISFDGRTNYNPSLKN (SEQ ID NO: 11), YISFDGRTNYNPSLKS (SEQ ID NO: 12), YISFDGRRNYNPSLKN (SEQ ID NO: 13), YISFTGRTNYNPSLKN (SEQ ID NO: 14), YISFSGRNNYNPSLKN (SEQ ID NO: 15) or YISFSGRTNYNPSLKN (SEQ ID NO: 16) with up to 1 or 2 amino acid substitutions,
i) hCDR3 is DYYGSEGFGY (SEQ ID NO: 17) or DYYGSEGFAY (SEQ ID NO: 18) with up to 1 or 2 amino acid substitutions,
j) ICDR1 is KSSQSLLNSRTRKNYLA (SEQ ID NO: 19), KSSQSLLVSRTRKNYLA (SEQ ID NO: 20) or KSSQSLLYSRTRKNYLA (SEQ ID NO: 21) with up to 1 or 2 amino acid substitutions,
k) ICDR2 is WTSTRES (SEQ ID NO: 22) or WASTRES (SEQ ID NO: 23) with up to 1 amino acid substitutions, and
l) ICDR3 is KQSYNLPYT (SEQ ID NO: 24) or KRSYNLPYT (SEQ ID NO: 25) with up to 1 amino acid substitutions.

13. The method of claim 1, wherein the anti-ALK7 receptor antibody or antigen binding fragment thereof comprises three heavy chain CDR sequences, hCDR1-3, and three light chain CDR, sequences, ICDR1-3, wherein
a) hCDR1 is
(SEQ ID NO: 9)
SGSYWN,
b) hCDR2 is
(SEQ ID NO: 11)
YISFDGRTNYNPSLKN,
c) hCDR3 is
(SEQ ID NO: 18)
DYYGSEGFAY,
d) lCDR1 is
(SEQ ID NO: 20)
KSSQSLLVSRTRKNYLA,
e) lCDR2 is
(SEQ ID NO: 22)
WTSTRES,
and
f) lCDR3 is
(SEQ ID NO: 24)
KQSYNLPYT.

14. The method of claim 7, wherein the anti-ALK7 receptor antibody or antigen binding fragment thereof comprises three heavy chain CDR sequences, hCDR1-3, and three light chain CDR, sequences, ICDR1-3, wherein
g) hCDR1 is
(SEQ ID NO: 9)
SGSYWN,
h) hCDR2 is
(SEQ ID NO: 11)
YISFDGRTNYNPSLKN,
i) hCDR3 is
(SEQ ID NO: 18)
DYYGSEGFAY,
j) lCDR1 is
(SEQ ID NO: 20)
KSSQSLLVSRTRKNYLA,
k) lCDR2 is
(SEQ ID NO: 22)
WTSTRES,
and
lCDR3 is
(SEQ ID NO: 24)
KQSYNLPYT

15. The method of claim 8, wherein the anti-ALK7 receptor antibody or antigen binding fragment thereof comprises three heavy chain CDR sequences, hCDR1-3, and three light chain CDR, sequences, ICDR1-3, wherein
l) hCDR1 is
(SEQ ID NO: 9)
SGSYWN,
m) hCDR2 is
(SEQ ID NO: 11)
YISFDGRTNYNPSLKN,
n) hCDR3 is
(SEQ ID NO: 18)
DYYGSEGFAY,
o) lCDR1 is
(SEQ ID NO: 20)
KSSQSLLVSRTRKNYLA,
p) lCDR2 is
(SEQ ID NO: 22)
WTSTRES,
and
q) lCDR3 is
(SEQ ID NO: 24)
KQSYNLPYT.

16. The method of claim 1, wherein the anti-ALK7 receptor antibody or antigen binding fragment comprises:
i. a heavy chain variable region comprising the amino acid sequence according to SEQ ID NO: 43, and/or
ii. a light chain variable region comprising the amino acid sequence according to SEQ ID NO: 78.

17. The method of claim 8, wherein the anti-ALK7 receptor antibody or antigen binding fragment comprises:
iii. a heavy chain variable region comprising the amino acid sequence according to SEQ ID NO: 43, and/or
iv. a light chain variable region comprising the amino acid sequence according to SEQ ID NO: 78.

18. A method of treating overweight, obesity, and/or diabetes, comprising administering an anti-ALK7 receptor antibody or antigen binding fragment thereof in combination with a second biological active agent, wherein the second biological agent comprises a GLP-1 receptor agonist, wherein the anti-ALK7 receptor antibody or antigen binding fragment comprises a heavy chain comprising the amino acid sequence of SEQ ID NO: 99 and a light chain comprising the amino acid sequence of SEQ ID NO: 100, wherein the second biological is:
(a) GLP-1 receptor agonist comprises the following structure (Formula II): 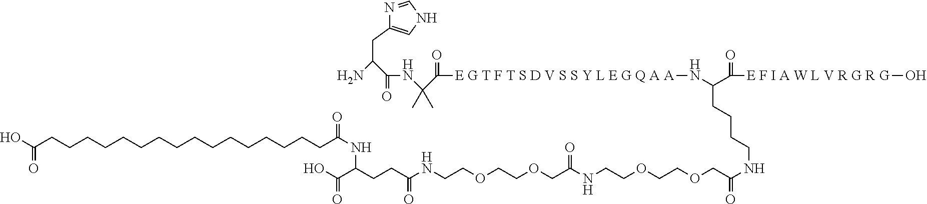
wherein the amino acid sequence is SEQ ID NO: 2, or a pharmaceutically acceptable salt thereof; or
(b) a GLP-1 receptor-amylin receptor agonist comprises the following structure (formula V) 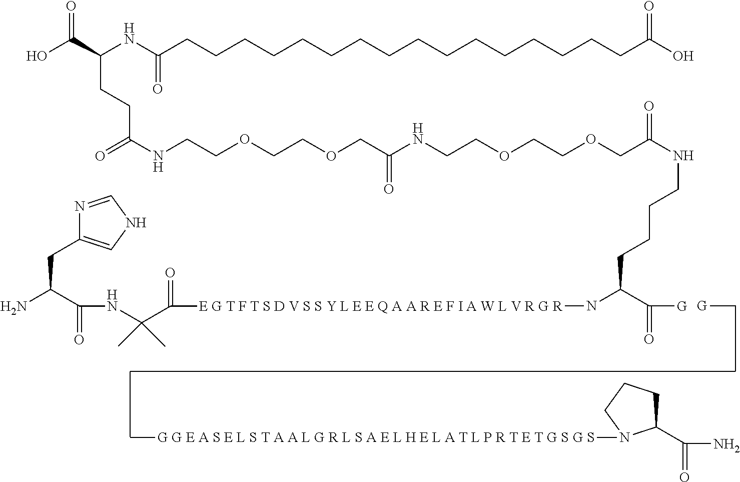
wherein the amino acid sequence is SEQ ID NO: 3, or a pharmaceutically acceptable salt thereof.

19. The method of claim 18, wherein body fat mass is reduced and/or lean body mass is preserved.

20. The method of claim 18, wherein the anti-ALK7 receptor antibody or antigen binding fragment thereof is administered:
(i) together with the second biological active agent, or
(ii) concomitantly or sequentially with the second biological active agent.


SEQUENCE LISTING (109 sequences)

SEQ ID NO: 1 - protein, 493 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 2 - protein, 31 aa, synthetic construct
  SITE 2: note = 2-aminoisobutyric acid
HXEGTFTSDVSSYLEGQAAKEFIAWLVRGRG

SEQ ID NO: 3 - protein, 68 aa, synthetic construct
  SITE 2: note = 2-aminoisobutyric acid
HXEGTFTSDVSSYLEEQAAREFIAWLVRGRKGGGGEASELSTAALGRLSAELHELATLPRTETGSGSP

SEQ ID NO: 4 - protein, 1328 aa, synthetic construct
QSLEESGGDLVRPEASLTLTCTASGFSFYTNYFLCWVRQAPGKGLEWIACIYIGAGGDTYYASWAKGRFTISKTSSTTVTLQMTSLTDADTATYFCTRYVNILDYWFHDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKQSLEESGGDLVRPEASLTLTCTASGFSFYTNYFLCWVRQAPGKGLEWIACIYIGAGGDTYYASWAKGRFTISKTSSTTVTLQMTSLTDADTATYFCTRYVNILDYWFHDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDVVMTQTPASVSESVGGTVTIKCQASQTIGSNLAWYQQKPGQRPKLLIYGASYLASGVPSRFKGSGSGTEYTLTISDLECADAATYYCQCTYTNNNYVGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVVMTQTPASVSESVGGTVTIKCQASQTIGSNLAWYQQKPGQRPKLLIYGASYLASGVPSRFKGSGSGTEYTLTISDLECADAATYYCQCTYTNNNYVGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 1324 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSQSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSQVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 1330 aa, synthetic construct
QVQLVQSGAEVKKPGSSVKVSCKASGGTFSGQAISWVRQAPGQGLEWMGGIIPSFGTARYAQKFQGRVTITADESTSTAYMELSSLRSEDTAVYYCARTPYYDSSGYLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIQMTQSPSSLSASVGDRVTITCQASHDIDNYLNWYQQKPGKAPKLLIYYASNLKTGVPSRFSGSGSGTDFTFTISSLQPEDIATYYCQQSRASPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVKKPGSSVKVSCKASGGTFSGQAISWVRQAPGQGLEWMGGIIPSFGTARYAQKFQGRVTITADESTSTAYMELSSLRSEDTAVYYCARTPYYDSSGYLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIQMTQSPSSLSASVGDRVTITCQASHDIDNYLNWYQQKPGKAPKLLIYYASNLKTGVPSRFSGSGSGTDFTFTISSLQPEDIATYYCQQSRASPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 31 aa, Homo sapiens
HAEGTFTSDVSSYLEGQAAKEFIAWLVKGRG

SEQ ID NO: 8 - protein, 6 aa, synthetic construct
SGNYWN

SEQ ID NO: 9 - protein, 6 aa, synthetic construct
SGSYWN

SEQ ID NO: 10 - protein, 16 aa, synthetic construct
YISFDGRNNYNPSLKN

SEQ ID NO: 11 - protein, 16 aa, synthetic construct
YISFDGRTNYNPSLKN

SEQ ID NO: 12 - protein, 16 aa, synthetic construct
YISFDGRTNYNPSLKS

SEQ ID NO: 13 - protein, 16 aa, synthetic construct
YISFDGRRNYNPSLKN

SEQ ID NO: 14 - protein, 16 aa, synthetic construct
YISFTGRTNYNPSLKN

SEQ ID NO: 15 - protein, 16 aa, synthetic construct
YISFSGRNNYNPSLKN

SEQ ID NO: 16 - protein, 16 aa, synthetic construct
YISFSGRTNYNPSLKN

SEQ ID NO: 17 - protein, 10 aa, synthetic construct
DYYGSEGFGY

SEQ ID NO: 18 - protein, 10 aa, synthetic construct
DYYGSEGFAY

SEQ ID NO: 19 - protein, 17 aa, synthetic construct
KSSQSLLNSRTRKNYLA

SEQ ID NO: 20 - protein, 17 aa, synthetic construct
KSSQSLLVSRTRKNYLA

SEQ ID NO: 21 - protein, 17 aa, synthetic construct
KSSQSLLYSRTRKNYLA

SEQ ID NO: 22 - protein, 7 aa, synthetic construct
WTSTRES

SEQ ID NO: 23 - protein, 7 aa, synthetic construct
WASTRES

SEQ ID NO: 24 - protein, 9 aa, synthetic construct
KQSYNLPYT

SEQ ID NO: 25 - protein, 9 aa, synthetic construct
KRSYNLPYT

SEQ ID NO: 26 - protein, 119 aa, synthetic construct
DVHLQESGPGLVKPSQSLSLTCSVTGHSITSGNYWNWIRQFPGNKLEWMGYISFDGRNNYNPSLKNRISITRDTSKNQFFLRLNSVTTEDTATYFCARDYYGSEGFGYWGQGTLVTVSA

SEQ ID NO: 27 - protein, 119 aa, synthetic construct
DVQVQESGPGPVKPSQSLSLTCSVTGYSITSGSYWNWIRQFPGNKLEWMGYISFDGRNNYNPSLKNRISITRDTSKNQFFLKLNSVTTEDTATYYCARDYYGSEGFAYWGQGTLVTVSA

SEQ ID NO: 28 - protein, 119 aa, synthetic construct
QVHLQESGPGLVKPSQSLSLTCSVSGSSITSGNYWNWIRQFPGNGLEWMGYISFDGRTNYNPSLKNRISITRDTSKNQFSLRLNSVTTEDTATYFCARDYYGSEGFGYWGQGTLVTVSA

SEQ ID NO: 29 - protein, 119 aa, synthetic construct
QVQLQESGPGDVKPSQSLSLTCSVEGYSITSGSYWNWIRQFPGNGLEWIGYISFDGRTNYNPSLKNRISITRDTSKNQFFLKLNSVTTYDTATYYCARDYYGSEGFAYWGQGTLVTVSA

SEQ ID NO: 30 - protein, 119 aa, synthetic construct
QVQVQESGPGPVKPSQSLSLTCSVTGYSITSGSYWNWIRQFPGNGLEWMGYISFDGRTNYNPSLKSRISITRDTSKNQFFLKLNSVTTEDTATYYCARDYYGSEGFAYWGQGTLVTVSA

SEQ ID NO: 31 - protein, 119 aa, synthetic construct
QVQLQESGPGLVKPSQTLSLTCAVPGGSFSSGSYWNWIRQPPGKGLEWIGYISFDGRRNYNPSLKNRVTISVDTSKNQFSLKLSSVTAADTAVYYCARDYYGSEGFAYWGQGTLVTVSS

SEQ ID NO: 32 - protein, 119 aa, synthetic construct
QVQLQESGPGLVKPSQTLSLTCAVSGASITSGNYWNWIRQPPGKGLEWIGYISFDGRTNYNPSLKNRVSISVDTSKNQFSLRLNSVTAADTAVYFCARDYYGSEGFGYWGQGTLVTVSS

SEQ ID NO: 33 - protein, 119 aa, synthetic construct
QVQLQESGPGLVKPSETLSLTCTVSGYSITSGSYWNWIRQPPGKGLEWIGYISFDGRNNYNPSLKNRVTISVDTSKNQFSLKLSSVTAADTAVYYCARDYYGSEGFAYWGQGTLVTVSS

SEQ ID NO: 34 - protein, 119 aa, synthetic construct
EVQLVESGGGLVQPGGSLRLSCAASGYSITSGSYWNWIRQAPGKGLEWMGYISFDGRNNYNPSLKNRITISRDTSKNTFYLQMNSLRAEDTAVYYCARDYYGSEGFAYWGQGTLVTVSS

SEQ ID NO: 35 - protein, 119 aa, synthetic construct
QVQLQESGPGLVKPSQSLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSA

SEQ ID NO: 36 - protein, 119 aa, synthetic construct
QVQLQESGPGLVKPSQSLSLTCSVEGYSITSGSYWNWIRQFPGKGLEWIGYISFDGRTNYNPSLKNRITITRDTSKNQFSLKLNSVTAADTAVYYCARDYYGSEGFGYWGQGTLVTVSA

SEQ ID NO: 37 - protein, 119 aa, synthetic construct
QVQLQESGPGLVKPSQSLSLTCSVSGYSITSGSYWNWIRQPPGKGLEWMGYISFDGRTNYNPSLKSRISISVDTSKNQFFLKLNSVTTEDTAVYYCARDYYGSEGFAYWGQGTLVTVSA

SEQ ID NO: 38 - protein, 119 aa, synthetic construct
QVQLQESGPGLVKPSQSLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 39 - protein, 119 aa, synthetic construct
QVQLQESGPGDVKPSQSLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 40 - protein, 119 aa, synthetic construct
QVQLQESGPGDVKPSQSLSLTCSVEGYSITSGSYWNWIRQPPGKGLEWIGYISFDGRTNYNPSLKNRISISRDTSKNQFSLKLNSVTAADTATYYCARDYYGSEGFAYWGQGTLVTVSS

SEQ ID NO: 41 - protein, 119 aa, synthetic construct
QVQLQESGPGLVKPSQSLSLTCSVSGYSITSGSYWNWIRQPPGKGLEWMGYISFDGRTNYNPSLKSRISISRDTSKNQFFLKLNSVTTEDTAVYYCARDYYGSEGFAYWGQGTLVTVSA

SEQ ID NO: 42 - protein, 119 aa, synthetic construct
QVQLQESGPGLVKPSQSLSLTCSVSGYSITSGSYWNWIRQPPGKGLEWIGYISFDGRTNYNPSLKSRISISRDTSKNQFFLKLSSVTTEDTAVYYCARDYYGSEGFAYWGQGTLVTVSS

SEQ ID NO: 43 - protein, 119 aa, synthetic construct
QVQLQESGPGLVKPSESLSLTCTVEGYSITSGSYWNWIRQPPGKGLEWIGYISFDGRTNYNPSLKNRVTISRDTSKNQFSLKLSSVTAADTAVYYCARDYYGSEGFAYWGQGTLVTVSS

SEQ ID NO: 44 - protein, 119 aa, synthetic construct
QVQLQESGPGLVKPSESLSLTCTVEGYSITSGSYWNWIRQPPGKGLEWIGYISFDGRTNYNPSLKNRVTISVDTSKNQFSLKLSSVTAADTATYYCARDYYGSEGFAYWGQGTLVTVSS

SEQ ID NO: 45 - protein, 119 aa, synthetic construct
QVQLQESGPGLVKPSETLSLTCTVSGYSITSGSYWNWIRQPPGKGLEWIGYISFSGRNNYNPSLKNRVTISVDTSKNQFSLKLSSVTAADTAVYYCARDYYGSEGFAYWGQGTLVTVSS

SEQ ID NO: 46 - protein, 119 aa, synthetic construct
QVQLQESGPGLVKPSQSLSLTCSVEGYSITSGSYWNWIRQPPGKGLEWIGYISFDGRTNYNPSLKNRVTISRDTSKNQFSLKLNSVTAADTAVYYCARDYYGSEGFAYWGQGTLVTVSS

SEQ ID NO: 47 - protein, 119 aa, synthetic construct
QVQLQESGPGLVKPSQTLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 48 - protein, 119 aa, synthetic construct
QVQLQESGPGLVKPSQSLSLTCTVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 49 - protein, 119 aa, synthetic construct
QVQLQESGPGLVKPSQSLSLTCSVEGYSITSGSYWNWIRQPPGKGLEWIGYISFDGRTNYNPSLKNRVTISRDTSKNQFSLKLSSVTAADTAVYYCARDYYGSEGFAYWGQGTLVTVSS

SEQ ID NO: 50 - protein, 119 aa, synthetic construct
QVQLQESGPGLVKPSESLSLTCTVEGYSITSGSYWNWIRQPPGKGLEWIGYISFDGRTNYNPSLKNRVTISRDTSKNQFSLKLNSVTAADTAVYYCARDYYGSEGFAYWGQGTLVTVSS

SEQ ID NO: 51 - protein, 119 aa, synthetic construct
QVQLQESGPGLVKPSETLSLTCTVEGYSITSGSYWNWIRQPPGKGLEWIGYISFDGRTNYNPSLKNRVTISRDTSKNQFSLKLSSVTAADTATYYCARDYYGSEGFAYWGQGTLVTVSS

SEQ ID NO: 52 - protein, 119 aa, synthetic construct
QVQLQESGPGLVKPSQSLSLTCSVEGYSITSGSYWNWIRQPPGKGLEWIGYISFDGRTNYNPSLKNRVTISRDTSKNQFSLKLSSVTAADTATYYCARDYYGSEGFAYWGQGTLVTVSS

SEQ ID NO: 53 - protein, 119 aa, synthetic construct
QVQLQESGPGLVKPSETLSLTCSVEGYSITSGSYWNWIRQPPGKGLEWIGYISFTGRTNYNPSLKNRVTISRDTSKNQFSLKLNSVTAADTATYYCARDYYGSEGFAYWGQGTLVTVSS

SEQ ID NO: 54 - protein, 119 aa, synthetic construct
QVQLQESGPGLVKPSQSLSLTCSVEGYSITSGSYWNWIRQPPGKGLEWIGYISFSGRTNYNPSLKNRVTISRDTSKNQFSLKLNSVTAADTATYYCARDYYGSEGFAYWGQGTLVTVSS

SEQ ID NO: 55 - protein, 119 aa, synthetic construct
QVQLQESGPGLVKPSETLSLTCSVEGYSITSGSYWNWIRQPPGKGLEWIGYISFSGRTNYNPSLKNRVTISRDTSKNQFSLKLNSVTAADTATYYCARDYYGSEGFAYWGQGTLVTVSS

SEQ ID NO: 56 - protein, 119 aa, synthetic construct
QVQLQESGPGLVKPSESLSLTCTVEGYSITSGSYWNWIRQPPGKGLEWIGYISFDGRTNYNPSLKNRVTISVDTSKNQFSLKLSSVTAADTAVYYCARDYYGSEGFAYWGQGTLVTVSS

SEQ ID NO: 57 - protein, 119 aa, synthetic construct
QVQLQESGPGLVKPSESLSLTCTVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 58 - protein, 119 aa, synthetic construct
QVQLQESGPGLVKPSESLSLTCTVEGYSITSGSYWNWIRQPPGKGLEWIGYISFDGRTNYNPSLKNRVTISRDTSKNQFSLKLSSVTAADTATYYCARDYYGSEGFAYWGQGTLVTVSS

SEQ ID NO: 59 - protein, 119 aa, synthetic construct
QVQLQESGPGLVKPSQTLSLTCTVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 60 - protein, 113 aa, synthetic construct
DIVMSQSPSSLAVSAGEKVTMSCKSSQSLLNSRTRKNYLAWFQQKPGQSPKLLIYWTSTRESGVPDRFTGSGSGTDFTLTISSVQAEDLAVYYCKQSYNLPYTFGGGTKLEIK

SEQ ID NO: 61 - protein, 113 aa, synthetic construct
DNVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLPYTFGGGTKLEIK

SEQ ID NO: 62 - protein, 113 aa, synthetic construct
DIVMSQSPSSLAVSAGEKVTMSCKSSQSLLNSRTRKNYLAWFQQKPGQSPKLLIYWTSTRESGVPDRFTGSGSGTDFTLTISSVQAEDLAVYYCKRSYNLPYTFGGGTKLEIK

SEQ ID NO: 63 - protein, 113 aa, synthetic construct
DIVMTQSPMSLAVSLGERATINCKSSQSLLVSRTRKNYLAWYQQKPGQPPKLLIYWASTRESGVPDRFSGSGSGTDFTLTISSLQAEDVAVYYCKQSYNLPYTFGQGTKLEIK

SEQ ID NO: 64 - protein, 113 aa, synthetic construct
DIVMTQSPISLAVSLGERATINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 65 - protein, 113 aa, synthetic construct
DIVMTQSPDSLAVSAGEHVTINCKSSQSLLVSRTRKNYLAWYQQKPGQPPKLLIYWASTRESGVPDRFSGSGSGTDFTLTISSVQAEDVAVYYCKQSYNLPYTFGQGTKLEIK

SEQ ID NO: 66 - protein, 113 aa, synthetic construct
DIVMSQSPSSLAVSLGEDVTMHCKSSQSLLNSRTRKNYLAWYQQKPGQSPKLLIYWASTRESGVPDRFTGSGSGTDFTLTISSVQAEDLAVYYCKQSYNLPYTFGEGTKLEIK

SEQ ID NO: 67 - protein, 113 aa, synthetic construct
DIVMTQSPYSLAVSLGERATINCKSSQSLLYSRTRKNYLAWYQQKPGQPPKLLIYWASTRESGVPDRFSGSGSGTDFTLTISSLQAEDVAVYYCKQSYNLPYTFGQGTKLEIK

SEQ ID NO: 68 - protein, 113 aa, synthetic construct
DIVMTQSPDSLAVSLGERATINCKSSQSLLNSRTRKNYLAWFQQKPGQSPKLLIYWTSTRESGVPDRFSGSGSGTDFTLTISSLQAEDVAVYYCKQSYNLPYTFGGGTKVEIK

SEQ ID NO: 69 - protein, 113 aa, synthetic construct
DIVMTQSPDSLAVSLGERATINCKSSQSLLNSRTRKNYLAWYQQKPGQPPKLLIYWTSTRESGVPDRFSGSGSGTDFTLTISSLQAEDVAVYYCKQSYNLPYTFGGGTKVEIK

SEQ ID NO: 70 - protein, 113 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLPYTFGGGTKVEIK

SEQ ID NO: 71 - protein, 113 aa, synthetic construct
DIVMSQSPDSLAVSLGEDVTMHCKSSQSLLNSRTRKNYLAWYQQKPGQSPKLLIYWASTRESGVPDRFTGSGSGTDFTLTISSVQAEDVAVYYCKQSYNLPYTFGQGTKLEIK

SEQ ID NO: 72 - protein, 113 aa, synthetic construct
DIVMTQSPISLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 73 - protein, 113 aa, synthetic construct
DIVMTQSPISLAVSLGERATINCKSSQSLLYSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 74 - protein, 113 aa, synthetic construct
DIVMTQSPDSLAVSLGERVTINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 75 - protein, 113 aa, synthetic construct
DIVMTQSPDSLAVSLGERATINCKSSQSLLYSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 76 - protein, 113 aa, synthetic construct
DIVMTQSPDSLAVSLGEDVTMHCKSSQSLLYSRTRKNYLAWYQQKPGQPPKLLIYWASTRESGVPDRFTGSGSGTDFTLTISSVQAEDVAVYYCKQSYNLPYTFGQGTKLEIK

SEQ ID NO: 77 - protein, 113 aa, synthetic construct
DIVMTQSPDSLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 78 - protein, 113 aa, synthetic construct
DIVMTQSPDSLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGGGTKLEIK

SEQ ID NO: 79 - protein, 113 aa, synthetic construct
DIVMTQSPDSLAVSLGERATINCKSSQSLLVSRTRKNYLAWFQQKPGQSPKLLIYWTSTRESGVPDRFSGSGSGTDFTLTISSLQAEDVAVYYCKQSYNLPYTFGGGTKVEIK

SEQ ID NO: 80 - protein, 113 aa, synthetic construct
DIVMTQSPDSLAVSLGEDATINCKSSQSLLYSRTRKNYLAWFQQKPGQSPKLLIYWTSTRESGVPDRFSGSGSGTDFTLTISSLQAEDVAVYYCKQSYNLPYTFGGGTKVEIK

SEQ ID NO: 81 - protein, 113 aa, synthetic construct
DIVMTQSPDSLAVSLGEDATINCKSSQSLLYSRTRKNYLAWFQQKPGQPPKLLIYWTSTRESGVPDRFSGSGSGTDFTLTISSLQAEDVAVYYCKQSYNLPYTFGGGTKVEIK

SEQ ID NO: 82 - protein, 113 aa, synthetic construct
DIVMTQSPDSLAVSLGEDATINCKSSQSLLYSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 83 - protein, 113 aa, synthetic construct
DIVMTQSPDSLAVSLGEDATINCKSSQSLLYSRTRKNYLAWYQQKPGQPPKLLIYWTSTRESGVPDRFSGSGSGTDFTLTISSLQAEDVAVYYCKQSYNLPYTFGGGTKLEIK

SEQ ID NO: 84 - protein, 113 aa, synthetic construct
DIVMTQSPISLAVSLGEDATINCKSSQSLLYSRTRKNYLAWYQQKPGQPPKLLIYWTSTRESGVPDRFSGSGSGTDFTLTISSLQAEDVAVYYCKQSYNLPYTFGGGTKLEIK

SEQ ID NO: 85 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 107 aa, synthetic construct [sequence omitted]

SEQ ID NO: 87 - protein, 108 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 88 - protein, 107 aa, synthetic construct [sequence omitted]

SEQ ID NO: 89 - protein, 713 aa, synthetic construct
LKCVCLLCDSSNFTCQTEGACWASVMLTNGKEQVIKSCVSLPELNAQVFCHSSNNVTKTECCFTDFCNNITLHLPTASPNAPKLGPMEGGGGQGGGGQGGGGQDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGQGGGGQGGGGQGGHHHHHHHH

SEQ ID NO: 90 - protein, 331 aa, synthetic construct
LKCVCLLCDSSNFTCQTEGACWASVMLTNGKEQVIKSCVSLPELNAQVFCHSSNNVTKTECCFTDFCNNITLHLPTASPNAPKLGPMEGGGGAEPGAEPGAEPGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 31 aa, synthetic construct
  SITE 2: note = 2-aminoisobutyric acid
HXEGTFTSDVSSYLEEQAAREFIAWLVRGRK

SEQ ID NO: 92 - protein, 32 aa, synthetic construct
ASELSTAALGRLSAELHELATLPRTETGSGSP

SEQ ID NO: 93 - protein, 448 aa, synthetic construct
QSLEESGGDLVRPEASLTLTCTASGFSFYTNYFLCWVRQAPGKGLEWIACIYIGAGGDTYYASWAKGRFTISKTSSTTVTLQMTSLTDADTATYFCTRYVNILDYWFHDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 216 aa, synthetic construct
DVVMTQTPASVSESVGGTVTIKCQASQTIGSNLAWYQQKPGQRPKLLIYGASYLASGVPSRFKGSGSGTEYTLTISDLECADAATYYCQCTYTNNNYVGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 445 aa, synthetic construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 217 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - protein, 451 aa, synthetic construct
QVQLVQSGAEVKKPGSSVKVSCKASGGTFSGQAISWVRQAPGQGLEWMGGIIPSFGTARYAQKFQGRVTITADESTSTAYMELSSLRSEDTAVYYCARTPYYDSSGYLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 214 aa, synthetic construct
DIQMTQSPSSLSASVGDRVTITCQASHDIDNYLNWYQQKPGKAPKLLIYYASNLKTGVPSRFSGSGSGTDFTFTISSLQPEDIATYYCQQSRASPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 449 aa, synthetic construct
QVQLQESGPGLVKPSESLSLTCTVEGYSITSGSYWNWIRQPPGKGLEWIGYISFDGRTNYNPSLKNRVTISRDTSKNQFSLKLSSVTAADTAVYYCARDYYGSEGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 220 aa, synthetic construct
DIVMTQSPDSLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 6 aa, synthetic construct
  VARIANT 1..6: note = This sequence may encompass 1-6 residues
GGGGGG

SEQ ID NO: 102 - protein, 4 aa, synthetic construct
GGGG

SEQ ID NO: 103 - protein, 5 aa, synthetic construct
GGGGE

SEQ ID NO: 104 - protein, 68 aa, synthetic construct; identical to SEQ ID NO: 3
  SITE 2: note = 2-aminoisobutyric acid
  SITE 31: note = Lysine modified with [2-[2-[2-[[2-[2-[2-[[(4S)-4-ca rboxy-4-(17-carboxyheptadecanoylamino)butanoyl]amino]ethox y]ethoxy]acetyl]amino] ethoxy]ethoxy]acetyl]
  SITE 68: note = Amidated residue

SEQ ID NO: 105 - protein, 68 aa, synthetic construct; identical to SEQ ID NO: 3
  SITE 2: note = 2-aminoisobutyric acid
  SITE 31: note = Lysine modified with [2-[2-[2-[[2-[2-[2-[[(4S)-4-ca rboxy-4-(19-carboxynonadecanoylamino)butanoyl]amino]ethoxy ]ethoxy]acetyl]amino]ethoxy]ethoxy]acetyl]
  SITE 68: note = Amidated residue

SEQ ID NO: 106 - protein, 31 aa, synthetic construct; identical to SEQ ID NO: 2
  SITE 2: note = 2-aminoisobutyric acid
  SITE 20: note = Lysine modified with [2-[2-[2-[[2-[2-[2-[[(4R)-4-ca rboxy-4-(17-carboxyheptadecanoylamino)butanoyl]amino]ethox y]ethoxy]acetyl]amino]ethoxy]ethoxy]acetyl]

SEQ ID NO: 107 - protein, 31 aa, synthetic construct; identical to SEQ ID NO: 2
  SITE 2: note = 2-aminoisobutyric acid
  SITE 20: note = Lysine modified with [2-[2-[2-[[2-[2-[2-[[(4S)-4-ca rboxy-4-(17-carboxyheptadecanoylamino)butanoyl]amino]ethox y]ethoxy]acetyl]amino]ethoxy]ethoxy]acetyl]

SEQ ID NO: 108 - protein, 68 aa, synthetic construct; identical to SEQ ID NO: 3
  SITE 2: note = 2-aminoisobutyric acid
  SITE 31: note = Lysine modified with [2-[2-[2-[[2-[2-[2-[[(4S)-4-ca rboxy-4-(19-carboxynonadecanoylamino)butanoyl]amino]ethoxy ]ethoxy]acetyl]amino]ethoxy]ethoxy]acetyl]
  SITE 68: note = Amidated residue

SEQ ID NO: 109 - protein, 68 aa, synthetic construct; identical to SEQ ID NO: 3
  SITE 2: note = 2-aminoisobutyric acid
  SITE 31: note = Lysine modified with [2-[2-[2-[[2-[2-[2-[[(4S)-4-ca rboxy-4-(17-carboxyheptadecanoylamino)butanoyl]amino]ethox y]ethoxy]acetyl]amino]ethoxy]ethoxy]acetyl]
  SITE 68: note = Amidated residue